(19) United States
(12) Patent Application Publication
(10) Pub. No.: US 2012/0100549 A1
(43) Pub. Date: Apr. 26, 2012

(54) TARGETED GENOME AMPLIFICATION METHODS

(75) Inventors: Mark Eshoo, Solana Beach, CA (US); Christopher Crowder, San Marcos, CA (US); John Picuri, Carlsbad, CA (US); Neill White, Temecula, CA (US); Megan A. Rounds, Carlsbad, CA (US)

(73) Assignee: IBIS BIOSCIENCES, INC., Carlsbad, CA (US)

(21) Appl. No.: 13/250,173

(22) Filed: Sep. 30, 2011

Related U.S. Application Data
(60) Provisional application No. 61/388,985, filed on Oct. 1, 2010.
(60) Provisional application No. 61/428,652, filed on Dec. 30, 2010.

(51) Int. Cl. C12Q 1/68

(52) U.S. Cl. 435/6.12

(57) ABSTRACT

The methods disclosed herein relate to methods and compositions for amplifying nucleic acid sequences, more specifically, from nucleic acid sequences of pathogens by targeted genome amplification. In certain embodiments, multiple primer pairs are employed that flank a target region and polymerization is conducted with a strand displacing enzyme.

The present Application claims priority to U.S. Provisional Application Ser. No. 61/388,985 filed Oct. 1, 2010, and U.S. Provisional Application Ser. No. 61/428,652 filed Dec. 30, 2010, the entirety of each of which is herein incorporated by reference.

### STATEMENT REGARDING FEDERALLY SPONSORED RESEARCH OR DEVELOPMENT

This invention was made in part with Government support under Grant Number 1-07-C-0096 awarded by HDTRA, and under Grant Numbers WHIXWH-05-C-0116 and NBCHC 070041 awarded by DHS. The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Oct. 26, 2011, is named 10133W001.txt and is 588,586 bytes in size.

### FIELD OF THE INVENTION

The present invention relates to methods and compositions for targeted genome amplification. In certain embodiments, multiple primer pairs are employed that flank a target region and polymerization is conducted with a strand displacing enzyme.

### BACKGROUND OF THE INVENTION

In many fields of research such as genetic diagnosis, cancer research or forensic medicine, the scarcity of genomic DNA can be a severely limiting factor on the type and quantity of genetic tests that can be performed on a sample. One approach designed to overcome this problem is whole genome amplification. The objective is to amplify a limited DNA sample in a non-specific manner in order to generate a new sample that is indistinguishable from the original but with a higher DNA concentration. The aim of a typical whole genome amplification technique would be to amplify a sample up to a microgram level while respecting the original sequence representation.

The first whole genome amplification methods were described in 1992, and were based on the principles of the polymerase chain reaction. Zhang and coworkers (Zhang, L., et al. Proc. Natl. Acad. Sci. USA, 1992, 89: 5847-5851) developed the primer extension PCR technique (PEP) and Telenius and collaborators (Telenius et al., Genomics. 1992, 13(3):718-25) designed the degenerate oligonucleotide-primed PCR method (DOP-PCR) Zhang et al., 1992). PEP involves a high number of PCR cycles; using Taq polymerase and 15 base random primers that anneal at a low stringency temperature. Although the PEP protocol has been improved in different ways, it still results in incomplete genome coverage, failing to amplify certain sequences such as repeats. Failure to prime and amplify regions containing repeats may lead to incomplete representation of a whole genome because consistent primer coverage across the length of the genome provides for optimal representation of the genome. This method also has limited efficiency on very small samples (such as single cells). Moreover, the use of Taq polymerase implies that the maximal product length is about 3 kb.

DOP-PCR is a method which uses Taq polymerase and semi-degenerate oligonucleotides (such as CGACTCGAGNNNNATGTGG (SEQ ID NO: 1), for example, where N=A, T, C or G) that bind at a low annealing temperature at approximately one million sites within the human genome. The first cycles are followed by a large number of cycles with a higher annealing temperature, allowing only for the amplification of the fragments that were tagged in the first step. This leads to incomplete representation of a whole genome. DOP-PCR generates, like PEP, fragments that are in average 400-500 bp, with a maximum size of 3 kb, although fragments up to 10 kb have been reported. On the other hand, as noted for PEP, a low input of genomic DNA (less than 1 ng) decreases the fidelity and the genome coverage (Kittler et al., Anal. Biochem. 2002, 300(2), 237-44).

Multiple displacement amplification (MDA, also known as strand displacement amplification; SDA) is a non-PCR-based isothermal method based on the annealing of random hexamers to denatured DNA, followed by strand-displacement synthesis at constant temperature (Blanco et al., 1989, J. Biol. Chem. 264:8935-40). It has been applied to small genomic DNA samples, leading to the synthesis of high molecular weight DNA with limited sequence representation bias (Lizardi et al., Nature Genetics 1998, 19, 225-232; Dean et al., Proc. Natl. Acad. Sci. U.S.A. 2002, 99, 5261-5266). As DNA is synthesized by strand displacement, a gradually increasing number of priming events occur, forming a network of hyper-branched DNA structures. The reaction can be catalyzed by the Phi29 DNA polymerase or by the large fragment of the Bst DNA polymerase. The Phi29 DNA polymerase possesses a proofreading activity resulting in error rates 100 times lower than the Taq polymerase.

The methods described above generally produce amplification of whole genomes wherein all of the nucleic acid in a given sample is indiscriminately amplified. These methods cannot selectively amplify target genomes in the presence of background or contaminating genomes. Therefore, the results obtained from these methods have a problematically high amount of contaminating background nucleic acid. Purifying collected samples to isolate target genome(s) and remove background genome(s) will result in a further reduction in the amount of already scarce target genome.

There is a long felt need for a method of targeted amplification of a whole genome relative to background or contaminating genomes. In certain cases where only small quantities of a nucleic acid sample to be tested for the presence of a given target nucleic acid sequence, it would be advantageous to introduce specificity into amplification of whole genomes so that a particular target genome is selectively amplified relative to other genomes present within a given sample. For example, in cases of microbial forensics or clinical diagnostics, it would be useful to selectively amplify a genome of a pathogen, or a class of pathogens relative to the genomes of organisms which are also present in the sample which contains a small quantity of total nucleic acid. This would provide the quantities of nucleic acid of the pathogen that are necessary to identify the pathogen. The methods disclosed herein satisfy this long felt need.

### SUMMARY OF THE INVENTION

In certain embodiments, the present invention provides methods of amplifying a target sequence (e.g., from a pathogen) comprising contacting: a) a sample with a strand displacing polymerase, a first upstream primer, a second upstream primer, a first downstream primer, and a second downstream primer, wherein the sample is suspected of containing a nucleic acid sequence comprising a target region sequence, wherein the first and second upstream primers are able to hybridize to the nucleic acid sequence upstream of the target region sequence, and wherein the first and second downstream primers are able to hybridize to the nucleic acid sequence downstream of the target region sequence; and b) treating same sample under conditions such that: i) a first upstream amplicon is generated comprising the first upstream primer and said target region sequence, ii) a second upstream amplicon is generated that comprises the second upstream primer, the sequence of the first upstream primer, and the target region sequence, wherein the first upstream amplicon is strand displaced by the strand displacing enzyme during the generation of the second upstream amplicon; iii) a first downstream amplicon is generated comprising the first downstream primer and the target region sequence, and iv) a second downstream amplicon is generated that comprises the second downstream primer, the sequence of the first downstream primer, and the target region sequence, wherein the first downstream amplicon is strand displaced by the strand displacing enzyme during the generation of the second downstream amplicon.

In some embodiments, the methods further comprise detecting the presence or absence of the first upstream amplicon, the second upstream amplicon, the first downstream amplicon, the second downstream amplicon, or any combination thereof (e.g., by PCR detection methods). In some embodiments, the treating is incubating the sample under isothermal conditions. In some embodiments, the strand displacing polymerase is a polymerase such as Phi 29, Klenow polymerase, or Bst polymerase. In some embodiments, the strand displacing polymerase is Bst polymerase.

In some embodiments, the sample is a biological sample, an environmental sample, a synthetic sample, or a manufactured sample. In some embodiments, the sample is a biological sample such as blood, serum, plasma, tissue, cells, saliva, sputum, urine, cerebrospinal fluid, pleural fluid, milk, tears, stool, sweat, semen, whole cells, cell constituent, cell smear, or extracts thereof. In some embodiments, the target sequence is present in a spirochete genome. In some embodiments, the spirochete is a member of the genus Borrelia.

In some embodiments, the sample is contacted with at least 3 . . . at least 10 . . . at least 24 . . . or at least 40 upstream primers and at least 3 . . . at least 10 . . . at least 24 . . . or at least 40 downstream primers. In some embodiments, the sample is contacted with at least 50 upstream primers and 50 downstream primers. In some embodiments, the average Tₘ of the primers is in the range of 35-60° C. In some embodiments, the displaced strand of the first upstream amplicon functions as template for amplification by a downstream primer. In some embodiments, the displaced strand of the first downstream amplicon functions as template for amplification by an upstream primer.

In some embodiments, the amplicons are detected using a method such as a PCR method, a mass spectrometry method, or a sequencing method. In some embodiments, the present invention provides a kit for use in conducting methods described herein, the kit comprising at least two upstream primers (e.g., 2 . . . 5 . . . 10 . . . 50 . . . or more), two downstream primers (e.g., 2 . . . 5 . . . 10 . . . 50 . . . or more), and a strand-displacing polymerase. In certain embodiments, the present invention provides a method of amplifying a target sequence comprising: a) contacting a sample with a strand displacing polymerase, at least two upstream primers, and at least two downstream primers, wherein the sample is suspected of containing a nucleic acid sequence comprising a target region, wherein the at least two upstream primers hybridize to the nucleic acid sequence upstream of the target region, and wherein the at least two downstream primers hybridize to the nucleic acid sequence downstream of the target region; and b) treating same sample under conditions such that amplicons are generated from the at least two upstream primer and from the at least two downstream primers.

In some embodiments, the at least two upstream primers comprises at least 3 . . . at least 5 . . . at least 25 . . . or more upstream primers and wherein the at least two downstream primers comprises at least 3 . . . at least 5 . . . at least 25 . . . or more downstream primers. In some embodiments, the strand displacing polymerase is a polymerase such as Phi 29, Klenow polymerase, or Bst polymerase.

Provided herein is an oligonucleotide that is selected by identifying each oligonucleotide of x nucleotides in length that appears in a target genome, where x may be 5-100. A first ratio is calculated by dividing the number of times each oligonucleotide appears in the target genome by the length of the genome in nucleotides. A second ratio or ratios is calculated by dividing the number of times each oligonucloetide appears in one or more background genomes by the length of each respective background genome in nucleotides. The second ratios for each oligonucleotide are summed and divided by the number of background genomes. A combined hit ratio for each oligonucleotide is determined by calculating a ratio of the first ratio to the averaged second ratios. The oligonucleotides are ranked into a list by one or more criteria, which may be by descending order according to the respective combined hit ratio of the oligonucleotides. The oligonucleotide may be selected from the ranked list. The oligonucleotide may be one of the top 600-ranked oligonucleotides from the ranked list, or may be the highest ranked. The oligonucleotide may have a combined hit ratio of at least 5, 10, 20, or 50. Also provided herein is a plurality of oligonucleotides, wherein the oligonucleotides may consist of 2-600 of the ranked oligonucleotides.

Further provided herein is a method for isolating a target genome by providing a sample suspected of comprising the target genome and contacting the sample with the probe. Also provided herein is a method for detecting a target genome by providing a sample suspected of comprising the target genome and contacting the sample with the probe. The presence of bound probe is detected, and the presence of bound probe indicates the presence of the target genome. The method may also comprise performing DNA amplification and detecting the presence of the amplification product, where the presence of the amplified product indicates the presence of the target genome. The sample may comprise a background sequence. The amplification product may consist of a sequence that is contained in a virulence factor gene.

Also provided herein is an oligonucleotide set comprising a plurality of oligonucleotides selected by identifying each oligonucleotide of x nucleotides in length that appears in a target genome, where x may be 5-100. A first ratio is calculated by dividing the number of times each oligonucleotide appears in the target genome by the length of the genome in nucleotides. A second ratio or ratios is calculated by dividing the number of times each oligonucloetide appears in one or more background genomes by the length of each respective background genome in nucleotides. The second ratios for each oligonucleotide are summed and divided by the number of background genomes. A combined hit ratio for each oligonucleotide is determined by calculating a ratio of the first ratio to the averaged second ratios. The oligonucleotides are ranked into a list by one or more criteria, which may be by descending order according to the respective combined hit ratio of the oligonucleotides. A set of oligonucleotides is then generated by an iterative process, which starts with selecting an oligonucleotide from the ranked list with the highest rank and that binds to the target genome at least y times, where y is 0 to 500. It is then determined whether the selected oligonucleotide binds to the target genome within the largest remaining gap in target genome coverage left by oligonucleotides in the primer set. If the oligonucleotide does bind to the target genome within the largest remaining gap in target genome coverage left by the oligonucleotides in the primer set, then the oligonucleotide is added to the primer set. If the oligonucleotide does not bind to the target genome within the largest remaining gap in target genome coverage left by the oligonucleotides in the primer set, then the oligonucleotide is omitted and discarded from the ranked list. The iterative process is repeated 100-600 times. The method for generating the oligonucleotide set may further comprise repeating the iterative process z times to generate z different primer sets, where z is 0-500, and for each iteration, increasing y by 1, and then selecting one of the z primer sets that optimizes the average combined hit ratio for the oligonucleotides in the set and the maximum distance between oligonucleotides of the oligonucleotide set on the target genome.

The oligonucleotide set may be used for targeted whole or partial genome amplification. The background genomes may comprise a human genome, a human mitochondrial genome, a plant genome, or a plant chloroplast genome. The oligonucleotides in the oligonucleotide set may have a combined hit ratio of at least 5, 10, 20 or 50.

Further provided herein is a method of amplifying a target genome by contacting the target genome with the oligonucleotide set and performing DNA amplification. Also provided herein is a method of detecting a pathogen in a patient suspected of being infected by the pathogen by providing a sample isolated from the patient, contacting the sample with the oligonucleotide set, performing DNA amplification, and detecting the presence of the amplification product, where the presence of the amplification product is indicative of the presence of the pathogen. The pathogen may be Borrelia.

Further provided herein is a probe comprising an insert, wherein the insert consists of the sequence of the oligonucleotide selected from the ranked list or from the oligonucleotide set. The probe may be embedded in a gel, which may be a synchronous coefficient of drag alteration (SCODA) method gel. The probe may be attached to a microarray or HPLC. The probe may be a real-time probe, a scorpion probe, a hybridization probe, a 5′-nuclease probe, a molecular beacon probe, or a FISH probe. The oligonucleotide may be selected to identify a part of the genome encoding a virulence factor.

Also provided herein is a kit for performing targeted genome amplification that includes the oligonucleotide set and instructions for using the kit. Further provided herein is a method for detecting the presence of a target genome by providing a sample suspected of containing a target genome that comprises a target sequence, isolating the target genome from a background genome, contacting the target genome with two or more oligonucleotides from the oligonucleotide set, performing DNA amplification and detecting the presence of the amplification product. The presence of the amplification product indicates the presence of the target genome. The isolating may be accomplished by contacting the genome with the SCODA method gel and separating the target genome from the background genome. The amplification product may consist of a sequence that is contained in a virulence factor gene. Further provided herein is a computer system for generating the list of ranked oligonucleotides that includes a process and a memory coupled to the processor. The memory may be configured to store instructions for performing the steps of the method for generating the oligonucleotide set, where the instructions are executed by the processor.

### BRIEF DESCRIPTION OF THE DRAWINGS

FIG. 1 is a plot indicating the relationships between sensitivity, selectivity and length of the genome sequence segments and primers hybridizing thereto.

FIG. 2 is a process diagram indicating the process steps for selection of genome sequence segments and primers hybridizing thereto.

FIG. 3A is a plot indicating the quantities of human DNA obtained from whole genome amplification (WGA) reactions performed with random hexamer primers (solid diamond) and the targeted whole genome amplification (TWGA) method using the primers of Table 3 (clear circle).

FIG. 3B is a plot indicating the quantity of Bacillus anthracis DNA obtained from whole genome amplification (WGA) reactions performed with random hexamer primers (solid diamond) and targeted whole genome amplification (TWGA) method using the primers of Table 3 (clear circle).

FIG. 4A is a plot indicating the quantities of human DNA obtained from whole genome amplification (WGA) reactions performed with random hexamer primers (solid diamond) and the targeted whole genome amplification (TWGA) method using the first generation primers of Table 3 (clear circle) and the second generation primers of Table 4 (clear square).

FIG. 4B is a plot indicating the quantity of Bacillus anthracis DNA obtained from whole genome amplification (WGA) reactions performed with random hexamer primers (solid diamond) and targeted whole genome amplification (TWGA) method using the primers of Table 3 (clear circle) and the second generation primers of Table 4 (clear square).

FIGS. 5A and 5B are plots indicating the quantities of Bacillus anthracis DNA (target genome) and Homo sapiens DNA (background genome) obtained in targeted whole genome amplification reactions with the indicated quantity of background DNA and 200 femtograms (fg) of Bacillus anthracis DNA.

FIGS. 6A and 6B are plots comparing the quantities of Bacillus anthracis DNA (target genome) and Homo sapiens DNA (background genome) obtained in a targeted whole genome amplification reaction (FIG. 6A) vs. a conventional whole genome amplification reaction (FIG. 6B).

FIGS. 7A and 7B are plots of quantity of amplified DNA obtained in a range of concentrations of Bacillus anthracis DNA (target genome) with a constant concentration of Homo sapiens DNA (background genome). FIG. 7A indicates the quantities of Bacillus anthracis DNA obtained in two different targeted whole genome amplification reactions and in a conventional whole genome amplification reaction. FIG. 7B indicates the quantities of Homo sapiens DNA in the same three reactions.

FIG. 8 is a process diagram illustrating a representative primer pair selection process.

FIG. 9 is a process diagram illustrating an embodiment of the calibration method.

FIG. 10 shows the results of targeted genome amplification of Borrelia DNA.

FIG. 11 shows the results of temperature optimization of targeted genome amplification of Borrelia DNA.

FIG. 12 shows the results of incubation time optimization of targeted genome amplification of Borrelia DNA.

FIG. 13 shows the sensitivity of targeted genome amplification of Borrelia DNA.

FIG. 14 is a simplified block diagram of a computer system described herein.

FIG. 15 shows a diagrammatic overview of an embodiment of the present invention using multiple primers and a strand displacing polymerase. Primers are designed to bind flanking the target region (A). As each primer extends a complementary DNA strand is created (B). Since a strand displacement polymerase is used, as a primer upstream creates a strand of DNA it displaces the adjacent downstream strand. This displaced strand can function as a new template for primers in the opposite direction to bind and prime.

FIG. 16 shows specific amplification of K. pneumoniae genome target DNA. DNA extracted from 200 μl human blood was spiked with 20 copies of K. pneumoniae genome, either subjected to TGA amplification or unamplified, followed by quantitative PCR (qPCR) to quantify the K. pneumoniae (16S locus) or Homo sapiens (Hs) Alu regions. TGA reaction amplified the K. pneumoniae 16S region over 25-fold, despite a 6,000,000-fold excess of human DNA.

FIG. 17 shows amplification of K. pneumoniae genome 149-fold (primer extension pair A) or 66-fold (primer extension pair B), respectively. DNA extracted from 200 μl human blood was spiked with 20 copies of K. pneumoniae genome, either subjected to TGA amplification or unamplified, followed by T5000 quantification.

FIG. 18 shows that no K. pneumoniae genome target DNA (16S or 23S loci) was detected by ESI-MS analysis of reaction components.

FIG. 19 shows the results of testing additional TGA primer sets to detect B. burgdorferi genome target DNA. Fifty copies of B. burgdorferi genome were added to 200 μl of human DNA extracted from 1 ml blood. The indicated primer sets were used at indicated concentrations for TGA, where the TGA incubation was conducted for 4 hours at their indicated annealing temperature, followed by incubation at 80° C. for 20 minutes and hold at 4° C. Two microliters of each TGA reaction was analyzed by ESI-MS. All primer sets were directed towards target 3511.

FIG. 20 shows the results of testing additional TGA primer sets to detect B. burgdorferi genome target DNA. Fifty copies of B. burgdorferi genome were added to 200 μl of human DNA extracted from 1 ml blood. The indicated primer sets were used at indicated concentrations for TGA, where the TGA incubation was conducted for 4 hours at their indicated annealing temperature, followed by incubation at 80° C. for 20 minutes and hold at 4° C. Five microliters of each TGA reaction was analyzed by ESI-MS. Primer sets directed towards loci 3517, 3514, or 3511 were used, respectively. Primer set “E3 mix” included 25 pairs of forward and reverse primers for each locus covering all three loci (75 primer pairs total, or 150 individual primers). Primer set “3p mix” includes the original “E set” primers (12 forward and 13 reverse primers for each of loci 3517, 3514, and 3511, for a total of 75 primers.)

FIG. 21 shows the results of testing additional TGA primer sets to detect B. burgdorferi genome target DNA. Thirty copies of B. burgdorferi genome were added to 200 μl of human DNA extracted from 1 ml blood. The indicated primer sets were used at indicated concentrations for TGA, where the TGA incubation was conducted for 4 hours at 56° C., followed by incubation at 80° C. for 20 minutes and hold at 4° C. Samples were treated with calf intestinal phosphatase (CIP), then 5 μA of each sample was loaded per well of a Borrelia MLST (multi-locus sequence typing) genotyping plate, cycled on an Eppendorf procycler thermocycler, and analyzed on a PlexID unit. Primer set “E3 mix” included 25 pairs of forward and reverse primers for each locus covering all three loci (75 primer pairs total, or 150 individual primers). Primer set “8E3” includes the “E3 set” primers, plus additional primers covering loci 3519-20, 3516, 3515, and 3518, where the 3519-20 primers covered two loci in total (loci 3519 and 3520), allowing the 8E3 primer set to cover eight loci altogether. Accordingly, the 8E3 primer set included the E3 set (12 forward and 13 reverse primers for each of loci 3517, 3514, and 3511, for a total of 75 primers) as well as 25 primer sets (25 forward primers and 25 reverse primers) for each of 3519-20, 3516, 3515, and 3518 (a total of 200 new primers).

### DETAILED DESCRIPTION

In certain embodiments, the present invention provides methods for amplifying trace amounts of specific DNA targets in samples that contain large amounts of other DNA. In general, these methods revolves around using multiple oligonucleotide primers flanking the target sequence in an isothermal nested amplification reaction.

As shown in the Examples below, the use of this method has been demonstrated by amplifying trace amounts of broad range 16s and 23s DNA targets and specific Borrelia DNA targets in samples containing large amounts of human DNA. Primers were designed to selectively amplify 16s and 23s targets of bacterial genomes as well as different regions of Borrelia DNA to increase the total copies for PCR detection. The PCR product was then subjected to ESI-MS for the detection of the 16s and 23s DNA targets or Borrelia DNA targets.

Many DNA targets for PCR detection are at extremely low copies and in the presence of other DNA targets. By selectively increasing the number of target DNA, the detection sensitivity of PCR can be increased without increasing background. The Examples below using 16s and 23s DNA targets are important because even during a significant septic infection, the number of bacterial genome copies in blood can be extremely low making detection of the pathogen unreliable. Using the TGA methods described herein, one can selectively increase the amount of 16s and 23s DNA allowing for reliable detection of the bacteria in isolates with trace samples of bacterial DNA.

The Example below using Borrelia is important because even in patients during the acute phase of Lyme disease the number of spirochetes in the blood is extremely low making PCR detection of the pathogen unreliable. Using the TGA methods of the present invention, one is able to selectively increase the amount of Borrelia allowing for reliable detection of Borrelia in isolates with trace samples of Borrelia DNA.

One advantage of the methods of the present invention is that they allow for the detection of very trace amounts of the target DNA even when they are in the presence of a large amount of background DNA. For example in bacterial infections when the amount of bacteria in the blood is low the methods allow for the selective amplification of the bacterial target even in the presence of human DNA from the blood.

Since the methods of the present invention are selective in their amplification, they have advantages over whole genome amplification (WGA) strategies as WGA amplifies all of the DNA present increasing the background DNA along with the target of interest DNA. For example, one of the advantages of embodiments of the methods of the present invention is by selectively amplifying 16s, 23, or Borrelia DNA one can reliably detect the 16s, 23s, or Borrelia DNA even when it is in trace amounts and in the presence of overwhelming amounts of other DNA such as in a blood sample. A reliable PCR test for 16s, 23s, or Borrelia targets can provide quick and accurate detection of the pathogen in samples where it was previously too low to detect.

The inventors have made the surprising discovery that whole or parts of target genomes, such as microbial genomes, can be selectively amplified from a mixture of target and background DNA, such as host nuclear DNA and host mitochondrial DNA. This amplification can be accomplished by using oligonucleotide sets that include fewer oligonucleotides that preferentially bind to sequences that are highly repeated throughout the target genome, but appear only rarely in the background DNA. The oligonucleotides are selected to maximize the number of locations in the target genome to which they can bind in proportion to the target genome size, as compared to the average of the number of locations within the background DNAs in proportion to the respective background DNAs. Accordingly, the oligonucleotides of the oligonucleotide set are chosen to have a high degree of selectivity for the target genome in comparison to background DNA. The oligonucleotides of the oligonucleotide set are further selected in order to balance high target genome selectivity with providing the greatest amount of target genome coverage, with as short a distance between oligonucleotide binding sites in the target genome as possible.

Particular oligonucleotides can be further selected from the oligonucleotide sets for amplifying only specific regions of interest within the target genome, rather than the entire genome. Such oligonucleotides are more sensitive than oligonucleotides known in the art, because oligonucleotides from the oligonucleotide set are more selective for the target genome as compared to the background genomes. For example, the particular oligonucleotides can be used to amplify a gene that encodes for a virulence factor of a pathogen containing the target genome.

Thus, the targeted partial and whole genome oligonucleotide sets disclosed herein are designed to be far more sensitive than other known techniques; the oligonucleotide sets are capable of amplifying target microbial genomic DNA that is present in very small quantities in comparison to background host DNAs. The amounts of target DNA that can be detected using the instantly disclosed oligonucleotide sets approach the limits of detection for targets, for example, ten bacteria in 1 mL of blood. The instantly disclosed oligonucleotide sets generally only need to include 100-600 different primers of approximately 7-12 nucleotides in length, although other lengths are possible. Fewer oligonucleotides, and as few as two, are used for partial target genome amplification.

In addition, the oligonucleotides of the oligonucleotide sets disclosed herein can be used as capture probes for isolating a target genome from a sample that may also contain the background genome(s). The oligonucleotides of the oligonucleotide set are more selective for the target genome as compared to other capture probes known in the art.

### 1. DEFINITIONS

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number is explicitly contemplated with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the term “abundance” refers to an amount. The amount may be described in terms of concentration, which are common in molecular biology such as “copy number” “pfu or plate-forming unit,” and are well known to those with ordinary skill. Concentration may be relative to a known standard or may be absolute.

The term “amplification,” as used herein, refers to a process of multiplying an original quantity of a nucleic acid template in order to obtain greater quantities of the original nucleic acid.

As used herein, the term “amplifiable nucleic acid” is used in reference to nucleic acids that may be amplified by any amplification method. It is contemplated that “amplifiable nucleic acid” also applies to the term “sample template.”

As used herein, the term “amplification reagents” refers to those reagents (deoxyribonucleotide triphosphates, buffer, etc.), needed for amplification, excluding primers, nucleic acid template, and the amplification enzyme. Typically, amplification reagents along with other reaction components are placed and contained in a reaction vessel (test tube, micro-well, or other vessel).

As used herein, the term “analogous” when used in context of comparison of bioagent identifying amplicons indicates that the bioagent identifying amplicons being compared are produced with the same pair of primers. For example, bioagent identifying amplicon “A” and bioagent identifying amplicon “B”, produced with the same pair of primers are analogous with respect to each other. Bioagent identifying amplicon “C”, produced with a different pair of primers is not analogous to either bioagent identifying amplicon “A” or bioagent identifying amplicon “B”.

As used herein, the term “anion exchange functional group” refers to a positively charged functional group capable of binding an anion through an electrostatic interaction. The most well known anion exchange functional groups are the amines, including primary, secondary, tertiary and quaternary amines.

The term “background organisms,” as used herein, refers to organisms typically present in a given sample that are not of interest and are thus considered to be contaminants. The background organism may be a pathogen, a virus, a bacterium, a protozoan, or a multicellular organism such as a fungus, plant, algae, or animal, or any other kind of bioagent.

The term “background genome,” as used herein refers to the DNA of a background organism, such as the genome of the organism. Background organisms will vary according to the sample source. In a non-limiting example, for targeted genome amplification of a soil bioremediation bacterium in a soil sample, it would be advantageous to define the genomes of organisms native to soil such as C. elegans, as background genomes. In another non-limiting example, for whole genome amplification of a genome belonging to a target pathogen in a human tissue sample, it would be advantageous to define human nuclear DNA, and optionally human mitochondrial DNA, as a background genome. The background genome may be a plasmid. The background genome may also be an organellar genome such as that of a mitochondrion or chloroplasts.

The term “bacteria” or “bacterium” refers to any member of the groups of eubacteria and archaebacteria.

The term “bacteremia” refers to the presence of bacteria in the bloodstream. It is also known by the related terms “blood poisoning” or “toxemia.” In the hospital, indwelling catheters are a frequent cause of bacteremia and subsequent nosocomial infections, because they provide a means by which bacteria normally found on the skin can enter the bloodstream. Other causes of bacteremia include dental procedures (occasionally including simple tooth brushing), herpes (including herpetic whitlow), urinary tract infections, intravenous drug use, and colorectal cancer. Bacteremia may also be seen in oropharyngeal, gastrointestinal or genitourinary surgery or exploration.

As used herein, a “base composition” is the exact number of each nucleobase (for example, A, T, C and G) in a segment of nucleic acid. For example, amplification of nucleic acid of strain 5170 of Mycobacterium tuberculosis using primer pair number 3550 (SEQ ID NOs: 673:697) produces an amplification product 129 nucleobases in length from nucleic acid of the embB gene that has a base composition of A21 G37 C44 T27 (by convention—with reference to the sense strand of the amplification product). Because the molecular masses of each of the four natural nucleotides and chemical modifications thereof are known (if applicable), a measured molecular mass can be deconvoluted to a list of possible base compositions. Identification of a base composition of a sense strand which is complementary to the corresponding antisense strand in terms of base composition provides a confirmation of the true base composition of an unknown amplification product. For example, the base composition of the antisense strand of the 129 nucleobase amplification product described above is A27 G44 C37 T21.

As used herein, a “base composition probability cloud” is a representation of the diversity in base composition resulting from a variation in sequence that occurs among different isolates of a given species. The “base composition probability cloud” represents the base composition constraints for each species and is typically visualized using a pseudo four-dimensional plot.

As used herein, a “bioagent” is any organism, cell, or virus, living or dead, or a nucleic acid derived from such an organism, cell or virus. The bioagent may contain a target genome or a background genome. Examples of bioagents include, but are not limited, to cells, (including but not limited to human clinical samples, bacterial cells and other pathogens), viruses, fungi, protists, parasites, and pathogenicity markers (including but not limited to: pathogenicity islands, antibiotic resistance genes, virulence factors, toxin genes and other bioregulating compounds). Samples may be alive or dead or in a vegetative state (for example, vegetative bacteria or spores) and may be encapsulated or bioengineered. As used herein, a “pathogen” is a bioagent which causes a disease or disorder. A pathogen that infects a human is known as a “human pathogen.” Non-human pathogens may infect specific animals but not humans. Human pathogens are of interest for clinical reasons and non-human pathogen identification is of interest in veterinary applications of the methods disclosed herein.

As used herein, a “bioagent division” is defined as group of bioagents above the species level and includes but is not limited to, orders, families, classes, clades, genera or other such groupings of bioagents above the species level.

As used herein, the term “bioagent identifying amplicon” refers to a polynucleotide that is amplified from nucleic acid of a bioagent in an amplification reaction and which 1) provides sufficient variability to distinguish among bioagents from whose nucleic acid the bioagent identifying amplicon is produced and 2) whose molecular mass is amenable to a rapid and convenient molecular mass determination modality such as mass spectrometry, for example. In silico representations of bioagent identifying amplicons are particularly useful for inclusion in databases used for identification of bioagents. Bioagent identifying amplicons are defined by a pair of primers that hybridize to regions of nucleic acid of a given bioagent. The bioagent identifying amplicon may be unique to a bioagent containing a target genome. A bioagent containing a target genome may be distinguishable from a bioagent containing a background genome.

As used herein, the term “biological product” refers to any product originating from an organism. Biological products are often products of processes of biotechnology. Examples of biological products include, but are not limited to: cultured cell lines, cellular components, antibodies, proteins and other cell-derived biomolecules, growth media, growth harvest fluids, natural products and bio-pharmaceutical products.

The terms “biowarfare agent” and “bioweapon” are synonymous and refer to a bacterium, virus, fungus or protozoan that could be deployed as a weapon to cause bodily harm to individuals. Military or terrorist groups may be implicated in deployment of biowarfare agents. As used herein, the term “broad range survey primer pair” refers to a primer pair designed to produce bioagent identifying amplicons across different broad groupings of bioagents. For example, the ribosomal RNA-targeted primer pairs are broad range survey primer pairs which have the capability of producing bacterial bioagent identifying amplicons for essentially all known bacteria. With respect to broad range primer pairs employed for identification of bacteria, a broad range survey primer pair for bacteria such as 16S rRNA primer pair number 346 (SEQ ID NOs: 594:602) for example, will produce an bacterial bioagent identifying amplicon for essentially all known bacteria. The broad range survey primer pair may bind to target genome sequence segments within the target genomes of the broad grouping of bioagents.

The term “calibration amplicon” refers to a nucleic acid segment representing an amplification product obtained by amplification of a calibration sequence with a pair of primers designed to produce a bioagent identifying amplicon.

The term “calibration sequence” refers to a polynucleotide sequence to which a given pair of primers hybridizes for the purpose of producing an internal (i.e., included in the reaction) calibration standard amplification product for use in determining the quantity of a bioagent in a sample. The calibration sequence may be expressly added to an amplification reaction, or may already be present in the sample prior to analysis.

The term “Glade primer pair” refers to a primer pair designed to produce bioagent identifying amplicons for species belonging to a Glade group. A Glade primer pair may also be considered as a “speciating” primer pair which is useful for distinguishing among closely related species.

The term “codon” refers to a set of three adjoined nucleotides (triplet) that codes for an amino acid or a termination signal.

As used herein, the term “codon base composition analysis,” refers to determination of the base composition of an individual codon by obtaining a bioagent identifying amplicon that includes the codon. The bioagent identifying amplicon will at least include regions of the target nucleic acid sequence to which the primers hybridize for generation of the bioagent identifying amplicon as well as the codon being analyzed, located between the two primer hybridization regions.

As used herein, the terms “complementary” or “complementarity” are used in reference to polynucleotides (i.e., a sequence of nucleotides such as an oligonucleotide or a target nucleic acid) related by the base-pairing rules. For example, the sequence 5′-A-G-T-3′, is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand. But in this sense, complementarity either exists or does not exist i.e.: there is no partial complementarity.

The term “complement of a nucleic acid sequence” as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids disclosed herein and include, for example, inosine and 7-deazaguanine Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. Where a first oligonucleotide is complementary to a region of a target nucleic acid and a second oligonucleotide has complementary to the same region (or a portion of this region) a “region of overlap” exists along the target nucleic acid. The degree of overlap will vary depending upon the extent of the complementarity.

The term “degenerate primers,” as used herein refers to a mixture of similar, but not identical, primers having one or more residues substituted relative to the other primer(s) in the mixture. Degenerate nucleotide codes include R, K, S, Y, M, W, B, H, N, D, V and I. The corresponding combinations are listed in 37 CFR §1.821. For example, the sequence AAATTTRCCCGGG (SEQ ID NO: 2) actually refers to a combination of primers having the following sequences: AAATTTACCCGGG (SEQ ID NO: 3), and AAATTTGCCCGGG (SEQ ID NO: 4) because R=A or G.

As used herein, the term “division-wide primer pair” refers to a primer pair designed to produce bioagent identifying amplicons within sections of a broader spectrum of bioagents. The division-wide primer pair may bind to target genome sequence segments within target genomes of the broader spectrum of bioagents. For example, primer pair number 354 (SEQ ID NOs: 597:605), a division-wide primer pair, is designed to produce bacterial bioagent identifying amplicons for members of the Bacillus group of bacteria which comprises, for example, members of the genera Streptococcus, Enterococcus, and Staphylococcus. Other division-wide primer pairs may be used to produce bacterial bioagent identifying amplicons for other groups of bacterial bioagents.

As used herein, the term “concurrently amplifying” used with respect to more than one amplification reaction refers to the act of simultaneously amplifying more than one nucleic acid in a single reaction mixture.

As used herein, the term “drill-down primer pair” refers to a primer pair designed to produce bioagent identifying amplicons for identification of sub-species characteristics or confirmation of a species assignment. For example, primer pair number 897 (SEQ ID NOs: 717:727), a drill-down Staphylococcus aureus genotyping primer pair, is designed to produce Staphylococcus aureus genotyping amplicons. Other drill-down primer pairs may be used to produce bioagent identifying amplicons for Staphylococcus aureus and other bacterial species. The term “duplex” refers to the state of nucleic acids in which the base portions of the nucleotides on one strand are bound through hydrogen bonding to their complementary bases arrayed on a second strand. The condition of being in a duplex form reflects on the state of the bases of a nucleic acid. By virtue of base pairing, the strands of nucleic acid also generally assume the tertiary structure of a double helix, having a major and a minor groove. The assumption of the helical form is implicit in the act of becoming duplexed.

As used herein, the term “etiology” refers to the causes or origins, of diseases or abnormal physiological conditions.

The term “frequency of occurrence” as used herein, refers to the number of different coordinates where a given genome sequence segment occurs within a given genome. The frequency of occurrence of a given genome sequence segment provides a means of defining the sensitivity of a primer designed to hybridize to the genome sequence segment. The frequency of occurrence of a given genome sequence segment is also used in the calculation of selectivity ratios, hit ratios, and combined hit ratios.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or function is retained.

The term “genome,” as used herein, generally refers to the complete set of genetic information in the form of one or more nucleic acid sequences, including text or in silico representations thereof. A genome may include either DNA or RNA, depending upon its organism of origin. Most organisms have DNA genomes while some viruses have RNA genomes. As used herein, the term “genome” need not comprise the complete set of genetic information. The term may also refer to at least a majority portion of a genome such as at least 50% to 100% of an entire genome or any whole or fractional percentage therebetween. The term genome may also refer to part of a genome. For example, the genome may be a chromosome, or a portion of a chromosome. The genome may also not be a contiguous segment of DNA. The genome may be a number of regions with a common characteristic, such as including coding regions that encode similar types of products, or such as including RNA genes. For example, the genome may be portions that contain ribosome-producing sequences. The part of the genome may be targeted in order to detect a particular genome or to make a diagnosis, such as of an infection by a pathogen.

The term “genome sequence segment,” as used herein, refers to a portion of a genome sequence which is initially defined as a primer hybridization candidate for the purpose of the targeted genome amplification methods disclosed herein. The genome sequence segment may be 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length. The related term “unique genome sequence segment” refers to a genome sequence segment that occurs at least once in a given genome. For example, a simplified hypothetical 8 nucleobase genome consisting of the following sequence: aattccgg (SEQ ID NO: 5) has four unique genome sequence segments of five nucleobase lengths (aattc (SEQ ID NO: 6); attcc (SEQ ID NO: 7); ttccg (SEQ ID NO: 8); and tccgg (SEQ ID NO: 9)). This same simplified hypothetical 8 nucleobase genome also has three unique genome sequence segments of six nucleobase lengths: (aattcc (SEQ ID NO: 10); attccg (SEQ ID NO: 11); and ttccgg (SEQ ID NO: 12)). This same simplified hypothetical 8 nucleobase genome also has two unique genome sequence segments of seven nucleobase lengths: (aattccg (SEQ ID NO: 13); and attccgg (SEQ ID NO: 14)). This same simplified hypothetical 8 nucleobase genome also has one unique genome sequence segment which is 8 nucleobases in length: (aattccgg (SEQ ID NO: 5). In another example, a simplified hypothetical 8 nucleobase genome consisting of the following sequence: aaaaaaaa (SEQ ID NO: 15) obviously only has a single unique genome sequence segment which is five nucleobases in length (occurring 4 times), as well as a single unique genome sequence segment which is six nucleobases in length (occurring 3 times), a single unique genome sequence segment which is seven nucleobases in length (occurring twice) and a single unique genome sequence segment which is eight nucleobases in length (occurring once).

The term “genotype,” as used herein, refers to the genetic makeup of an organism. Members of the same species of organism having genetic differences are said to have different genotypes.

The term “hit ratio” as used herein, refers to a variable calculated by determining the frequency of occurrence of a given genome sequence segment within the target genome divided by the length of the given genome, and then dividing this by the frequency of occurrence of the given genome sequence segment in a background genome divided by the length of the background genome. For example, if there is one target genome (A) and one background genome (B), and the frequency of occurrence for the given genome sequence segment is 1 in A and B, the hit ratio would be calculated as follows:

(1(A)/length of genome A)/(1(B)/length of genome B)

If the hit ratio is being calculated for a target genome that is less than an entire genome, such as a chromosome or a portion of a chromosome, then the frequency of occurrence of a given genome sequence segment would be determined for the chromosome or portion of the chromosome. The frequency of occurrence would then be divided by the length of the chromosome or portion of the chromosome. Additionally, the remainder of the genome that is not included in the target genome becomes a background genome for determining the hit ratio. The hit ratio would otherwise be calculated as above.

Similarly, when there is more than one background genome, a “combined hit ratio” can be calculated. The term “combined hit ratio” as used herein, refers to a variable calculated by determining the frequency of occurrence of a given genome sequence segment within the target genome divided by the length of the target genome, and then dividing this by the average of the frequency of occurrence of the given genome sequence segment within each background genome divided by the length of the respective background genome. For example, if there is one target genome (A) and two background genomes (B and C, respectively), such as nuclear genomic DNA (B) and mitochondrial DNA (C), and the frequency of occurrence for the given genome sequence segment is 1 in A, B, and C, then the combined hit ratio would be calculated as follows:

(1(A)/length of genome A)/((1(B)/length of genome B)+(1(C)/length of genome C)/2)

If the combined hit ratio is being determined for a target genome that does not include an entire genome, such as a chromosome or a portion of a chromosome, then the frequency of occurrence of a given genome sequence segment would be determined for the chromosome or portion of the chromosome. The frequency of occurrence would then be divided by the length of the chromosome or portion of the chromosome. Additionally, the remainder of the genome that is not included in the target genome becomes a background genome for determining the combined hit ratio. The combined hit ratio would otherwise be calculated as above. The combined hit ratio may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater.

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer nucleobases in length would have 15/20=0.75 or 75% sequence identity with the 20 nucleobase primer. As used herein, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers disclosed herein, sequence identity is properly determined when the alignment is designated as Plus/Plus. Sequence identity may also encompass alternate or modified nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, “housekeeping gene” refers to a gene encoding a protein or RNA involved in basic functions required for survival and reproduction of a bioagent. Housekeeping genes include, but are not limited to genes encoding RNA or proteins involved in translation, replication, recombination and repair, transcription, nucleotide metabolism, amino acid metabolism, lipid metabolism, energy generation, uptake, secretion and the like.

The term “hybridization,” as used herein refers to the process of joining two complementary strands of DNA or one each of DNA and RNA to form a double-stranded molecule.

The term “in silico” refers to processes taking place via computer calculations. For example, electronic PCR (ePCR) is a process analogous to ordinary PCR except that it is carried out using nucleic acid sequences and primer pair sequences stored on a computer formatted medium.

The term “in vitro method,” as used herein, describes a biochemical process performed in a test-tube or other laboratory apparatus. An amplification reaction performed on a nucleic acid sample in a microtube or a well of a multi-well plate is an example of an in vitro method. The “ligase chain reaction” (LCR; sometimes referred to as “Ligase Amplification Reaction” (LAR) described by Barany, Proc. Natl. Acad. Sci., 88:189 (1991); Barany, PCR Methods and Applic., 1:5 (1991); and Wu and Wallace, Genomics 4:560 (1989) has developed into a well-recognized alternative method for amplifying nucleic acids. In LCR, four oligonucleotides, two adjacent oligonucleotides which uniquely hybridize to one strand of target DNA, and a complementary set of adjacent oligonucleotides, that hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, hybridization and ligation amplify a short segment of DNA. LCR has also been used in combination with PCR to achieve enhanced detection of single-base changes. However, because the four oligonucleotides used in this assay can pair to form two short ligatable fragments, there is the potential for the generation of target-independent background signal. The use of LCR for mutant screening is limited to the examination of specific nucleic acid positions.

The term “locked nucleic acid” or “LNA” refers to a nucleic acid analogue containing one or more 2′-O, 4′-C-methylene-.beta.-D-ribofuranosyl nucleotide monomers in an RNA mimicking sugar conformation. LNA oligonucleotides display unprecedented hybridization affinity toward complementary single-stranded RNA and complementary single- or double-stranded DNA. LNA oligonucleotides induce A-type (RNA-like) duplex conformations. The primers disclosed herein may contain LNA modifications.

As used herein, the term “mass-modifying tag” refers to any modification to a given nucleotide which results in an increase in mass relative to the analogous non-mass modified nucleotide. Mass-modifying tags can include heavy isotopes of one or more elements included in the nucleotide such as carbon-13 for example. Other possible modifications include addition of substituents such as iodine or bromine at the 5 position of the nucleobase for example.

The term “mass spectrometry” refers to measurement of the mass of atoms or molecules. The molecules are first converted to ions, which are separated using electric or magnetic fields according to the ratio of their mass to electric charge. The measured masses are used to identity the molecules.

The term “mean” as used herein refers to the arithmetic average; the sum of the data divided by the sample size.

The term “microorganism” as used herein means an organism too small to be observed with the unaided eye and includes, but is not limited to bacteria, virus, protozoans, fungi; and ciliates.

The term “multi-drug resistant” or multiple-drug resistant” refers to a microorganism which is resistant to more than one of the antibiotics or antimicrobial agents used in the treatment of said microorganism.

The term “multiplex PCR” refers to a PCR reaction where more than one primer set is included in the reaction pool allowing 2 or more different DNA targets to be amplified by PCR in a single reaction tube.

The term “non-template tag” refers to a stretch of at least three guanine or cytosine nucleobases of a primer used to produce a bioagent identifying amplicon which are not complementary to the template. A non-template tag is incorporated into a primer for the purpose of increasing the primer-duplex stability of later cycles of amplification by incorporation of extra G-C pairs which each have one additional hydrogen bond relative to an A-T pair.

The term “nucleic acid sequence” as used herein refers to the linear composition of the nucleic acid residues A, T, C or G or any modifications thereof, within an oligonucleotide, nucleotide or polynucleotide, and fragments or portions thereof, and to DNA or RNA of genomic or synthetic origin which may be single or double stranded, and represent the sense or antisense strand

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

The term “nucleotide analog” as used herein refers to modified or non-naturally occurring nucleotides such as 5-propynyl pyrimidines (i.e., 5-propynyl-dTTP and 5-propynyl-dTCP), 7-deaza purines (i.e., 7-deaza-dATP and 7-deaza-dGTP). Nucleotide analogs include base analogs and comprise modified forms of deoxyribonucleotides as well as ribonucleotides.

The term “oligonucleotide” as used herein is defined as a molecule comprising two or more deoxyribonucleotides or ribonucleotides, preferably at least 5 nucleotides, more preferably at least about 13 to 35 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof. Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′-end” if its 5′ phosphate is not linked to the 3′ oxygen of a mononucleotide pentose ring and as the “3′-end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction. All oligonucleotide primers disclosed herein are understood to be presented in the 5′ to 3′ direction when reading left to right. When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “organism,” as used herein, refers to humans, animals, plants, protozoa, bacteria, fungi and viruses.

As used here, a “partial genome,” may refer to any portion of a genome that is less than 100%. The partial genome may be a particular chromosome, a plasmid, a gene cluster, a gene, or a polymorphic region, or any other portion of interest of a genome.

As used herein, a “pathogen” is a bioagent which causes a disease or disorder.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “peptide nucleic acid” (“PNA”) as used herein refers to a molecule comprising bases or base analogs such as would be found in natural nucleic acid, but attached to a peptide backbone rather than the sugar-phosphate backbone typical of nucleic acids. The attachment of the bases to the peptide is such as to allow the bases to base pair with complementary bases of nucleic acid in a manner similar to that of an oligonucleotide. These small molecules, also designated anti gene agents, stop transcript elongation by binding to their complementary strand of nucleic acid (Nielsen, et al. Anticancer Drug Des. 1993, 8, 53-63). The primers disclosed herein may comprise PNAs.

The term “polymerase” refers to an enzyme having the ability to synthesize a complementary strand of nucleic acid from a starting template nucleic acid strand and free dNTPs.

As used herein, the term “polymerase chain reaction” (“PCR”) refers to the method of K. B. Mullis U.S. Pat. Nos. 4,683,195, 4,683,202, and 4,965,188, the contents of which are incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The term “polymerization means” or “polymerization agent” refers to any agent capable of facilitating the addition of nucleoside triphosphates to an oligonucleotide. Preferred polymerization means comprise DNA and RNA polymerases.

The term “primer,” as used herein refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. The primer may be an oligodeoxyribonucleotide. The primer must be sufficiently long to prime the synthesis of extension products in the presence of the inducing agent. The exact lengths of the primers will depend on many factors, including temperature, source of primer, use of the method, and the parameters used for primer design, as disclosed herein. A primer may be less than 100% complementary to its corresponding original genome sequence segment. For example, the primer may be 70%, 75%, 80%, 85%, 90%, or 95% complementary to its corresponding original genome sequence segment.

As used herein, the terms “pair of primers,” or “primer pair” are synonymous. A primer pair is used for amplification of a nucleic acid sequence. A pair of primers comprises a forward primer and a reverse primer. The forward primer hybridizes to a sense strand of a target gene sequence to be amplified and primes synthesis of an antisense strand (complementary to the sense strand) using the target sequence as a template. A reverse primer hybridizes to the antisense strand of a target gene sequence to be amplified and primes synthesis of a sense strand (complementary to the antisense strand) using the target sequence as a template.

The primer pairs are designed to bind to highly conserved sequence regions of a bioagent identifying amplicon that flank an intervening variable region and yield amplification products which ideally provide enough variability to distinguish each individual bioagent, and which are amenable to molecular mass analysis. In some embodiments, the highly conserved sequence regions exhibit between about 80-100%, or between about 90-100%, or between about 95-100% identity, or between about 99-100% identity. The molecular mass of a given amplification product provides a means of identifying the bioagent from which it was obtained, due to the variability of the variable region. Thus design of the primers requires selection of a variable region with appropriate variability to resolve the identity of a given bioagent. Bioagent identifying amplicons are ideally specific to the identity of the bioagent.

Properties of the primers may include any number of properties related to structure including, but not limited to: nucleobase length which may be contiguous (linked together) or non-contiguous (for example, two or more contiguous segments which are joined by a linker or loop moiety), modified or universal nucleobases (used for specific purposes such as for example, increasing hybridization affinity, preventing non-templated adenylation and modifying molecular mass) percent complementarity to a given target sequences.

Properties of the primers also include functional features including, but not limited to, orientation of hybridization (forward or reverse) relative to a nucleic acid template. The coding or sense strand is the strand to which the forward priming primer hybridizes (forward priming orientation) while the reverse priming primer hybridizes to the non-coding or antisense strand (reverse priming orientation). The functional properties of a given primer pair also include the generic template nucleic acid to which the primer pair hybridizes. For example, in the case of primer pairs, identification of bioagents can be accomplished at different levels using primers suited to resolution of each individual level of identification. Broad range survey primers are designed with the objective of identifying a bioagent as a member of a particular division (e.g., an order, family, genus or other such grouping of bioagents above the species level of bioagents). In some embodiments, broad range survey intelligent primers are capable of identification of bioagents at the species or sub-species level. Other primers may have the functionality of producing bioagent identifying amplicons for members of a given taxonomic genus, lade, species, sub-species or genotype (including genetic variants which may include presence of virulence genes or antibiotic resistance genes or mutations). Additional functional properties of primer pairs include the functionality of performing amplification either singly (single primer pair per amplification reaction vessel) or in a multiplex fashion (multiple primer pairs and multiple amplification reactions within a single reaction vessel).

The term “processivity,” as used herein, refers to the ability of an enzyme to repetitively continue its catalytic function without dissociating from its substrate. For example, Phi29 polymerase is a highly processive polymerase due to its tight binding of the template DNA substrate.

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

The term “reverse transcriptase” refers to an enzyme having the ability to transcribe DNA from an RNA template. This enzymatic activity is known as reverse transcriptase activity. Reverse transcriptase activity is desirable in order to obtain DNA from RNA viruses which can then be amplified and analyzed by the methods disclosed herein.

The term “ribosomal RNA” or “rRNA” refers to the primary ribonucleic acid constituent of ribosomes. Ribosomes are the protein-manufacturing organelles of cells and exist in the cytoplasm. Ribosomal RNAs are transcribed from the DNA genes encoding them.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc. Environmental samples include environmental material such as surface matter, soil, water, air and industrial samples, as well as samples obtained from food and dairy processing instruments, apparatus, equipment, utensils, disposable and non-disposable items. These examples are not to be construed as limiting the sample types applicable to the methods disclosed herein. The term “source of target nucleic acid” refers to any sample that contains nucleic acids (RNA or DNA). Particularly preferred sources of nucleic acids are biological samples including, but not limited to blood, saliva, urine, cerebral spinal fluid, pleural fluid, milk, lymph, sputum and semen. In particular, different fractions of blood samples exist such as serum or plasma (the liquid component of blood which contains various vital proteins), and buffy coat (a centrifuged fraction of blood that contains white blood cells and platelets). Other preferred sources of nucleic acids are specific cell types such as, hepatic cells for example. Other preferred sources of nucleic acids are tissue biopsies. Methods of handing such samples are well within the technical skill of an ordinary practitioner in the art.

As used herein, the term “sample template” refers to nucleic acid originating from a sample that is analyzed for the presence of “target” (defined below). In contrast, “background template” is used in reference to nucleic acid other than sample template that may or may not be present in a sample. Background template is often a contaminant. It may be the result of carryover, or it may be due to the presence of nucleic acid contaminants sought to be purified away from the sample. For example, nucleic acids from organisms other than those to be detected may be present as background in a test sample.

A “segment” is defined herein as a region of nucleic acid within a nucleic acid sequence. The term “selectivity,” as used herein, is a measure which indicates the frequency of occurrence of a given genome sequence segment in a target relative to the frequency of occurrence of the same genome sequence segment in background genomes. The related term “selectivity ratio,” as used herein, is a number calculated by dividing the frequency of occurrence of a given genome sequence segment in a target genome by its frequency of occurrence in background genomes. Selectivity may also be measured as a hit ratio or combined hit ratio as described herein.

The “self-sustained sequence replication reaction” (3SR) (Guatelli et al., Proc. Natl. Acad. Sci. 1990, 87:1874-1878, with an erratum at Proc. Natl. Acad. Sci. 1990, 87:7797) is a transcription-based in vitro amplification system (Kwok et al., Proc. Natl. Acad. Sci. 1989, 86:1173-1177) that can exponentially amplify RNA sequences at a uniform temperature. The amplified RNA can then be utilized for mutation detection (Fahy et al., 1991, PCR Meth. Appl., 1:25-33). In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

As used herein, the term “sequence alignment” refers to a listing of multiple DNA or amino acid sequences and aligns them to highlight their similarities. The listings can be made using bioinformatics computer programs.

The term “sensitivity,” as used herein, is a measure which indicates the frequency of occurrence of a given genome sequence segment within a target genome.

The term “separation distance,” as used herein, refers to the intervening distance along a given genome sequence between two genome sequence segments chosen as primer hybridization sites. For example, a first genome sequence segment having genome coordinates 100-107 and a second genome sequence segment having genome coordinates of 200-207 have a separation distance of 92 nucleobases (genome coordinates 108 to 199).

The term “sepsis,” as used herein, refers to a serious medical condition resulting from the immune response to a severe infection. The related term “septicemia” is a sepsis of the bloodstream caused by bacteremia (the presence of bacteria in the bloodstream). The associated term “sepsis-causing organisms” refers to organisms that are frequently found in the blood when in the state of sepsis. Although the majority of sepsis-causing organisms are bacteria, fungi have also been identified in the blood of individuals with sepsis.

As used herein, the term “speciating primer pair” refers to a primer pair designed to produce a bioagent identifying amplicon with the diagnostic capability of identifying species members of a group of genera or a particular genus of bioagents. Primer pair number 2249 (SEQ ID NOs: 601:609), for example, is a speciating primer pair used to distinguish Staphylococcus aureus from other species of the genus Staphylococcus.

The terms “stopping criterion” and “stopping criteria” refer to a chosen minimal acceptable criterion or criteria of collections of genome sequence segments for inclusion in the set of selected genome sequence segments to which primers will be designed. Examples of stopping criteria include, but are not limited to values reflecting mean separation distance or maximum separation distance. These stopping criteria can be chosen to act as the final step in a method for primer design of primers useful with targeted genome amplification.

As used herein, a “sub-species characteristic” is a genetic characteristic that provides the means to distinguish two members of the same bioagent species. For example, one viral strain could be distinguished from another viral strain of the same species by possessing a genetic change (e.g., for example, a nucleotide deletion, addition or substitution) in one of the viral genes, such as the RNA-dependent RNA polymerase. Sub-species characteristics such as virulence genes and drug-are responsible for the phenotypic differences among the different strains of bacteria.

The term “target genome,” as used herein, refers to a genome of interest acting as the subject of analysis of the methods disclosed herein. For example, it is desirable to produce large quantities of a “target genome” while minimizing production of “background genomes.”

The terms “threshold criterion” and “threshold criteria,” as used herein refer to values reflecting characteristics of genome sequence segments at which selections of sub-sets of genome sequence segments are made. For example, sub-sets of genome sequence segments can be chosen using a threshold criterion of a selectivity ratio at or above the mean selectivity ratio.

As used herein, the term “targeted whole genome amplification primers” refers to primers collected in a set which are useful for selectively amplifying one or more target genome relative to one or more background genomes. Targeted whole genome amplification primers are designed according methods disclosed herein.

As used herein, the term “target genome sequence segment” refers to a portion of specified length of a genome which is desired to be selectively bound relative to one or more background genomes. Primers are selected to hybridize as selectively as possible to target genome sequence segments while minimizing hybridization to one or more background genomes.

The term “template” refers to a strand of nucleic acid on which a complementary copy is built from nucleoside triphosphates through the activity of a template-dependent nucleic acid polymerase. Within a duplex the template strand is, by convention, depicted and described as the “bottom” strand. Similarly, the non-template strand is often depicted and described as the “top” strand.

The term “triangulation genotyping analysis” refers to a method of genotyping a bioagent by measurement of molecular masses or base compositions of amplification products, corresponding to bioagent identifying amplicons, obtained by amplification of regions of more than one gene. In this sense, the term “triangulation” refers to a method of establishing the accuracy of information by comparing three or more types of independent points of view bearing on the same findings. Triangulation genotyping analysis carried out with a plurality of triangulation genotyping analysis primers yields a plurality of base compositions that then provide a pattern or “barcode” from which a species type can be assigned. The species type may represent a previously known sub-species or strain, or may be a previously unknown strain having a specific and previously unobserved base composition barcode indicating the existence of a previously unknown genotype.

As used herein, the term “triangulation genotyping analysis primer pair” is a primer pair designed to produce bioagent identifying amplicons for determining species types in a triangulation genotyping analysis.

The employment of more than one bioagent identifying amplicon for identification of a bioagent is herein referred to as “triangulation identification.” Triangulation identification is pursued by analyzing a plurality of bioagent identifying amplicons produced with different primer pairs. This process is used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected signatures from the B. anthracis genome would suggest a genetic engineering event.

As used herein, the term “unknown bioagent” may mean either: (i) a bioagent whose existence is known (such as the well known bacterial species Staphylococcus aureus for example) but which is not known to be in a sample to be analyzed, or (ii) a bioagent whose existence is not known (for example, the SARS coronavirus was unknown prior to April 2003). For example, if the method for identification of coronaviruses disclosed in commonly owned U.S. patent Ser. No. 10/829,826 (incorporated herein by reference in its entirety) was to be employed prior to April 2003 to identify the SARS coronavirus in a clinical sample, both meanings of “unknown” bioagent are applicable since the SARS coronavirus was unknown to science prior to April, 2003 and since it was not known what bioagent (in this case a coronavirus) was present in the sample. On the other hand, if the method of U.S. patent Ser. No. 10/829,826 was to be employed subsequent to April 2003 to identify the SARS coronavirus in a clinical sample, only the first meaning (i) of “unknown” bioagent would apply since the SARS coronavirus became known to science subsequent to April 2003 and since it was not known what bioagent was present in the sample.

The term “variable sequence” as used herein refers to differences in nucleic acid sequence between two nucleic acids. For example, the genes of two different bacterial species may vary in sequence by the presence of single base substitutions and/or deletions or insertions of one or more nucleotides. These two forms of the structural gene are said to vary in sequence from one another. As used herein, the term “viral nucleic acid” includes, but is not limited to, DNA, RNA, or DNA that has been obtained from viral RNA, such as, for example, by performing a reverse transcription reaction. Viral RNA can either be single-stranded (of positive or negative polarity) or double-stranded.

The term “virus” refers to obligate, ultramicroscopic, parasites that are incapable of autonomous replication (i.e., replication requires the use of the host cell's machinery). Viruses can survive outside of a host cell but cannot replicate.

The term “viremia” refers to a condition where viruses enter the bloodstream. It is similar to bacteremia, a condition where bacteria enter the bloodstream, and septicemia. Active viremia refers to the capability of the virus to replicate in blood. There are two types of viremia: primary viremia, which is the initial spread of virus in the blood; and secondary viremia, where the primary viremia has resulted in infection of additional tissues, in which the virus has replicated and once more entered the circulation.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product. As used herein, a “wobble base” is a variation in a codon found at the third nucleotide position of a DNA triplet. Variations in conserved regions of sequence are often found at the third nucleotide position due to redundancy in the amino acid code.

As used herein, the term “strand-displacing polymerase” refers to a polymerase capable of displacing a downstream nucleic acid (e.g., DNA) strand encountered during synthesis. Examples of strand-displacing polymerases include, but are not limited to, Phi29 base, Klenow polymerase, Bsu polymerase, Bst polymerase, Pyrophage® polymerase (Lucigen Corp.), Vent® polymerase (New England Biolabs), Deep Vent® polymerase (New England Biolabs), DyNAzyme™ EXT DNA polymerase (New England Biolabs), and 9° Nₘ DNA polymerase (New England Biolabs).

### 2. TARGETED AMPLIFICATION AND DETECTION METHOD

Provided herein is a method for targeted genome amplification. The target genome amplification may be of a whole genome or of part of a genome.

a. Target Genome

The target genome may be the genome of a target organism, which may be a bacterium or protozoan. The target genome may also a plurality of target genomes. The choice of target genomes is dictated by the objective of the analysis. For example, if the desired outcome of the targeted amplification process is to obtain nucleic acid representing the genome of a biowarfare organism such as Bacillus anthracis, which is suspected of being present in a soil sample at the scene of a biowarfare attack, one may choose to select the genome of Bacillus anthracis as the one and only target genome. If, on the other hand, the desired outcome of the targeted genome amplification process is to obtain nucleic acid representing a group of bacteria, such as, a group of potential biowarfare agents, more than one target genome may be selected such as, a group comprising any or all of the following bacteria: Bacillus anthracis, Francisella tularensis, Yersinia pestis, Brucella sp., Burkholderia mallei, Rickettsia prowazekii, and Escherichia coli 0157. Likewise, a different genome or group of genomes could be selected as the target genome(s) for other purposes. For example, a human genome or mitochondrial DNA may be the target over common genomes found in a soil sample or other sample environments where a crime may have taken place. Thus, the current methods and compositions can be applied and the human genome (target) selectively amplified over the background genomes. Other examples could include the genomes of group of viruses that cause respiratory illness, pathogens that cause sepsis, or a group of fungi known to contaminate households.

(1) Partial Target Genome

A partial target genome may also be selectively amplified over a background genome. The partial target genome may be contained in the target genome of a target organism. The partial genome may be a chromosome or a portion of a chromosome. The partial target genome may also comprise one or more target genes or sequences of interest. The target genes or sequences may be indicative of the target organism. The target genes or sequences may also be indicative of a group of organisms, such as a strain, a sub-species, a species, a genus, or any other phylogenetic group. For example, the target gene may encode a virulence factor.

b. Background Genome

The background genome may be selected based on the likelihood of the nucleic acid of certain organisms being present. The background genome may be nuclear DNA or organellar DNA, such as mitochondrial or chloroplast DNA. The background genome may be a plurality of nuclear or organellar genomes. For example, a soil sample which was handled by a human would be expected to contain nucleic acid representing the genomes of organisms including, but not limited to: Homo sapiens, Gallus gallus, Guillardia theta, Oryza sativa, Arabidopsis thaliana, Yarrowia lipolytica, Saccharomyces cerevisiae, Debaryomyces hansenii, Kluyveromyces lactis, Schizosaccharmyces pom, Aspergillus fumigatus, Cryptococcus neoformans, Encephalitozoon cuniculi, Eremothecium gossypii, Candida glabrata, Apis mellifera, Drosophila melanogaster, Tribolium castaneum, Anopheles gambiae, and Caenorhabditis elegans. Any or all of these genomes are appropriate to estimate as background genomes in the sample. The organisms actually in any particular sample will vary for each sample based upon the source and/or environment. Therefore, background genomes may be selected based upon the identities of organisms actually present in the sample. The composition of a sample can be determined using any of a number of techniques known to those ordinarily skilled in the art. The primers may be designed based upon actual identification of one or more background organisms in the sample, and based upon likelihood of any further one or more background organisms being in the sample.

c. Identification of Unique Genome Sequence Segments as Primer Hybridization Sites

Once the target and background genomes of a sample are determined, the next step is to identify genome sequence segments within the target genome which are useful as primer hybridization sites. The efficiency of a given targeted genome amplification is dependent on effective use of primers. To produce an amplification product representative of the target genome, the primer hybridization sites should have appropriate separation across the length of the genome. The mean separation distance between the primer hybridization sites may be about 1000, 900, 800, 700, 600, 500, 400, 300, 200, 100, 90, 80, 70, 60, or 50 nucleobases in length or less.

One with ordinary skill in the art will recognize that effective priming for targeted genome amplification depends upon several factors such as the fidelity and processivity of the polymerase enzyme used for primer extension. A longer mean separation distance between primer hybridization sites becomes more acceptable if the polymerase enzyme has high processivity. This indicates that the polymerase binds tightly to the nucleic acid template. This is a desirable characteristic for targeted genome amplification because it enables the polymerase to remain bound to the template nucleic acid and continue to extend the complementary nucleic acid strand being synthesized. Examples of polymerase enzymes having high processivity include, but are not limited to Phi29 polymerase and Taq polymerase. Protein engineering strategies have been used to produce high processivity polymerase enzymes, for example, by covalent linkage of a polymerase to a DNA-binding protein (Wang et al., Nucl. Acids Res., 2004, 32(3) 1197-1207). As polymerases with improved processivity become available, longer mean separation distances, even greatly exceeding 1000 nucleobases may be acceptable for targeted genome amplification.

d. Hybridization Sensitivity and Selectivity

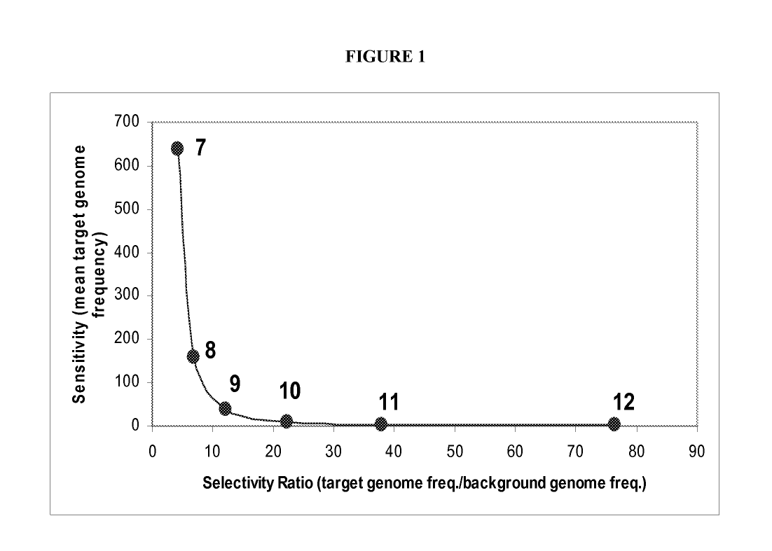

For the purpose of targeted genome amplification, the choice of length of the primer hybridization sites (genome sequence segments) and the lengths of the corresponding primers hybridizing thereto, preferably will balance two factors; (1) sensitivity, which indicates the frequency of binding of a given primer to the target genome, and (2) selectivity, which indicates the extent to which a given primer hybridizes to the target genome with greater frequency than it hybridizes to background genomes. Generally, longer primers tend toward greater selectivity and lesser sensitivity while the converse holds for shorter primers. The relationship between primer length, selectivity and sensitivity is graphically represented in FIG. 1. A primer may have a length of 5 to 100 nucleotides, and may be about 5 to about 13 nucleobases in length. The primer may have a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. Primer size affects the balance between selectivity of the primer and sensitivity of the primer. Optimal primer length is determined for each sample with this balance in mind. Choosing a plurality of primers having various lengths provide broad priming across the target genome sequence(s) while also providing preferential binding of the primers to the target genome sequence(s) relative to the background genome sequences.

e. Selection Threshold Criteria

A suitable sub-set of the total unique genome sequence segments may be determined in order to reduce the total number of primers in the targeted genome amplification set in order to reduce the costs and complexity of the primer set. The sub-set may also include genome sequence segments that can be used to select primers that amplify a partial target genome, rather than the whole genome. Determination of the suitable sub-set of unique genome sequence segments may entail choosing one or more threshold criteria which indicate a useful and practical cut-off point for sensitivity and/or selectivity of a given genome sequence segment. Examples of such criteria include, but are not limited to, a selected threshold frequency of occurrence (a frequency of occurrence threshold value), or a selected selectivity ratio (a selectivity ratio threshold value), such as a combined hit ratio.

The total unique genome sequence segments may be ranked according to the criteria. For example, the total unique genome sequence segments may be ranked according to frequency of occurrence with the #1 rank indicating the greatest frequency of occurrence and the lowest rank indicating the lowest frequency of occurrence. A threshold frequency of occurrence can then be chosen from the ranks The threshold frequency of occurrence serves as the dividing line between members of the sub-set chosen for further analysis and the members that will not be further analyzed.

The total unique genome sequence segments may also be ranked according to combined hit ratio. For example, the total unique genome sequence segments are ranked according to combined hit ratio with the #1 rank indicating the greatest combined hit ratio and the lowest rank indicating the lowest combined hit ratio. A threshold frequency of occurrence can then be set in order to choose unique genome segments. An iterative process of choosing unique genome sequence segments can be used to pick a subset of unique genome sequence segments that includes a predetermined number of unique genome sequence segments. The iterative process includes a first step, in which the unique genome sequence segment is selected having the highest combined hit ratio and a frequency of occurrence equal to or greater than the frequency threshold. In the second step, it is determined whether the unique genome sequence segment breaks up the largest remaining gap in target genome coverage. If yes, the unique genome sequence segment is added to the subset. If not, the unique genome sequence segment is discarded. The two steps are repeated until the predetermined number of unique genome sequence segments has been selected. This iterative process of choosing subsets of unique genome sequence segments can itself be repeated to select a plurality of subsets. Each time the iterative process is repeated, a higher frequency threshold can be set to select unique genome sequence segments. The first frequency threshold may be set to 0, but may also be set to a higher threshold as appropriate.

Given a defined maximum allowable distance between primer binding sites (a parameter) and a maximum allowable number of primers in a set that we wish to consider (another parameter), the method will generate a unique, repeatable set of primers. For example, to generate a set of primers that preferentially amplify a particular target genome over a background genome, the maximum allowable distance between primers may set to 1000 bp, and the primer set may consist of less than 200 primers. If the 1000 bp max distance constraint can be satisfied by 17 primers, then no other primers need be selected. If, on the other hand, the iterative process reaches 200 primers and the 1000 bp criterion is still satisfied, then the iterative process is started over with the criterion that a primer must hit the target genome N times where N is initially 0, and incremented each time the 1000 bp constraint cannot be satisfied, until the constraint is satisfied.

This algorithm produces a series of subsets of unique genome sequence segments, each with a different minimum frequency of occurrence within the target genome. The selection of sets of unique genome sequence segments introduces a trade-off. Subsets that have a higher combined hit ratio tend to also have a higher maximum separation distance between unique genome sequence segments. This is because unique genome sequence segments with a high combined hit ratio tend to be longer, such as 11 or 12 nucleotides, and tend to have a low frequency of occurrence in the background genome. These unique genome sequence segments also tend to have a lower frequency of occurrence in the target genome, but they are more selective for the target genome. A desirable subset of unique genome sequence segments balances this trade-off. The most important variables in the balancing process are the average combined hit ratio and the maximum separation distance between unique genome sequence segments. It may be preferable to choose a subset that includes unique genome sequence segments with a high average combined hit ratio and also a small maximum separation distance. A maximum separation distance between the unique genome sequence segments of about 500 nucleotides may be desirable. For partial target genome amplification, the maximum separation distance between unique genome sequences segments may be about 400, or 300, or 200, 100, 90, 80, 70, 60, or 50 nucleotides. If the average combined hit ratio of a subset is poor, however, it may be preferable to select a subset of unique genome sequence segments with a higher maximum separation distance.

In a non-limiting example, the mean “frequency of occurrence” can be calculated from the frequency of occurrence of the total genome sequence segments and this mean frequency of occurrence can be selected as a threshold criterion. The “frequency of occurrence” is defined in the “Definitions” section and also described in detail in Example 1. Genome sequence segments having a frequency of occurrence equal to or greater than the mean frequency of occurrence for all genome sequences being analyzed may be chosen as a sub-set for further analysis. The frequency of occurrence threshold criterion may also be chosen to be above the mean frequency of occurrence or below the mean frequency of occurrence. The sub-set may be chosen with a frequency of occurrence threshold criterion that defines the sub-set as consisting of 80%, 70%, 60% or 50% of the total unique genome sequence segments or any whole or fractional number therebetween.

A “selectivity ratio” may be chosen as the threshold criterion. The selectivity ratio is defined in the “Definitions” section and also described in detail in Example 1. All genome sequence segments having a selectivity ratio equal to or greater than the mean selectivity ratio may be chosen as a sub-set for further analysis. The selectivity ratio threshold criterion may also be chosen above the mean selectivity ratio or below the mean selectivity ratio. The sub-set may also be chosen with a selectivity ratio threshold criterion that defines the sub-set as consisting of 80%, 70%, 60% or 50% of the total unique genome sequence segments or any whole or fractional number therebetween.

Choosing the target genome sequence segments that are useful as primer hybridization sites may be facilitated by the identification of most, if not all, of the unique genome sequence segments with lengths of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleobases from which the primer hybridization sites will be chosen. Identification of unique sequence segments within genome sequences itself is a procedure that is well known to those with ordinary skill in bioinformatics. Furthermore, determination of the frequency of occurrence of a given genome sequence segment can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). One with ordinary skill will recognize that improvements in polymerase processivity through, for example, protein engineering, discovery of new polymerases or improvements in amplification reagents and methods will allow for a shift in the balance between selectivity and sensitivity toward selectivity because a polymerase with improved processivity can synthesize longer stretches of primer extension products without the need for high frequency of occurrence of shorter genome sequence segments acting as hybridization sites for shorter primers. Thus, primer lengths above 13 nucleobases are also practical for use in targeted genome amplification.

Example 1 provides a demonstration of identification of unique genome sequence segments within a target genome, determination of the frequencies of occurrence of the genome sequence segments within the target genome sequence and determination of the frequencies of occurrence of the genome sequence segments within the background genome sequences. The example further describes calculation and ranking of selectivity ratios using the frequencies of occurrence of genome sequence segments within the target genomes and within the background genomes. In brief, selectivity ratios provide a description of the selectivity of a given genome sequence segment towards the target genome(s) with respect to the background genomes. A selectivity ratio is calculated for a given genome sequence segment simply by dividing the frequency of occurrence of the genome sequence segment within the target genome(s) by the frequency of occurrence of the genome sequence segment in the background genomes. A high selectivity ratio for a given genome sequence segment is favorable because it indicates that a primer designed to hybridize to the genome sequence segment will hybridize to the target genome(s) more frequently than it will hybridize to the background genomes, thus, accomplishing one objective for selective priming of the target genome. Selectivity ratios can be calculated either for a single target genome or for a plurality of target genomes. It is advantageous to consider the frequency of occurrence of all genome sequence segments in all of the chosen background genome segments to obtain useful selectivity ratios but, depending on the objective of the targeted genome amplification, it is not typically necessary to consider all possible target genomes in calculation of selectivity ratios. For example, in a simplified system consisting of two target genomes (target genome A and target genome B) and three background genomes (background genomes C, D and E), the selectivity ratio for genome sequence segment X which occurs once (frequency of occurrence=1) in A, B, C, D and E, the target genome A selectivity ratio would be calculated as follows:

1(A)/(1(C)+1(D)+1(E))=0.333

In contrast, the total target genome (A+B) selectivity ratio would be calculated as follows:

1(A)+1(B)/(1(C)+1(D)+1(E)=0.667

The selectivity ratio may also be a hit ratio or combined hit ratio as described herein. The methods for selecting primers for targeted genome amplification and the algorithms disclosed herein may be performed using a computer-based method. The computer-based method may comprise an input for inputting the genome sequences of interest and parameters for performing the primer selection methods and algorithms described herein into the memory of a computer, and an output, that displays the results of the primer selection methods and algorithms. The computer-based method may comprise a database of genome sequences, and an algorithm for identifying sequence similarity, such as a BLAST algorithm. The computer-based method may comprise entry or selection of the genome sequences and parameters for performing the primer selection methods and algorithms, and execution of the primer selection methods and algorithms. The output of the primer selection methods and algorithms may be a file, such as a table, and the file may be stored in the memory of the computer.

### 3. TARGETED GENOME CAPTURE PROBES

A primer for targeted genome amplification as described herein may be used as a capture probe. A primer set or portion thereof as described herein may also be used as a capture probe. A capture probe may be used to detect a target genome or a target partial genome. The capture probe may be immobilized according to a Synchronous Coefficient of Drag Alteration (SCODA) method as described in International Application No. PCT/US10/26550, the contents of which are incorporated herein by reference. The capture probe may allow for selective concentration of the target genome from the background genome. The target genome may then be subjected to targeted genome amplification by using a primer or plurality of primers having the same sequence as the capture probe or probes. The probe may also be a real-time probe, a scorpion probe, a hybridization probe, a 5′-nuclease probe, a molecular beacon probe, and a FISH probe. The probe may also be attached to a microarray or HPLC.

### 4. TARGETED GENOME AMPLIFICATION PRIMER KITS

Also provided herein is a kit that includes targeted genome amplification primers designed according to the methods disclosed herein. The kit may comprise primers designed for general targeted genome amplification of bacteria from one or more collections of background genomes. For example, a targeted genome amplification kit for identification of bacteria in soil may have primers selected based on the genomes of typical background organisms found in soil. In another example, a targeted genome amplification kit for genotyping of viruses causing respiratory illness might be assembled with primers selected based on the target genomes of the respiratory pathogens and background genomes including the human genome and the genomes of commensal organisms found in human mucus, or other fluids. In another example, a targeted genome amplification kit for genotyping of sepsis-causing bacteria might be assembled with primers selected based on the target genomes of the sepsis-causing bacteria and background genomes including the human genome. Since human blood generally does not contain significant quantities of bacteria under non-sepsis conditions, bacterial genomes generally not be included in the primer selection process for this kit.

The kit may comprise a sufficient quantity of a polymerase enzyme having high processivity. The high processivity polymerase may be Phi29 polymerase or Taq polymerase. The high processivity polymerase may be a genetically engineered polymerase whose processivity is increased relative to the native polymerase from which it was constructed. The kit may further comprise deoxynucleotide triphosphates, buffers, buffer additives such as magnesium salts, trehalose and betaine at concentrations optimized for targeted genome amplification. The kit may also further comprise instructions for carrying out targeted genome amplification reactions.

### 5. PROGRAMMING, COMPUTER READABLE MEDIA AND COMPUTER SYSTEMS

Provided herein is computer programming written on computer readable media for performing the methods set forth herein. While the subject programming finds use in a variety of settings, it is most commonly used in a computer system comprising a processor, a memory, an input, and an output that are coupled to each other.

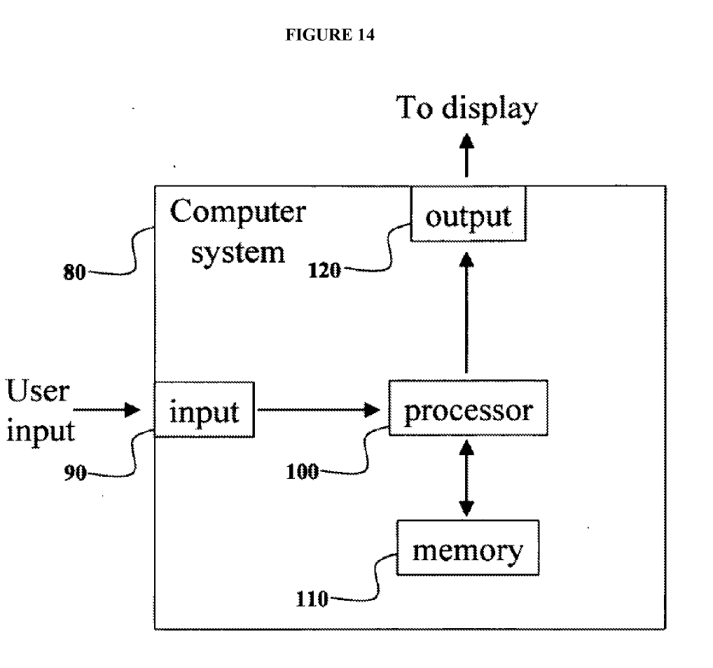

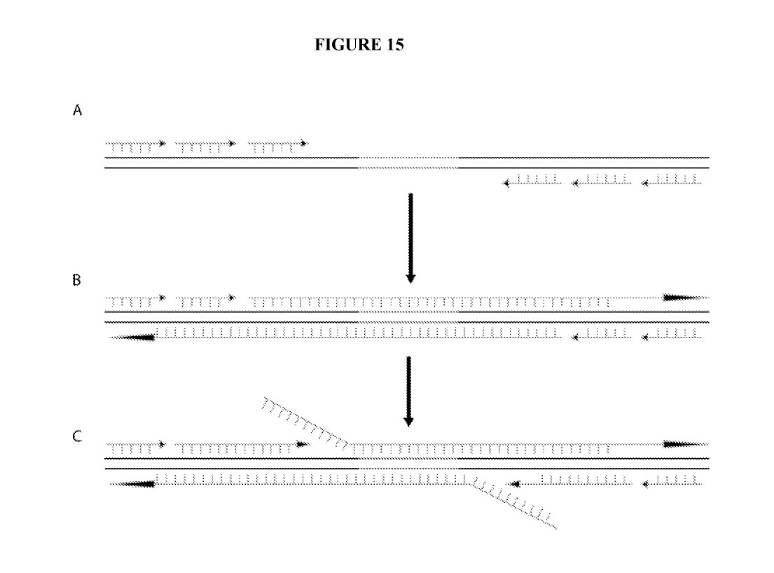

FIG. 14 is a simplified block diagram of computer system 80. Computer system 80 may include at least one processor 100 that communicates with a peripheral device. The peripheral device may include a memory 110, a user interface input device 90, user interface output device 120 (e.g. a monitor). The input and output devices may allow user interaction with computer system 80. The user may be a human user, a device, or another computer.

The user interface input device 90 may include a keyboard, a pointing device such as a mouse, trackball, touchpad, or graphics tablet, a scanner, a touchscreen incorporated into the display, an audio input device such as a voice recognition system, microphone, or other types of input device. The term “input device” may include any possible type of devices and ways to input information into computer system 80.

User interface output device 120 may include a display subsystem, a printer, a fax machine, or non-visual display such as an audio output device. The display subsystem may be a cathode ray tube (CRT), a flat-panel device such as a liquid crystal display (LCD), or a projection device. The display subsystem may also provide non-visual display such as via audio output devices. The term “output device” may include any possible types of devices and ways to output information from computer system 80 to a human or to another machine or computer system.

Memory 110 stores the basic programming and data constructs that provide the functionality of the various systems described herein. For example, an algorithm for performing a method set forth above may be stored in memory 110 as a software module. The software module may be executed by processor 100. In a distributed environment, the software module may be stored on a plurality of computer systems and executed by processors of the plurality of computer systems. Memory 110 also provides a repository for storing the various databases storing information described herein.

Memory 110 may include a number of memories including a main random access memory (RAM) for storage of instructions and data during program execution and a read only memory (ROM) in which fixed instructions are stored. A file storage subsystem may provide persistent (non-volatile) storage for program and data files, and may include a computer readable media, e.g., a hard disk drive, a floppy disk drive along with associated removable media, a Compact Digital Read Only Memory (CD-ROM) drive, an optical drive, removable media cartridges, and other like storage media. One or more of the drives may be located at remote locations on other connected computers at another site on a communication network.

Computer system 80 may be a personal computer, a portable computer, a workstation, a computer terminal, a network computer, a television, a mainframe, or any other data processing system. Due to the ever-changing nature of computers and networks, the description of computer system 80 depicted in FIG. 14 is intended only as a specific example for purposes of illustrating a common embodiment of the present invention. Many other configurations of a computer system are possible having more or less components than the computer system depicted in FIG. 14.

### 6. BIOAGENT IDENTIFYING AMPLICONS

Disclosed herein are methods for detection and identification of unknown bioagents using bioagent identifying amplicons. Primers as described above are further selected from the pool of primers to hybridize to conserved sequence regions of nucleic acids derived from a bioagent, and which bracket variable sequence regions to yield a bioagent identifying amplicon, which can be amplified and which is amenable to molecular mass determination. The molecular mass then provides a means to uniquely identify the bioagent without a requirement for prior knowledge of the possible identity of the bioagent. The molecular mass or corresponding base composition signature of the amplification product is then matched against a database of molecular masses or base composition signatures. A match is obtained when an experimentally-determined molecular mass or base composition of an analyzed amplification product is compared with known molecular masses or base compositions of known bioagent identifying amplicons and the experimentally determined molecular mass or base composition is the same as the molecular mass or base composition of one of the known bioagent identifying amplicons. Alternatively, the experimentally-determined molecular mass or base composition may be within experimental error of the molecular mass or base composition of a known bioagent identifying amplicon and still be classified as a match. In some cases, the match may also be classified using a probability of match model such as the models described in U.S. Ser. No. 11/073,362, which is commonly owned and incorporated herein by reference in entirety. Furthermore, the method can be applied to rapid parallel multiplex analyses, the results of which can be employed in a triangulation identification strategy. The present method provides rapid throughput and does not require nucleic acid sequencing of the amplified target sequence for bioagent detection and identification.

Despite enormous biological diversity, all forms of life on earth share sets of essential, common features in their genomes. Since genetic data provide the underlying basis for identification of bioagents by the methods disclosed herein, it is necessary to select segments of nucleic acids which ideally provide enough variability to distinguish each individual bioagent and whose molecular mass is amenable to molecular mass determination.

Unlike bacterial genomes, which exhibit conservation of numerous genes (i.e. housekeeping genes) across all organisms, viruses do not share a gene that is essential and conserved among all virus families. Therefore, viral identification is achieved within smaller groups of related viruses, such as members of a particular virus family or genus. For example, RNA-dependent RNA polymerase is present in all single-stranded RNA viruses and can be used for broad priming as well as resolution within the virus family.

At least one bacterial nucleic acid segment may be amplified in the process of identifying the bacterial bioagent. Thus, the nucleic acid segments that can be amplified by the primers disclosed herein and that provide enough variability to distinguish each individual bioagent and whose molecular masses are amenable to molecular mass determination are herein described as bioagent identifying amplicons.

Bioagent identifying amplicons may comprise from about 27 to about 200 nucleobases (i.e. from about 39 to about 200 linked nucleosides), although both longer and short regions may be used. One of ordinary skill in the art will appreciate that these embodiments include compounds of 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 nucleobases in length, or any range therewithin.

It is the combination of the portions of the bioagent nucleic acid segment to which the primers hybridize (hybridization sites) and the variable region between the primer hybridization sites that comprises the bioagent identifying amplicon. Thus, it can be said that a given bioagent identifying amplicon is “defined by” a given pair of primers.

Bioagent identifying amplicons amenable to molecular mass determination which are produced by the primers described herein may be either of a length, size or mass compatible with the particular mode of molecular mass determination or compatible with a means of providing a predictable fragmentation pattern in order to obtain predictable fragments of a length compatible with the particular mode of molecular mass determination. Such means of providing a predictable fragmentation pattern of an amplification product include, but are not limited to, cleavage with chemical reagents, restriction enzymes or cleavage primers, for example. Thus, bioagent identifying amplicons may be larger than 200 nucleobases and may be amenable to molecular mass determination following restriction digestion. Methods of using restriction enzymes and cleavage primers are well known to those with ordinary skill in the art.

Amplification products corresponding to bioagent identifying amplicons may be obtained using the polymerase chain reaction (PCR) that is a routine method to those with ordinary skill in the molecular biology arts. Other amplification methods may be used such as ligase chain reaction (LCR), low-stringency single primer PCR, and multiple strand displacement amplification (MDA). These methods are also known to those with ordinary skill.

### 7. PRIMER PAIRS THAT DEFINE BIOAGENT IDENTIFYING AMPLICONS

The primers may be designed to bind to conserved sequence regions of a bioagent identifying amplicon that flank an intervening variable region and yield amplification products which provide variability sufficient to distinguish each individual bioagent, and which are amenable to molecular mass analysis. The highly conserved sequence regions may exhibit between about 80-100%, or between about 90-100%, or between about 95-100% identity, or between about 99-100% identity. The molecular mass of a given amplification product provides a means of identifying the bioagent from which it was obtained, due to the variability of the variable region. Thus, design of the primers involves selection of a variable region with sufficient variability to resolve the identity of a given bioagent. Bioagent identifying amplicons may be specific to the identity of the bioagent.

Identification of bioagents may be accomplished at different levels using primers suited to resolution of each individual level of identification. Broad range survey primers are designed with the objective of identifying a bioagent as a member of a particular division (e.g., an order, family, genus or other such grouping of bioagents above the species level of bioagents). Broad range survey intelligent primers may be capable of identification of bioagents at the species or sub-species level. Examples of broad range survey primers include, but are not limited to: primer pair numbers: 346 (SEQ ID NOs: 594:602), and 348 (SEQ ID NOs: 595:603) which target DNA encoding 16S rRNA, and primer pair number 349 (SEQ ID NOs: 596:604) which targets DNA encoding 23S rRNA. Additional broad range survey primer pairs are disclosed in U.S. Ser. No. 11/409,535 which is incorporated herein by reference in entirety.

Drill-down primers may be designed with the objective of identifying a bioagent at the sub-species level (including strains, subtypes, variants and isolates) based on sub-species characteristics which may, for example, include single nucleotide polymorphisms (SNPs), variable number tandem repeats (VNTRs), deletions, drug resistance mutations or any other modification of a nucleic acid sequence of a bioagent relative to other members of a species having different sub-species characteristics. Drill-down intelligent primers are not always required for identification at the sub-species level because broad range survey intelligent primers may, in some cases provide sufficient identification resolution to accomplishing this identification objective. Examples of drill-down primers are disclosed in U.S. patent application Ser. No. 11/409,535 which is incorporated herein by reference in entirety.

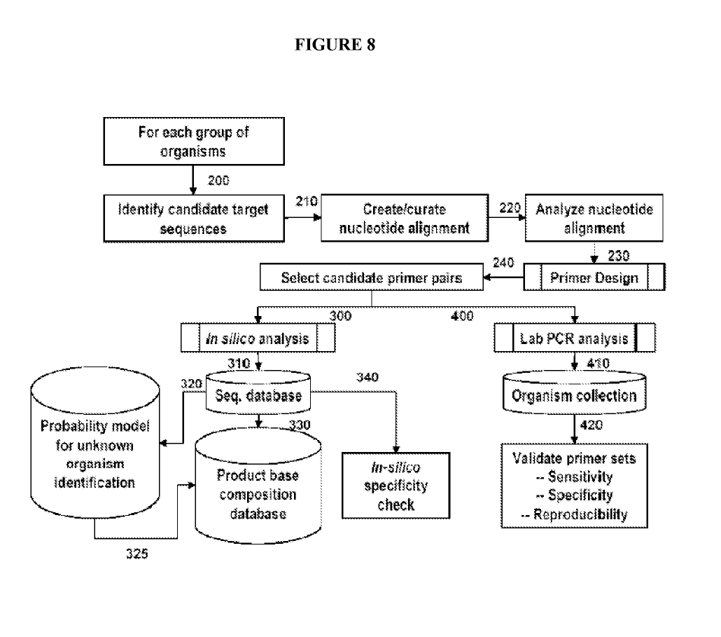

A representative process flow diagram used for primer selection and validation process is outlined in FIG. 8. For each group of organisms, candidate target sequences are identified (200) from which nucleotide alignments are created (210) and analyzed (220). Primers are then designed by selecting appropriate priming regions (230) to facilitate the selection of candidate primer pairs (240). The primer pairs are then subjected to in silico analysis by electronic PCR (ePCR) (300) wherein bioagent identifying amplicons are obtained from sequence databases such as GenBank or other sequence collections (310) and checked for specificity in silico (320). Bioagent identifying amplicons obtained from GenBank sequences (310) can also be analyzed by a probability model which predicts the capability of a given amplicon to identify unknown bioagents such that the base compositions of amplicons with favorable probability scores are then stored in a base composition database (325). Alternatively, base compositions of the bioagent identifying amplicons obtained from the primers and GenBank sequences can be directly entered into the base composition database (330). Candidate primer pairs (240) are validated by testing their ability to hybridize to target nucleic acid by an in vitro amplification by a method such as PCR analysis (400) of nucleic acid from a collection of organisms (410). Amplification products thus obtained are analyzed by gel electrophoresis or by mass spectrometry to confirm the sensitivity, specificity and reproducibility of the primers used to obtain the amplification products (420).

Many important pathogens, including the organisms of greatest concern as biowarfare agents, have been completely sequenced. This effort has greatly facilitated the design of primers for the detection of unknown bioagents. The combination of broad-range priming with division-wide and drill-down priming has been used very successfully in several applications of the technology, including environmental surveillance for biowarfare threat agents and clinical sample analysis for medically important pathogens.

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. However, it should be noted that “synthesis” of primers does not equate with “design” of primers. The primers disclosed herein have been designed by the methods disclosed herein and then synthesized by the known methods. Primers may be employed as compositions for use in methods for identification of bacterial bioagents as follows: a primer pair composition is contacted with nucleic acid (such as, for example, bacterial DNA or DNA reverse transcribed from the rRNA) of an unknown bacterial bioagent. The nucleic acid is then amplified by a nucleic acid amplification technique, such as PCR for example, to obtain an amplification product that represents a bioagent identifying amplicon. The molecular mass of each strand of the double-stranded amplification product is determined by a molecular mass measurement technique such as mass spectrometry for example, wherein the two strands of the double-stranded amplification product are separated during the ionization process. The mass spectrometry may be electrospray Fourier transform ion cyclotron resonance mass spectrometry (ESI-FTICR-MS) or electrospray time of flight mass spectrometry (ESI-TOF-MS). A list of possible base compositions can be generated for the molecular mass value obtained for each strand and the choice of the correct base composition from the list is facilitated by matching the base composition of one strand with a complementary base composition of the other strand. The molecular mass or base composition thus determined is then compared with a database of molecular masses or base compositions of analogous bioagent identifying amplicons for known bacterial bioagents. A match between the molecular mass or base composition of the amplification product and the molecular mass or base composition of an analogous bioagent identifying amplicon for a known viral bioagent indicates the identity of the unknown bacterial bioagent. The method may be repeated using one or more different primer pairs to resolve possible ambiguities in the identification process or to improve the confidence level for the identification assignment.

A bioagent identifying amplicon may be produced using only a single primer (either the forward or reverse primer of any given primer pair), provided an appropriate amplification method is chosen, such as, for example, low stringency single primer PCR (LSSP-PCR). Adaptation of this amplification method in order to produce bioagent identifying amplicons can be accomplished by one with ordinary skill in the art without undue experimentation.

The molecular mass or base composition of a bacterial bioagent identifying amplicon defined by a broad range survey primer pair may not provide enough resolution to unambiguously identify a bacterial bioagent at or below the species level. These cases benefit from further analysis of one or more bacterial bioagent identifying amplicons generated from at least one additional broad range survey primer pair or from at least one additional division-wide primer pair. The employment of more than one bioagent identifying amplicon for identification of a bioagent is herein referred to as triangulation identification.

The oligonucleotide primers may be division-wide primers which hybridize to nucleic acid encoding genes of species within a genus of bacteria. The oligonucleotide primers may be drill-down primers which enable the identification of sub-species characteristics. Drill down primers provide the functionality of producing bioagent identifying amplicons for drill-down analyses such as strain typing when contacted with nucleic acid under amplification conditions. Identification of such sub-species characteristics is often critical for determining proper clinical treatment of viral infections. In some embodiments, sub-species characteristics are identified using only broad range survey primers and division-wide and drill-down primers are not used. The primers used for amplification may hybridize to and amplify genomic DNA, and DNA of bacterial plasmids.

Various computer software programs may be used to aid in design of primers for amplification reactions such as Primer Premier 5 (Premier Biosoft, Palo Alto, Calif.) or OLIGO Primer Analysis Software (Molecular Biology Insights, Cascade, Colo.). These programs allow the user to input desired hybridization conditions such as melting temperature of a primer-template duplex for example. An in silico PCR search algorithm, such as (ePCR) may be used to analyze primer specificity across a plurality of template sequences which can be readily obtained from public sequence databases such as GenBank for example. An existing RNA structure search algorithm (Macke et al., Nucl. Acids Res., 2001, 29, 4724-4735, the contents of which are incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This also provides information on primer specificity of the selected primer pairs. In some embodiments, the hybridization conditions applied to the algorithm can limit the results of primer specificity obtained from the algorithm. In some embodiments, the melting temperature threshold for the primer template duplex is specified to be 35° C. or a higher temperature. In some embodiments the number of acceptable mismatches is specified to be seven mismatches or less. In some embodiments, the buffer components and concentrations and primer concentrations may be specified and incorporated into the algorithm, for example, an appropriate primer concentration is about 250 nM and appropriate buffer components are 50 mM sodium or potassium and 1.5 mM Mg²⁺.

One with ordinary skill in the art of design of amplification primers will recognize that a given primer need not hybridize with 100% complementarity in order to effectively prime the synthesis of a complementary nucleic acid strand in an amplification reaction. Moreover, a primer may hybridize over one or more segments such that intervening or adjacent segments are not involved in the hybridization event. (e.g., for example, a loop structure or a hairpin structure). The primers may comprise at least 70%, at least 75%, at least 80%, at least 85%, at least 90%, at least 95% or at least 99% sequence identity with any of the primers listed in Table 2 of U.S. Ser. No. 11/409,535, the contents of which are incorporated herein by reference in entirety. Thus, in some embodiments, an extent of variation of 70% to 100%, or any range therewithin, of the sequence identity is possible relative to the specific primer sequences disclosed herein. Determination of sequence identity is described in the following example: a primer 20 nucleobases in length which is identical to another 20 nucleobase primer having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer.

Percent homology, sequence identity or complementarity, can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). Complementarity of primers with respect to the conserved priming regions of viral nucleic acid may be between about 70% and about 75% 80%. Homology, sequence identity or complementarity, may be between about 75% and about 80%. Homology, sequence identity or complementarity, may also be at least 85%, at least 90%, at least 92%, at least 94%, at least 95%, at least 96%, at least 97%, at least 98%, at least 99% or is 100%.

The primers described herein may comprise at least 70%, at least 75%, at least 80%, at least 85%, at least 90%, at least 92%, at least 94%, at least 95%, at least 96%, at least 98%, or at least 99%, or 100% (or any range therewithin) sequence identity with the primer sequences specifically disclosed herein.

One with ordinary skill is able to calculate percent sequence identity or percent sequence homology and able to determine, without undue experimentation, the effects of variation of primer sequence identity on the function of the primer in its role in priming synthesis of a complementary strand of nucleic acid for production of an amplification product of a corresponding bioagent identifying amplicon.

The primers may be at least 13 nucleobases in length. The primers may also be less than 36 nucleobases in length. The oligonucleotide primers may be 13, 14, 15, 16, 17, 18, 19, 20, 21, 22, 23, 24, 25, 26, 27, 28, 29, 30, 31, 32, 33, 34 or 35 nucleobases in length, or any range therewithin. The methods disclosed herein contemplate use of both longer and shorter primers. Furthermore, the primers may also be linked to one or more other desired moieties, including, but not limited to, affinity groups, ligands, regions of nucleic acid that are not complementary to the nucleic acid to be amplified, labels, etc. Primers may also form hairpin structures. For example, hairpin primers may be used to amplify short target nucleic acid molecules. The presence of the hairpin may stabilize the amplification complex (see e.g., TAQMAN MicroRNA Assays, Applied Biosystems, Foster City, Calif.).

Any oligonucleotide primer pair may have one or both primers with less then 70% sequence homology with a corresponding member of any of the primer pairs of Table 2 of U.S. Ser. No. 11/409,535, if the primer pair has the capability of producing an amplification product corresponding to a bioagent identifying amplicon. Any oligonucleotide primer pair may have one or both primers with a length greater than 35 nucleobases if the primer pair has the capability of producing an amplification product corresponding to a bioagent identifying amplicon. The function of a given primer may be substituted by a combination of two or more primers segments that hybridize adjacent to each other or that are linked by a nucleic acid loop structure or linker which allows a polymerase to extend the two or more primers in an amplification reaction.

The primer pairs used for obtaining bioagent identifying amplicons may be the primer pairs of Table 2 of U.S. Ser. No. 11/409,535. Other combinations of primer pairs may be possible by combining certain members of the forward primers with certain members of the reverse primers. An example can be seen in Table 2 of U.S. Ser. No. 11/409,535, for two primer pair combinations of forward primer 16S_EC_{—}789_{—}810 F with the reverse primers 16S_EC_{—}880_{—}894 R or 16S_EC_{—}882_{—}899 R. Arriving at a favorable alternate combination of primers in a primer pair depends upon the properties of the primer pair, most notably the size of the bioagent identifying amplicon that is defined by the primer pair, which preferably is between about 39 to about 200 nucleobases in length. Alternatively, a bioagent identifying amplicon longer than 200 nucleobases in length could be cleaved into smaller segments by cleavage reagents such as chemical reagents, or restriction enzymes, for example.

The primers may be configured to amplify nucleic acid of a bioagent to produce amplification products that can be measured by mass spectrometry and from whose molecular masses candidate base compositions can be readily calculated.

Any given primer may comprise a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated adenosine residues as a result of the non-specific enzyme activity of Taq polymerase (Magnuson et al., Biotechniques, 1996, 21, 700-709), an occurrence which may lead to ambiguous results arising from molecular mass analysis. Primers may contain one or more universal bases. Because any variation (due to codon wobble in the 3rd position) in the conserved regions among species is likely to occur in the third position of a DNA (or RNA) triplet, oligonucleotide primers can be designed such that the nucleotide corresponding to this position is a base which can bind to more than one nucleotide, referred to herein as a “universal nucleobase.” For example, under this “wobble” pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides, 1995, 14, 1001-1003), the degenerate nucleotides dP or dK (Hill et al.), an acyclic nucleoside analog containing 5-nitroindazole (Van Aerschot et al., Nucleosides and Nucleotides, 1995, 14, 1053-1056) or the purine analog 1-(2-deoxy-.beta.-D-ribofuranosyl)-imidazole-4-carboxamide (Sala et al., Nucl. Acids Res., 1996, 24, 3302-3306).

To compensate for the somewhat weaker binding by the wobble base, the oligonucleotide primers may be designed such that the first and second positions of each triplet are occupied by nucleotide analogs that bind with greater affinity than the unmodified nucleotide. Examples of these analogs include, but are not limited to, 2,6-diaminopurine which binds to thymine, 5-propynyluracil (also known as propynylated thymine) which binds to adenine and 5-propynylcytosine and phenoxazines, including G-clamp, which binds to G. Propynylated pyrimidines are described in U.S. Pat. Nos. 5,645,985, 5,830,653 and 5,484,908, the contents of all of which are incorporated herein by reference in their entirety. Propynylated primers are described in U.S. Pre-Grant Publication No. 2003-0170682, which is also commonly owned and the contents of which are incorporated herein by reference in their entirety. Phenoxazines are described in U.S. Pat. Nos. 5,502,177, 5,763,588, and 6,005,096, the contents of all of which are incorporated herein by reference in their entirety. G-clamps are described in U.S. Pat. Nos. 6,007,992 and 6,028,183, the contents of which are incorporated herein by reference in their entirety.

Primer hybridization may be enhanced using primers containing 5-propynyl deoxycytidine and deoxythymidine nucleotides. These modified primers offer increased affinity and base pairing selectivity.

Non-template primer tags may be used to increase the melting temperature (Tm) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

Propynylated tags may be used in a manner similar to that of the non-template tag, wherein two or more 5-propynylcytidine or 5-propynyluridine residues replace template matching residues on a primer. A primer may contain a modified internucleoside linkage such as a phosphorothioate linkage, for example.

The primers may contain mass-modifying tags. Reducing the total number of possible base compositions of a nucleic acid of specific molecular weight provides a means of avoiding a persistent source of ambiguity in determination of base composition of amplification products. Addition of mass-modifying tags to certain nucleobases of a given primer will result in simplification of de novo determination of base composition of a given bioagent identifying amplicon from its molecular mass.

The mass modified nucleobase may comprise one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. The mass-modified nucleobase may comprise 15N or 13C or both 15N and 13C.

Multiplex amplification may be performed where multiple bioagent identifying amplicons are amplified with a plurality of primer pairs. The advantages of multiplexing are that fewer reaction containers (for example, wells of a 96- or 384-well plate) are needed for each molecular mass measurement, providing time, resource and cost savings because additional bioagent identification data can be obtained within a single analysis. Multiplex amplification methods are well known to those with ordinary skill and can be developed without undue experimentation. However, one useful and non-obvious step in selecting a plurality candidate bioagent identifying amplicons for multiplex amplification may be to ensure that each strand of each amplification product will be sufficiently different in molecular mass that mass spectral signals will not overlap and lead to ambiguous analysis results. In some embodiments, a 10 Da difference in mass of two strands of one or more amplification products is sufficient to avoid overlap of mass spectral peaks.

As an alternative to multiplex amplification, single amplification reactions may be pooled before analysis by mass spectrometry. In these embodiments, as for multiplex amplification embodiments, it is useful to select a plurality of candidate bioagent identifying amplicons to ensure that each strand of each amplification product will be sufficiently different in molecular mass that mass spectral signals will not overlap and lead to ambiguous analysis results.

### 8. DETERMINATION OF MOLECULAR MASS OF BIOAGENT IDENTIFYING AMPLICONS

The molecular mass of a given bioagent identifying amplicon may be determined by mass spectrometry. Mass spectrometry has several advantages, not the least of which is high bandwidth characterized by the ability to separate (and isolate) many molecular peaks across a broad range of mass to charge ratio (m/z). Thus mass spectrometry is intrinsically a parallel detection scheme without the need for radioactive or fluorescent labels, since every amplification product is identified by its molecular mass. The current state of the art in mass spectrometry is such that less than femtomole quantities of material can be readily analyzed to afford information about the molecular contents of the sample. An accurate assessment of the molecular mass of the material can be quickly obtained, irrespective of whether the molecular weight of the sample is several hundred, or in excess of one hundred thousand atomic mass units (amu) or Daltons. Intact molecular ions may be generated from amplification products using one of a variety of ionization techniques to convert the sample to gas phase. These ionization methods include, but are not limited to, electrospray ionization (ES), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation. The mass detectors used in the methods described herein include, but are not limited to, Fourier transform ion cyclotron resonance mass spectrometry (FT-ICR-MS), time of flight (TOF), ion trap, quadrupole, magnetic sector, Q-TOF, and triple quadrupole.

### 9. BASE COMPOSITIONS OF BIOAGENT IDENTIFYING AMPLICONS

Although the molecular mass of amplification products obtained using intelligent primers provides a means for identification of bioagents, conversion of molecular mass data to a base composition signature is useful for certain analyses. As used herein, “base composition” is the exact number of each nucleobase (A, T, C and G) determined from the molecular mass of a bioagent identifying amplicon. In some embodiments, a base composition provides an index of a specific organism. Base compositions can be calculated from known sequences of known bioagent identifying amplicons and can be experimentally determined by measuring the molecular mass of a given bioagent identifying amplicon, followed by determination of all possible base compositions which are consistent with the measured molecular mass within acceptable experimental error. The following example illustrates determination of base composition from an experimentally obtained molecular mass of a 46-mer amplification product originating at position 1337 of the 16S rRNA of Bacillus anthracis. The forward and reverse strands of the amplification product have measured molecular masses of 14208 and 14079 Da, respectively. The possible base compositions derived from the molecular masses of the forward and reverse strands for the Bacillus anthracis products are listed in Table 1.

TABLE 1
Possible Base Compositions for B. anthracis 46mer Amplification Product

| Calc. Mass | Mass Error | Base Composition | Calc. Mass | Mass Error | Base Composition |
|---|---|---|---|---|---|
| Forward Strand | Forward Strand | of Forward Strand | Reverse Strand | Reverse Strand | of Reverse Strand |
| 14208.2935 | 0.079520 | A1 G17 C10 T18 | 14079.2624 | 0.080600 | A0 G14 C13 T19 |
| 14208.3160 | 0.056980 | A1 G20 C15 T10 | 14079.2849 | 0.058060 | A0 G17 C18 T11 |
| 14208.3386 | 0.034440 | A1 G23 C20 T2 | 14079.3075 | 0.035520 | A0 G20 C23 T3 |
| 14208.3074 | 0.065560 | A6 G11 C3 T26 | 14079.2538 | 0.089180 | A5 G5 C1 T35 |
| 14208.3300 | 0.043020 | A6 G14 C8 T18 | 14079.2764 | 0.066640 | A5 G8 C6 T27 |
| 14208.3525 | 0.020480 | A6 G17 C13 T10 | 14079.2989 | 0.044100 | A5 G11 C11 T19 |
| 14208.3751 | 0.002060 | A6 G20 C18 T2 | 14079.3214 | 0.021560 | A5 G14 C16 T11 |
| 14208.3439 | 0.029060 | A11 G8 C1 T26 | 14079.3440 | 0.000980 | A5 G17 C21 T3 |
| 14208.3665 | 0.006520 | A11 G11 C6 T18 | 14079.3129 | 0.030140 | A10 G5 C4 T27 |
| 14208.3890 | 0.016020 | A11 G14 C11 T10 | 14079.3354 | 0.007600 | A10 G8 C9 T19 |
| 14208.4116 | 0.038560 | A11 G17 C16 T2 | 14079.3579 | 0.014940 | A10 G11 C14 T11 |
| 14208.4030 | 0.029980 | A16 G8 C4 T18 | 14079.3805 | 0.037480 | A10 G14 C19 T3 |
| 14208.4255 | 0.052520 | A16 G11 C9 T10 | 14079.3494 | 0.006360 | A15 G2 C2 T27 |
| 14208.4481 | 0.075060 | A16 G14 C14 T2 | 14079.3719 | 0.028900 | A15 G5 C7 T19 |
| 14208.4395 | 0.066480 | A21 G5 C2 T18 | 14079.3944 | 0.051440 | A15 G8 C12 T11 |
| 14208.4620 | 0.089020 | A21 G8 C7 T10 | 14079.4170 | 0.073980 | A15 G11 C17 T3 |
| — | — | — | 14079.4084 | 0.065400 | A20 G2 C5 T19 |
| — | — | — | 14079.4309 | 0.087940 | A20 G5 C10 T13 |

Among the 16 possible base compositions for the forward strand and the 18 possible base compositions for the reverse strand that were calculated, only one pair (shown in bold) are complementary base compositions, which indicates the true base composition of the amplification product. It should be recognized that this logic is applicable for determination of base compositions of any bioagent identifying amplicon, regardless of the class of bioagent from which the corresponding amplification product was obtained.

Assignment of previously unobserved base compositions (also known as “true unknown base compositions”) to a given phylogeny may be accomplished via the use of pattern classifier model algorithms. Base compositions, like sequences, vary slightly from strain to strain within species, for example. In some embodiments, the pattern classifier model is the mutational probability model. On other embodiments, the pattern classifier is the polytope model. The mutational probability model and polytope model are both commonly owned and described in U.S. patent application Ser. No. 11/073,362, the contents of which are incorporated herein by reference in their entirety.

This diversity may be managed by building “base composition probability clouds” around the composition constraints for each species. This permits identification of organisms in a fashion similar to sequence analysis. A “pseudo four-dimensional plot” can be used to visualize the concept of base composition probability clouds. Optimal primer design requires optimal choice of bioagent identifying amplicons and maximizes the separation between the base composition signatures of individual bioagents. Areas where clouds overlap indicate regions that may result in a misclassification, a problem which is overcome by a triangulation identification process using bioagent identifying amplicons not affected by overlap of base composition probability clouds.

Base composition probability clouds may provide the means for screening potential primer pairs in order to avoid potential misclassifications of base compositions. Base composition probability clouds may also provide the means for predicting the identity of a bioagent whose assigned base composition was not previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

The methods disclosed herein provide bioagent classifying information similar to DNA sequencing and phylogenetic analysis at a level sufficient to identify a given bioagent. Furthermore, the process of determination of a previously unknown base composition for a given bioagent (for example, in a case where sequence information is unavailable) has downstream utility by providing additional bioagent indexing information with which to populate base composition databases. The process of future bioagent identification is thus greatly improved as more base composition indexes become available in base composition databases.

### 10. TRIANGULATION IDENTIFICATION

A molecular mass of a single bioagent identifying amplicon alone may not provide enough resolution to unambiguously identify a given bioagent. The employment of more than one bioagent identifying amplicon for identification of a bioagent is herein referred to as “triangulation identification.” Triangulation identification is pursued by determining the molecular masses of a plurality of bioagent identifying amplicons selected within a plurality of housekeeping genes. This process is used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected signatures from the B. anthracis genome would suggest a genetic engineering event.

The triangulation identification process may be pursued by characterization of bioagent identifying amplicons in a massively parallel fashion using the polymerase chain reaction (PCR), such as multiplex PCR where multiple primers are employed in the same amplification reaction mixture, or PCR in multi-well plate format wherein a different and unique pair of primers is used in multiple wells containing otherwise identical reaction mixtures. Such multiplex and multi-well PCR methods are well known to those with ordinary skill in the arts of rapid throughput amplification of nucleic acids. One PCR reaction per well or container may be carried out, followed by an amplicon pooling step wherein the amplification products of different wells are combined in a single well or container which is then subjected to molecular mass analysis. The combination of pooled amplicons can be chosen such that the expected ranges of molecular masses of individual amplicons are not overlapping and thus will not complicate identification of signals.

### 11. CODON BASE COMPOSITION ANALYSIS

One or more nucleotide substitutions within a codon of a gene of an infectious organism may confer drug resistance upon an organism which can be determined by codon base composition analysis. The organism may be a bacterium, virus, fungus or protozoan. The amplification product containing the codon being analyzed may be of a length of about 39 to about 200 nucleobases. The primers employed in obtaining the amplification product can hybridize to upstream and downstream sequences directly adjacent to the codon, or can hybridize to upstream and downstream sequences one or more sequence positions away from the codon. The primers may have between about 70% to 100% sequence complementarity with the sequence of the gene containing the codon being analyzed.

The codon analysis may be undertaken for the purpose of investigating genetic disease in an individual. In other embodiments, the codon analysis is undertaken for the purpose of investigating a drug resistance mutation or any other deleterious mutation in an infectious organism such as a bacterium, virus, fungus or protozoan. In some embodiments, the bioagent is a bacterium identified in a biological product.

The molecular mass of an amplification product containing the codon being analyzed may be measured by mass spectrometry. The mass spectrometry can be either electrospray (ESI) mass spectrometry or matrix-assisted laser desorption ionization (MALDI) mass spectrometry. Time-of-flight (TOF) is an example of one mode of mass spectrometry compatible with the methods disclosed herein.

The methods disclosed herein can also be employed to determine the relative abundance of drug resistant strains of the organism being analyzed. Relative abundances can be calculated from amplitudes of mass spectral signals with relation to internal calibrants. In some embodiments, known quantities of internal amplification calibrants can be included in the amplification reactions and abundances of analyte amplification product estimated in relation to the known quantities of the calibrants.

Upon identification of one or more drug-resistant strains of an infectious organism infecting an individual, one or more alternative treatments may be devised to treat the individual.

### 12. DETERMINATION OF THE QUANTITY OF A BIOAGENT USING A CALIBRATION AMPLICON

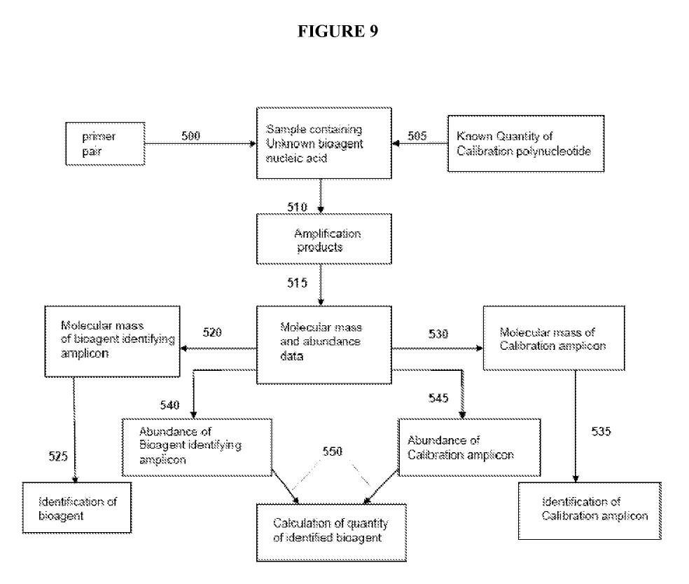

The identity and quantity of an unknown bioagent may be determined using the process illustrated in FIG. 9. Primers (500) and a known quantity of a calibration polynucleotide (505) are added to a sample containing nucleic acid of an unknown bioagent. The total nucleic acid in the sample is then subjected to an amplification reaction (510) to obtain amplification products. The molecular masses of amplification products are determined (515) from which are obtained molecular mass and abundance data. The molecular mass of the bioagent identifying amplicon (520) provides the means for its identification (525) and the molecular mass of the calibration amplicon obtained from the calibration polynucleotide (530) provides the means for its identification (535). The abundance data of the bioagent identifying amplicon is recorded (540) and the abundance data for the calibration data is recorded (545), both of which are used in a calculation (550) which determines the quantity of unknown bioagent in the sample. A sample comprising an unknown bioagent is contacted with a pair of primers that provide the means for amplification of nucleic acid from the bioagent, and a known quantity of a polynucleotide that comprises a calibration sequence. The nucleic acids of the bioagent and of the calibration sequence are amplified and the rate of amplification is reasonably assumed to be similar for the nucleic acid of the bioagent and of the calibration sequence. The amplification reaction then produces two amplification products: a bioagent identifying amplicon and a calibration amplicon. The bioagent identifying amplicon and the calibration amplicon should be distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2-8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent and the abundance data enables calculation of the quantity of the bioagent, based on the knowledge of the quantity of calibration polynucleotide contacted with the sample.

Construction of a standard curve where the amount of calibration polynucleotide spiked into the sample is varied may provide additional resolution and improved confidence for the determination of the quantity of bioagent in the sample. The use of standard curves for analytical determination of molecular quantities is well known to one with ordinary skill and can be performed without undue experimentation.

Multiplex amplification may be performed where multiple bioagent identifying amplicons are amplified with multiple primer pairs which also amplify the corresponding standard calibration sequences. The standard calibration sequences may optionally be included within a single vector which functions as the calibration polynucleotide. Multiplex amplification methods are well known to those with ordinary skill and can be performed without undue experimentation.

The calibrant polynucleotide may be used as an internal positive control to confirm that amplification conditions and subsequent analysis steps are successful in producing a measurable amplicon. Even in the absence of copies of the genome of a bioagent, the calibration polynucleotide should give rise to a calibration amplicon. Failure to produce a measurable calibration amplicon indicates a failure of amplification or subsequent analysis step such as amplicon purification or molecular mass determination. Reaching a conclusion that such failures have occurred is in itself, a useful event.

The calibration sequence may be comprised of DNA or RNA. The calibration sequence may be inserted into a vector that itself functions as the calibration polynucleotide. More than one calibration sequence may be inserted into the vector that functions as the calibration polynucleotide. Such a calibration polynucleotide is herein termed a “combination calibration polynucleotide.” The process of inserting polynucleotides into vectors is routine to those skilled in the art and can be accomplished without undue experimentation. Thus, it should be recognized that the calibration method should not be limited to the embodiments described herein. The calibration method can be applied for determination of the quantity of any bioagent identifying amplicon when an appropriate standard calibrant polynucleotide sequence is designed and used. The process of choosing an appropriate vector for insertion of a calibrant is also a routine operation that can be accomplished by one with ordinary skill without undue experimentation.

### 13. IDENTIFICATION OF BACTERIA USING BIOAGENT IDENTIFYING AMPLICONS

The primer pairs may produce bioagent identifying amplicons defined by priming regions at stable and highly conserved regions of nucleic acid of bacteria. The advantage to characterization of an amplicon defined by priming regions that fall within a highly conserved region is that there is a low probability that the region will evolve past the point of primer recognition, in which case, the primer hybridization of the amplification step would fail. Such a primer pair is thus useful as a broad range survey-type primer pair. In another embodiment, the intelligent primers produce bioagent identifying amplicons including a region which evolves more quickly than the stable region described above. The advantage of characterization bioagent identifying amplicon corresponding to an evolving genomic region is that it is useful for distinguishing emerging strain variants or the presence of virulence genes, drug resistance genes, or codon mutations that induce drug resistance.

The methods disclosed herein have significant advantages as a platform for identification of diseases caused by emerging bacterial strains such as, for example, drug-resistant strains of Staphylococcus aureus. The methods disclosed herein eliminate the need for prior knowledge of bioagent sequence to generate hybridization probes. This is possible because the methods are not confounded by naturally occurring evolutionary variations occurring in the sequence acting as the template for production of the bioagent identifying amplicon. Measurement of molecular mass and determination of base composition is accomplished in an unbiased manner without sequence prejudice.

Provided herein is a means of tracking the spread of a bacterium, such as a particular drug-resistant strain when a plurality of samples obtained from different locations are analyzed by the methods described above in an epidemiological setting. A plurality of samples from a plurality of different locations may be analyzed with primer pairs which produce bioagent identifying amplicons, a subset of which contains a specific drug-resistant bacterial strain. The corresponding locations of the members of the drug-resistant strain subset indicate the spread of the specific drug-resistant strain to the corresponding locations.

Also provided is a means of identifying a sepsis-causing bacterium. The sepsis-causing bacterium is identified in samples including, but not limited to blood and fractions thereof (including but not limited to serum and buffy coat), sputum, urine, specific cell types including but not limited to hepatic cells, and various tissue biopsies.

Sepsis-causing bacteria include, but are not limited to the following bacteria: Prevotella denticola, Porphyromonas gingivalis, Borrelia burgdorferi, Mycobacterium tuberculosis, Mycobacterium fortuitum, Corynebacterium jeikeium, Propionibacterium acnes, Mycoplasma pneumoniae, Streptococcus agalactiae, Streptococcus pneumoniae, Streptococcus mitis, Streptococcus pyogenes, Listeria monocytogenes, Enterococcus faecalis, Enterococcus faecium, Staphylococcus aureus, Staphylococcus coagulase-negative, Staphylococcus epidermis, Staphylococcus hemolyticus, Campylobacter jejuni, Bordatella pertussis, Burkholderia cepacia, Legionella pneumophila, Acinetobacter baumannii, Acinetobacter calcoaceticus, Pseudomonas aeru ginosa, Aeromonas hydrophila, Enterobacter aerogenes, Enterobacter cloacae, Klebsiella pneumoniae, Moxarella catarrhalis, Morganella morganii, Proteus mirabilis, Proteus vulgaris, Pantoea agglomerans, Bartonella henselae, Stenotrophomonas maltophila, Actinobacillus actinomycetemcomitans, Haemophilus influenzae, Escherichia coli, Klebsiella oxytoca, Serratia marcescens, and Yersinia enterocolitica.

Identification of a sepsis-causing bacterium may provide the information required to choose an antibiotic with which to treat an individual infected with the sepsis-causing bacterium and treating the individual with the antibiotic. Treatment of humans with antibiotics is well known to medical practitioners with ordinary skill.

### 14. KITS FOR PRODUCING BIOAGENT IDENTIFYING AMPLICONS

Also provided are kits for carrying out the methods described herein. In some embodiments, the kit may comprise a sufficient quantity of one or more primer pairs to perform an amplification reaction on a target polynucleotide from a bioagent to form a bioagent identifying amplicon. The kit may comprise from one to fifty primer pairs, from one to twenty primer pairs, from one to ten primer pairs, or from two to five primer pairs. The kit may comprise one or more primer pairs recited in Table 2 of U.S. Ser. No. 11/409,535, the contents of which are incorporated herein by reference in their entirety.

The kit may comprise one or more broad range survey primer(s), division wide primer(s), or drill-down primer(s), or any combination thereof. If a given problem involves identification of a specific bioagent, the solution to the problem may require the selection of a particular combination of primers to provide the solution to the problem. A kit may be designed so as to comprise particular primer pairs for identification of a particular bioagent. A drill-down kit may be used, for example, to distinguish different genotypes or strains, drug-resistant, or otherwise. The primer pair components of any of these kits may be additionally combined to comprise additional combinations of broad range survey primers and division-wide primers so as to be able to identify a bacterium.

The kit may contain standardized calibration polynucleotides for use as internal amplification calibrants. Internal calibrants are described in commonly owned PCT Publication No. WO 2005/098047, the contents of which are incorporated herein by reference in their entirety.

The kit may comprise a sufficient quantity of reverse transcriptase (if RNA is to be analyzed for example), a DNA polymerase, suitable nucleoside triphosphates (including alternative dNTPs such as inosine or modified dNTPs such as the 5-propynyl pyrimidines or any dNTP containing molecular mass-modifying tags such as those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. A kit may also comprise amplification reaction containers such as microcentrifuge tubes and the like. A kit may also comprise reagents or other materials for isolating bioagent nucleic acid or bioagent identifying amplicons from amplification, including, for example, detergents, solvents, or ion exchange resins which may be linked to magnetic beads. A kit may also comprise a table of measured or calculated molecular masses and/or base compositions of bioagents using the primer pairs of the kit.

Also provided is a kit that contains one or more survey bacterial primer pairs represented by primer pair compositions wherein each member of each pair of primers has 70% to 100% sequence identity with the corresponding member from the group of primer pairs represented by any of the primer pairs of Table 2 of U.S. Ser. No. 11/409,535. The survey primer pairs may include broad range primer pairs which hybridize to ribosomal RNA, and may also include division-wide primer pairs which hybridize to housekeeping genes such as rp1B, tufB, rpoB, rpoC, valS, and infB, for example.

The kit may contain one or more survey bacterial primer pairs and one or more triangulation genotyping analysis primer pairs such as the primer pairs of Tables 8, 12, 14, 19, 21, 23, or 24 of U.S. Ser. No. 11/409,535. The kit may represent a less expansive genotyping analysis but include triangulation genotyping analysis primer pairs for more than one genus or species of bacteria. For example, a kit for surveying nosocomial infections at a health care facility may include, for example, one or more broad range survey primer pairs, one or more division wide primer pairs, one or more Acinetobacter baumannii triangulation genotyping analysis primer pairs and one or more Staphylococcus aureus triangulation genotyping analysis primer pairs. One with ordinary skill will be capable of analyzing in silico amplification data to determine which primer pairs will be able to provide optimal identification resolution for the bacterial bioagents of interest.

A kit may be assembled for identification of sepsis-causing bacteria. An example of such a kit embodiment is a kit comprising one or more of the primer pairs of Table 25 of U.S. Ser. No. 11/409,535, which provide for a broad survey of sepsis-causing bacteria.

The kit may have 96-well or 384-well plates with a plurality of wells containing any or all of the following components: dNTPs, buffer salts, Mg²⁺, betaine, and primer pairs. A polymerase may also be included in the plurality of wells of the 96-well or 384-well plates. The kit may contain instructions for PCR and mass spectrometry analysis of amplification products obtained using the primer pairs of the kits. The kit may include a barcode which uniquely identifies the kit and the components contained therein according to production lots and may also include any other information relative to the components such as concentrations, storage temperatures, etc. The barcode may also include analysis information to be read by optical barcode readers and sent to a computer controlling amplification, purification and mass spectrometric measurements. The barcode may provide access to a subset of base compositions in a base composition database which is in digital communication with base composition analysis software such that a base composition measured with primer pairs from a given kit can be compared with known base compositions of bioagent identifying amplicons defined by the primer pairs of that kit.

The kit may contain a database of base compositions of bioagent identifying amplicons defined by the primer pairs of the kit. The database is stored on a convenient computer readable medium such as a compact disk or USB drive, for example.

The kit may include a computer program stored on a computer formatted medium (such as a compact disk or portable USB disk drive, for example) comprising instructions which direct a processor to analyze data obtained from the use of the primer pairs disclosed herein. The instructions of the software transform data related to amplification products into a molecular mass or base composition which is a useful concrete and tangible result used in identification and/or classification of bioagents. The kit may contain all of the reagents sufficient to carry out one or more of the methods described herein.

### 15. COMBINATION KITS INCLUDING TARGETED GENOME AMPLIFICATION PRIMERS AND PRIMER PAIRS FOR OBTAINING BIOAGENT IDENTIFYING AMPLICONS

Also provided herein is a kit that includes targeted genome amplification primers and primer pairs for production of bioagent identifying amplicons. The kit may be for use in applications where a bioagent such as a human pathogen for example, is present only in small quantities in a human clinical sample. An example of such a kit could include a set of targeted genome amplification primers for selective amplification of a bacterium implicated in septicemia. The targeted genome amplification primers are designed with human genomic DNA chosen as a background genome, for the purpose of detection of an infection of an individual with Bacillus anthracis. The kit would also include one or more broad range survey primer pairs and/or division-wide primer pairs for production of amplification products corresponding to bioagent identifying amplicons for identification of the bacterium. Optionally one or more drill-down primer pairs are included in the kit for determining sub-species characteristics of the septicemia by analysis of additional bioagent identifying amplicons.

The combination kit may also include a plurality of polymerase enzymes whose members are specialized for a PCR type amplification reaction, such as Taq polymerase, for example, to obtain amplification products corresponding to bioagent identifying amplicons, and such as Phi29 polymerase which is a high processivity polymerase suitable for catalysis of multiple displacement amplification reactions for targeted genome amplification reactions carried out for elevating the quantity of a target genome of interest.

The combination kit may also include amplification reagents including but not limited to: deoxynucleotide triphosphates, compatible solutes such as betaine and trehalose, buffer components, and salts such as magnesium chloride.

While the present invention has been described with specificity in accordance with certain of its embodiments, the following examples serve only to illustrate the invention and are not intended to limit the same. In order that the invention disclosed herein may be more efficiently understood, examples are provided below. It should be understood that these examples are for illustrative purposes only and are not to be construed as limiting the invention in any manner.

The present invention has multiple aspects, illustrated by the following non-limiting examples.

### Example 1

### Identification and Ranking of Genome Sequence Segments

This example illustrates the process of identification of unique genome sequence segments of 6 to 12 nucleobases in length, as well as determination of frequency of occurrence and selectivity ratio values for a simplified hypothetical genome model system consisting of a single target genome having the sequence: aaaaaaaaaattttttttttccccccccccgggggggggg ((SEQ ID NO: 16) base composition of A10 T10 C10 and G10) with two background genomes having the following sequences aaaaaaaattttttttccccccccgggggggg (SEQ ID NO: 17) Bkg 1: base composition of A8 T8 C8 G8) and aaaaaaaaaatttttttttt (SEQ ID NO: 18) Bkg 2: base composition of A10 T10 C0 G0). Table 2 provides a list of all unique genome sequence segments for the target genome and indicates the frequency of occurrence of each genome sequence segment in the target genome and in the background genomes. For example, the genome sequence segment having the sequence of eight consecutive c residues cccccccc (SEQ ID NO:45) occurs 3 times (bold) within the 10 nucleobase stretch of c residues in the simplified hypothetical target genome:

(SEQ ID NO: 16)
aaaaaaaaaattttttttttccccccccccgggggggggg;
(SEQ ID NO: 16)
aaaaaaaaaattttttttttccccccccccgggggggggg;
(SEQ ID NO: 16)
aaaaaaaaaattttttttttccccccccccgggggggggg;

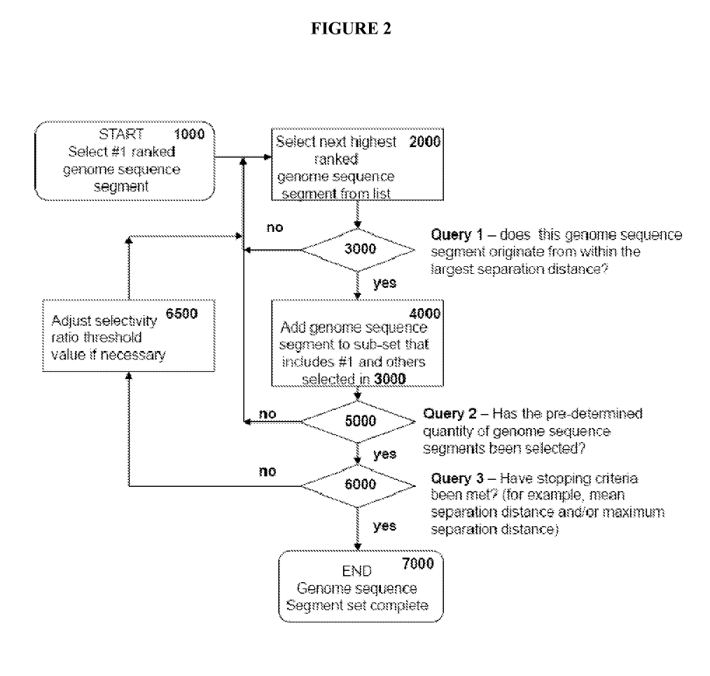

(c residue stretch underlined) but only once in the background genomes (the genome sequence segment appears once in Bkg 1 and does not appear in Bkg 2). The selectivity ratio for this genome sequence segment is 3.00 as determined by dividing the frequency of occurrence in the target genome by the frequency of occurrence in the background genomes. The data in Table 2 are sorted according to the selectivity ratio rank. A selectivity ratio of infinity (.infin.) indicates that the genome sequence segment does not occur in the background genomes (Bkg 1 and Bkg 2). The mean frequency of occurrence of the genome sequence segments in the target genome was calculated to be 1.22 and the mean selectivity ratio was calculated to be 0.76. If desired, these values could be used as threshold values for selection of one or more sub-sets of genome sequence segments for further characterization by processes such as the process shown in FIG. 2 for example. Alternatively, threshold values greater than or less than the mean frequency of occurrence or the mean selectivity ratio could be chosen.

TABLE 2
Frequency of Occurrence of Genome Sequence Segments in a Hypothetical Target
Genome and Two Hypothetical Background Genomes

| Genome | | | | | | Selec- | Selec- |
|---|---|---|---|---|---|---|---|
| Sequence | SEQ ID | Frequency | Frequency | Frequency | Total | tivity | tivity |
| Segment | NO: | in Target | in Bkg 1 | in Bkg 2 | Background | Ratio | Ratio Rank |
| ccccccccc | 19 | 2 | 0 | 0 | 0 | Infinity | 1 |
| ggggggggg | 20 | 2 | 0 | 0 | 0 | Infinity | 1 |
| cccccccccc | 21 | 1 | 0 | 0 | 0 | Infinity | 1 |
| cccccccccg | 22 | 1 | 0 | 0 | 0 | Infinity | 1 |
| cggggggggg | 23 | 1 | 0 | 0 | 0 | Infinity | 1 |
| gggggggggg | 24 | 1 | 0 | 0 | 0 | Infinity | 1 |
| tccccccccc | 25 | 1 | 0 | 0 | 0 | Infinity | 1 |
| tttttttttc | 26 | 1 | 0 | 0 | 0 | Infinity | 1 |
| ccccccccccg | 27 | 1 | 0 | 0 | 0 | Infinity | 1 |
| cccccccccgg | 28 | 1 | 0 | 0 | 0 | Infinity | 1 |
| ccggggggggg | 29 | 1 | 0 | 0 | 0 | Infinity | 1 |
| cgggggggggg | 30 | 1 | 0 | 0 | 0 | Infinity | 1 |
| tcccccccccc | 31 | 1 | 0 | 0 | 0 | Infinity | 1 |
| ttccccccccc | 32 | 1 | 0 | 0 | 0 | Infinity | 1 |
| tttttttttcc | 33 | 1 | 0 | 0 | 0 | Infinity | 1 |
| ttttttttttc | 34 | 1 | 0 | 0 | 0 | Infinity | 1 |
| attttttttttc | 35 | 1 | 0 | 0 | 0 | Infinity | 1 |
| ccccccccccgg | 36 | 1 | 0 | 0 | 0 | Infinity | 1 |
| cccccccccggg | 37 | 1 | 0 | 0 | 0 | Infinity | 1 |
| cccggggggggg | 38 | 1 | 0 | 0 | 0 | Infinity | 1 |
| ccgggggggggg | 39 | 1 | 0 | 0 | 0 | Infinity | 1 |
| tccccccccccg | 40 | 1 | 0 | 0 | 0 | Infinity | 1 |
| ttcccccccccc | 41 | 1 | 0 | 0 | 0 | Infinity | 1 |
| tttccccccccc | 42 | 1 | 0 | 0 | 0 | Infinity | 1 |
| tttttttttccc | 43 | 1 | 0 | 0 | 0 | Infinity | 1 |
| ttttttttttcc | 44 | 1 | 0 | 0 | 0 | Infinity | 1 |
| cccccccc | 45 | 3 | 1 | 0 | 1 | 3.00 | 2 |
| gggggggg | 46 | 3 | 1 | 0 | 1 | 3.00 | 2 |
| ggggggg | 47 | 4 | 2 | 0 | 2 | 2.00 | 3 |
| cccccc | 48 | 5 | 3 | 0 | 3 | 1.67 | 4 |
| gggggg | 49 | 5 | 3 | 0 | 3 | 1.67 | 4 |
| cccccg | 50 | 1 | 1 | 0 | 1 | 1.00 | 5 |
| ccccgg | 51 | 1 | 1 | 0 | 1 | 1.00 | 5 |
| cccggg | 52 | 1 | 1 | 0 | 1 | 1.00 | 5 |
| ccgggg | 53 | 1 | 1 | 0 | 1 | 1.00 | 5 |
| cggggg | 54 | 1 | 1 | 0 | 1 | 1.00 | 5 |
| tccccc | 55 | 1 | 1 | 0 | 1 | 1.00 | 5 |
| ttcccc | 56 | 1 | 1 | 0 | 1 | 1.00 | 5 |
| tttccc | 57 | 1 | 1 | 0 | 1 | 1.00 | 5 |
| ttttcc | 58 | 1 | 1 | 0 | 1 | 1.00 | 5 |
| tttttc | 59 | 1 | 1 | 0 | 1 | 1.00 | 5 |
| ccccccg | 60 | 1 | 1 | 0 | 1 | 1.00 | 5 |
| cccccgg | 61 | 1 | 1 | 0 | 1 | 1.00 | 5 |
| ccccggg | 62 | 1 | 1 | 0 | 1 | 1.00 | 5 |
| cccgggg | 63 | 1 | 1 | 0 | 1 | 1.00 | 5 |
| ccggggg | 64 | 1 | 1 | 0 | 1 | 1.00 | 5 |
| cgggggg | 65 | 1 | 1 | 0 | 1 | 1.00 | 5 |
| tcccccc | 66 | 1 | 1 | 0 | 1 | 1.00 | 5 |
| ttccccc | 67 | 1 | 1 | 0 | 1 | 1.00 | 5 |
| tttcccc | 68 | 1 | 1 | 0 | 1 | 1.00 | 5 |
| ttttccc | 69 | 1 | 1 | 0 | 1 | 1.00 | 5 |
| tttttcc | 70 | 1 | 1 | 0 | 1 | 1.00 | 5 |
| ttttttc | 71 | 1 | 1 | 0 | 1 | 1.00 | 5 |
| cccccccg | 72 | 1 | 1 | 0 | 1 | 1.00 | 5 |
| ccccccgg | 73 | 1 | 1 | 0 | 1 | 1.00 | 5 |
| cccccggg | 74 | 1 | 1 | 0 | 1 | 1.00 | 5 |
| ccccgggg | 75 | 1 | 1 | 0 | 1 | 1.00 | 5 |
| cccggggg | 76 | 1 | 1 | 0 | 1 | 1.00 | 5 |
| ccgggggg | 77 | 1 | 1 | 0 | 1 | 1.00 | 5 |
| cggggggg | 78 | 1 | 1 | 0 | 1 | 1.00 | 5 |
| tccccccc | 79 | 1 | 1 | 0 | 1 | 1.00 | 5 |
| ttcccccc | 80 | 1 | 1 | 0 | 1 | 1.00 | 5 |
| tttccccc | 81 | 1 | 1 | 0 | 1 | 1.00 | 5 |
| ttttcccc | 82 | 1 | 1 | 0 | 1 | 1.00 | 5 |
| tttttccc | 83 | 1 | 1 | 0 | 1 | 1.00 | 5 |
| ttttttcc | 84 | 1 | 1 | 0 | 1 | 1.00 | 5 |
| tttttttc | 85 | 1 | 1 | 0 | 1 | 1.00 | 5 |
| aaaaaaaaa | 86 | 2 | 0 | 2 | 2 | 1.00 | 5 |
| ccccccccg | 87 | 1 | 1 | 0 | 1 | 1.00 | 5 |
| cccccccgg | 88 | 1 | 1 | 0 | 1 | 1.00 | 5 |
| ccccccggg | 89 | 1 | 1 | 0 | 1 | 1.00 | 5 |
| cccccgggg | 90 | 1 | 1 | 0 | 1 | 1.00 | 5 |
| ccccggggg | 91 | 1 | 1 | 0 | 1 | 1.00 | 5 |
| cccgggggg | 92 | 1 | 1 | 0 | 1 | 1.00 | 5 |
| ccggggggg | 93 | 1 | 1 | 0 | 1 | 1.00 | 5 |
| cgggggggg | 94 | 1 | 1 | 0 | 1 | 1.00 | 5 |
| tcccccccc | 95 | 1 | 1 | 0 | 1 | 1.00 | 5 |
| ttccccccc | 96 | 1 | 1 | 0 | 1 | 1.00 | 5 |
| tttcccccc | 97 | 1 | 1 | 0 | 1 | 1.00 | 5 |
| ttttccccc | 98 | 1 | 1 | 0 | 1 | 1.00 | 5 |
| tttttcccc | 99 | 1 | 1 | 0 | 1 | 1.00 | 5 |
| ttttttccc | 100 | 1 | 1 | 0 | 1 | 1.00 | 5 |
| tttttttcc | 101 | 1 | 1 | 0 | 1 | 1.00 | 5 |
| ttttttttc | 102 | 1 | 1 | 0 | 1 | 1.00 | 5 |
| ttttttttt | 103 | 2 | 0 | 2 | 2 | 1.00 | 5 |
| aaaaaaaaaa | 104 | 1 | 0 | 1 | 1 | 1.00 | 5 |
| aaaaaaaaat | 105 | 1 | 0 | 1 | 1 | 1.00 | 5 |
| attttttttt | 106 | 1 | 0 | 1 | 1 | 1.00 | 5 |
| ccccccccgg | 107 | 1 | 1 | 0 | 1 | 1.00 | 5 |
| cccccccggg | 108 | 1 | 1 | 0 | 1 | 1.00 | 5 |
| ccccccgggg | 109 | 1 | 1 | 0 | 1 | 1.00 | 5 |
| cccccggggg | 110 | 1 | 1 | 0 | 1 | 1.00 | 5 |
| ccccgggggg | 111 | 1 | 1 | 0 | 1 | 1.00 | 5 |
| cccggggggg | 112 | 1 | 1 | 0 | 1 | 1.00 | 5 |
| ccgggggggg | 113 | 1 | 1 | 0 | 1 | 1.00 | 5 |
| ttcccccccc | 114 | 1 | 1 | 0 | 1 | 1.00 | 5 |
| tttccccccc | 115 | 1 | 1 | 0 | 1 | 1.00 | 5 |
| ttttcccccc | 116 | 1 | 1 | 0 | 1 | 1.00 | 5 |
| tttttccccc | 117 | 1 | 1 | 0 | 1 | 1.00 | 5 |
| ttttttcccc | 118 | 1 | 1 | 0 | 1 | 1.00 | 5 |
| tttttttccc | 119 | 1 | 1 | 0 | 1 | 1.00 | 5 |
| ttttttttcc | 120 | 1 | 1 | 0 | 1 | 1.00 | 5 |
| tttttttttt | 121 | 1 | 0 | 1 | 1 | 1.00 | 5 |
| aaaaaaaaaat | 122 | 1 | 0 | 1 | 1 | 1.00 | 5 |
| aaaaaaaaatt | 123 | 1 | 0 | 1 | 1 | 1.00 | 5 |
| aattttttttt | 124 | 1 | 0 | 1 | 1 | 1.00 | 5 |
| atttttttttt | 125 | 1 | 0 | 1 | 1 | 1.00 | 5 |
| ccccccccggg | 126 | 1 | 1 | 0 | 1 | 1.00 | 5 |
| cccccccgggg | 127 | 1 | 1 | 0 | 1 | 1.00 | 5 |
| ccccccggggg | 128 | 1 | 1 | 0 | 1 | 1.00 | 5 |
| cccccgggggg | 129 | 1 | 1 | 0 | 1 | 1.00 | 5 |
| ccccggggggg | 130 | 1 | 1 | 0 | 1 | 1.00 | 5 |
| cccgggggggg | 131 | 1 | 1 | 0 | 1 | 1.00 | 5 |
| tttcccccccc | 132 | 1 | 1 | 0 | 1 | 1.00 | 5 |
| ttttccccccc | 133 | 1 | 1 | 0 | 1 | 1.00 | 5 |
| tttttcccccc | 134 | 1 | 1 | 0 | 1 | 1.00 | 5 |
| ttttttccccc | 135 | 1 | 1 | 0 | 1 | 1.00 | 5 |
| tttttttcccc | 136 | 1 | 1 | 0 | 1 | 1.00 | 5 |
| ttttttttccc | 137 | 1 | 1 | 0 | 1 | 1.00 | 5 |
| aaaaaaaaaatt | 138 | 1 | 0 | 1 | 1 | 1.00 | 5 |
| aaaaaaaaattt | 139 | 1 | 0 | 1 | 1 | 1.00 | 5 |
| aaattttttttt | 140 | 1 | 0 | 1 | 1 | 1.00 | 5 |
| aatttttttttt | 141 | 1 | 0 | 1 | 1 | 1.00 | 5 |
| ccccccccgggg | 142 | 1 | 1 | 0 | 1 | 1.00 | 5 |
| cccccccggggg | 143 | 1 | 1 | 0 | 1 | 1.00 | 5 |
| ccccccgggggg | 144 | 1 | 1 | 0 | 1 | 1.00 | 5 |
| cccccggggggg | 145 | 1 | 1 | 0 | 1 | 1.00 | 5 |
| ccccgggggggg | 146 | 1 | 1 | 0 | 1 | 1.00 | 5 |
| ttttcccccccc | 147 | 1 | 1 | 0 | 1 | 1.00 | 5 |
| tttttccccccc | 148 | 1 | 1 | 0 | 1 | 1.00 | 5 |
| ttttttcccccc | 149 | 1 | 1 | 0 | 1 | 1.00 | 5 |
| tttttttccccc | 150 | 1 | 1 | 0 | 1 | 1.00 | 5 |
| ttttttttcccc | 151 | 1 | 1 | 0 | 1 | 1.00 | 5 |
| aaaaaaaa | 15 | 3 | 1 | 3 | 4 | 0.75 | 6 |
| tttttttt | 153 | 3 | 1 | 3 | 4 | 0.75 | 6 |
| aaaaaaa | 154 | 4 | 2 | 4 | 6 | 0.67 | 7 |
| ccccccc | 155 | 4 | 2 | 4 | 6 | 0.67 | 7 |
| ttttttt | 156 | 4 | 2 | 4 | 6 | 0.67 | 7 |
| aaaaaa | 157 | 5 | 3 | 5 | 8 | 0.63 | 8 |
| tttttt | 158 | 5 | 3 | 5 | 8 | 0.63 | 8 |
| aaaaat | 159 | 1 | 1 | 1 | 2 | 0.50 | 9 |
| aaaatt | 160 | 1 | 1 | 1 | 2 | 0.50 | 9 |
| aaattt | 161 | 1 | 1 | 1 | 2 | 0.50 | 9 |
| aatttt | 162 | 1 | 1 | 1 | 2 | 0.50 | 9 |
| attttt | 163 | 1 | 1 | 1 | 2 | 0.50 | 9 |
| aaaaaat | 164 | 1 | 1 | 1 | 2 | 0.50 | 9 |
| aaaaatt | 165 | 1 | 1 | 1 | 2 | 0.50 | 9 |
| aaaattt | 166 | 1 | 1 | 1 | 2 | 0.50 | 9 |
| aaatttt | 167 | 1 | 1 | 1 | 2 | 0.50 | 9 |
| aattttt | 168 | 1 | 1 | 1 | 2 | 0.50 | 9 |
| atttttt | 169 | 1 | 1 | 1 | 2 | 0.50 | 9 |
| aaaaaaat | 170 | 1 | 1 | 1 | 2 | 0.50 | 9 |
| aaaaaatt | 171 | 1 | 1 | 1 | 2 | 0.50 | 9 |
| aaaaattt | 172 | 1 | 1 | 1 | 2 | 0.50 | 9 |
| aaaatttt | 173 | 1 | 1 | 1 | 2 | 0.50 | 9 |
| aaattttt | 174 | 1 | 1 | 1 | 2 | 0.50 | 9 |
| aatttttt | 175 | 1 | 1 | 1 | 2 | 0.50 | 9 |
| attttttt | 176 | 1 | 1 | 1 | 2 | 0.50 | 9 |
| aaaaaaaat | 177 | 1 | 1 | 1 | 2 | 0.50 | 9 |
| aaaaaaatt | 178 | 1 | 1 | 1 | 2 | 0.50 | 9 |
| aaaaaattt | 179 | 1 | 1 | 1 | 2 | 0.50 | 9 |
| aaaaatttt | 180 | 1 | 1 | 1 | 2 | 0.50 | 9 |
| aaaattttt | 181 | 1 | 1 | 1 | 2 | 0.50 | 9 |
| aaatttttt | 182 | 1 | 1 | 1 | 2 | 0.50 | 9 |
| aattttttt | 183 | 1 | 1 | 1 | 2 | 0.50 | 9 |
| atttttttt | 184 | 1 | 1 | 1 | 2 | 0.50 | 9 |
| aaaaaaaatt | 185 | 1 | 1 | 1 | 2 | 0.50 | 9 |
| aaaaaaattt | 186 | 1 | 1 | 1 | 2 | 0.50 | 9 |
| aaaaaatttt | 187 | 1 | 1 | 1 | 2 | 0.50 | 9 |
| aaaaattttt | 188 | 1 | 1 | 1 | 2 | 0.50 | 9 |
| aaaatttttt | 189 | 1 | 1 | 1 | 2 | 0.50 | 9 |
| aaattttttt | 190 | 1 | 1 | 1 | 2 | 0.50 | 9 |
| aatttttttt | 191 | 1 | 1 | 1 | 2 | 0.50 | 9 |
| aaaaaaaattt | 192 | 1 | 1 | 1 | 2 | 0.50 | 9 |
| aaaaaaatttt | 193 | 1 | 1 | 1 | 2 | 0.50 | 9 |
| aaaaaattttt | 194 | 1 | 1 | 1 | 2 | 0.50 | 9 |
| aaaaatttttt | 195 | 1 | 1 | 1 | 2 | 0.50 | 9 |
| aaaattttttt | 196 | 1 | 1 | 1 | 2 | 0.50 | 9 |
| aaatttttttt | 197 | 1 | 1 | 1 | 2 | 0.50 | 9 |
| aaaaaaaatttt | 198 | 1 | 1 | 1 | 2 | 0.50 | 9 |
| aaaaaaattttt | 199 | 1 | 1 | 1 | 2 | 0.50 | 9 |
| aaaaaatttttt | 200 | 1 | 1 | 1 | 2 | 0.50 | 9 |
| aaaaattttttt | 201 | 1 | 1 | 1 | 2 | 0.50 | 9 |
| aaaatttttttt | 202 | 1 | 1 | 1 | 2 | 0.50 | 9 |

### Example 2

### In Silico Method for Design of Primers for Targeted Whole Genome Amplification

Some embodiments of the methods disclosed herein are in silico methods for selecting primers for targeted whole genome amplification. The primers are selected by first defining the target genome(s) and background genome(s). For the target genome(s), all unique genome sequence segments of lengths of about 5 to about 13 nucleobases in length are determined by a set of computer executable instructions stored on a computer-readable medium.

In some embodiments, the target and background genome segments are obtained from public databases such as GenBank, for example. The frequency of occurrence values of members of the genome sequence segments in the target genome(s) and background genome(s) are determined by computer executable instructions such as a BLAST algorithm for example. The selectivity ratio values of members of the genome sequence segments are determined by computer executable mathematical instructions. In some embodiments, the in silico method ranks the genome sequence segments according to frequency of occurrence and/or selectivity ratio. In some embodiments, a frequency of occurrence threshold value is chosen to define a sub-set of genome sequence segments to carry forward.

In some embodiments, a selectivity ratio threshold value is chosen to define a sub-set of genome sequence segments to carry forward. In some embodiments, the selectivity ratio threshold value is any whole or fractional percentage between about 25% above or about 25% below the mean selectivity ratio. For example, if the mean selectivity ratio is 55, the chosen selectivity ratio threshold value may be any whole or fractional number between about 41.25 and about 68.75. In other embodiments, both a frequency of occurrence threshold value and a selectivity ratio threshold value are chosen and both of these threshold values are used to define the sub-set of genome sequence segments to carry forward. The genome sequence segments are ranked according to the chosen threshold value.

At this point, a process such as the process outlined in FIG. 2 may be followed wherein the top ranked genome sequence segment is selected and added to the sub-set of genome sequence segments (1000). Then the next highest ranking genome sequence segment is selected (2000) and subjected to a first computer executable query (3000) which determines whether or not the next ranked genome sequence segment originates from within the largest remaining separation distance (remaining portion of the genome which has not had a genome sequence segment selected). If the next highest ranking genome sequence segment does not originate within the largest separation distance, it is skipped (but remains in with the same rank in the group of unselected genome sequence segments) and the process reverts to step 2000. If the next highest ranking genome sequence segment does originate from within the largest separation distance it is selected and added to the set of genome sequence segments to which primers will be designed (4000). An example of operation of steps 1000 to 5000 (including cycling between steps 2000 and 5000) of FIG. 2 follows: the top ranked genome sequence segment (#1) is selected by default in step 1000. As a result of selection of genome sequence segment #1, only two separation distances remain on the target genome. One of the two separation distances stretches from the 5′ end of the #1 genome sequence segment to the 5′ end of the genome and the other of the two separation distances stretches from the 3′ end of the #1 genome sequence segment to the 5′ end of the genome. It is assumed in this example that the 5′ end of the genome to the 5′ end of the #1 genome sequence segment has the longest separation distance. In step 2000, the next highest ranked genome sequence segment (#2 in this case) is selected. At step 3000 (query 1) it is determined whether or not the #2 ranked genome sequence segment is located within this longest separation distance between the 5′ end of the genome and the 5′ end of the #1 genome sequence segment. If the #2 ranked genome sequence segment is not located within this longest separation distance, it is not selected and remains in the unselected group while the process reverts to step 2000 where the next highest ranked genome sequence segment (#3) is selected from the list of ranked genome sequence segments. In performing step 3000 on genome sequence segment #3, it is determined that this genome sequence segment is located within the largest separation distance. Thus genome sequence segment #3 is added to the sub-set in step 4000. At this point, only genome sequence segments #1 and #3 have been added to the sub-set. In step 5000, it is confirmed that the predetermined quantity of genome sequence segments (for example 200 genome sequence segments) has not been obtained (because only 2 genome sequence segments have been selected thus far). The answer to query 2 (5000) is “no” and the process cycles back to step 2000 where the next ranked genome sequence segment is selected. In this example, the next ranked genome sequence segment is #2 because it was skipped in the previous cycle. In step 3000 query 1 determines that genome sequence segment now does fall within the largest separation distance (because the largest separation distance in the previous cycle is no longer the largest in the current cycle due to the appearance of genome sequence segment #3). Thus genome sequence segment #2 is added to the sub-set in step 4000. Step 5000 is then performed and the answer to query 2 is “no” because only 3 genome sequence segments have been selected thus far. Again the process cycles back to step 2000 and continues cycling between steps 2000 and 5000, selecting the next highest ranked genome sequence segments in each cycle and performing the queries of step 3000 and step 5000 until the predetermined quantity of genome sequence segments is obtained.

In some embodiments, the predetermined number of genome sequence segments is sufficient to provide consistently dispersed coverage of the genome by primers hybridizing to the selected genome sequence segments. In some embodiments, this predetermined number of genome sequence segments is between about 100 to about 300 genome sequence segments, including any number therebetween.

The predetermined number will depend upon the length of the target genome(s). For example, longer genomes may require additional primer coverage and thus selecting a larger predetermined number of genome sequence segments to serve as primer hybridization sites may be advantageous. In some embodiments, after a group of genome sequence segments have been selected, statistical measures such as those presented in Table 5 may be used to evaluate the likelihood that a group of primers designed to hybridize to the genome sequence segments will produce efficient and biased amplification of the target genome(s) of interest. If the statistics are deemed inefficient, it may be advantageous to consider revising the predetermined number of genome sequence segments to a larger number to provide greater coverage of the target genome(s). This statistical evaluation process is useful because it avoids the unnecessary expense of in vitro testing of entire groups of primers.

Continuing now in the process of FIG. 2, when the answer to the second query (5000) is “yes,” the predetermined quantity of genome sequence segments has been obtained. At that point, a third computer executable query (6000) is performed to determine whether or not the “stopping criterion/criteria” has or have been met. The “stopping criterion/criteria” represent the final threshold value(s) relating to genome sequence segment coverage over which the in silico method must pass before the method instructions and queries of the in silico end (7000). If the stopping criteria have not been met, the process cycles back to step 2000 with an adjustment of the selectivity threshold value if necessary (6500).

In some embodiments, a single stopping criterion used. In other embodiments, more than one stopping criteria are used. In one embodiment one stopping criterion is a value reflecting the mean separation distance between genome sequence segments within the target genome sequence(s). For example, a mean distance between genome sequence segments is a whole or fractional number less or equal to about 500, 600, 700, 900, or 1000 nucleobases or any whole or fractional number therebetween. In other embodiments, the stopping criterion is the mean distance between genome sequence segments within the target genome sequence(s) or a value above or below the mean distance between genome sequence segments within the target genome sequence(s).

In other embodiments, a stopping criterion is the maximum distance between any two of the selected genome sequence segments within the target genome sequence(s). For example, an appropriate maximum distance between any two genome sequence segments might be less than or equal to about 5,000, 6,000, 7,000, 8,000, 9,000 or 10,000 nucleobases or any number therebetween.

In some embodiments, after the stopping criterion or criteria have been met and the computer executable instructions are complete, the in silico method produces an output report comprising a list of genome sequence segments. The report may be a print-out or a display on a graphical interface or any other means for displaying the results of the selection process. The in silico method may also provide a means for designing primers that hybridize to the genome sequence segments.

### Example 3

### Selection of Primer Sets for Targeted Whole Genome Amplification

In a first example for targeted whole genome amplification, Bacillus anthracis Ames was chosen as a single target genome. The set of background genomes included the genomes of: Homo sapiens, Gallus gallus, Guillardia theta, Oryza sativa, Arabidopsis thaliana, Yarrowia lipolytica, Saccharomyces cerevisiae, Debaryomyces hansenii, Kluyveromyces lactis, Schizosaccharomyces pom, Aspergillus fumigatus, Cryptococcus neoformans, Encephalitozoon cuniculi, Eremothecium gossypii, Candida glabrata, Apis mellifera, Drosophila melanogaster, Tribolium castaneum, Anopheles gambiae, and Caenorhabditis elegans. These background genomes were chosen because they would be expected to be present in a typical soil sample handled by a human.

Unique genome sequence segments 7 to 12 nucleobases in length were identified. Frequency of occurrence and selectivity ratio values were determined. As a result, 200 genome sequence segments were identified. In most cases, the primers designed to hybridize with 100% complementarity to its corresponding genome sequence segment. In a few other cases, degenerate primers were prepared. The degenerate bases of the primers occur at positions complementary to positions having ambiguity within the target Bacillus anthracis genome or complementary to positions known or thought to be susceptible to single nucleotide polymorphisms. The 200 primers (Table 3) designed to hybridize to the genome sequence segments were found to have a combined total of 12822 hybridization sites. The mean separation distance of the genome sequence segments and the primers hybridizing thereto was found to be 815 nucleobases in length. The maximum distance between the genome sequence segments and the primers hybridizing thereto was found to be 5420 nucleobases in length. The mean “frequency bias” of hybridization of a primer to the target genome relative to the background genomes was calculated to be 3.3 1, indicating that the average primer hybridizes at 3.31 different positions on the target genome sequence for each single position it hybridizes to a background genome sequence.

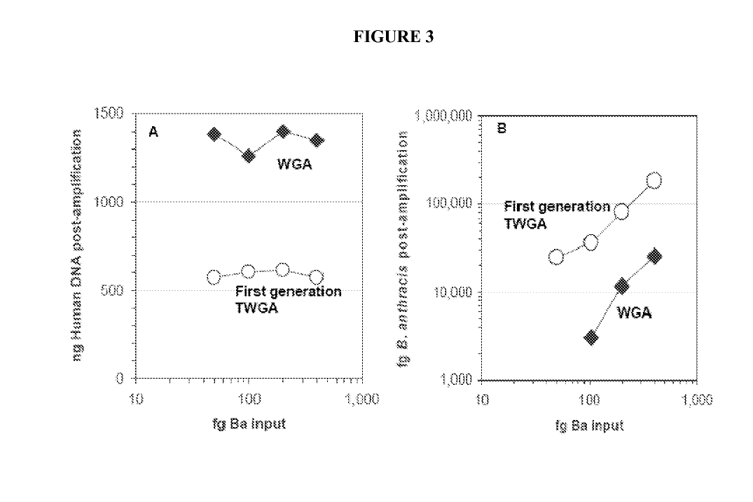

In an experiment designed to test the efficiency of the targeted whole genome amplification reaction vs. traditional whole genome amplification, reactions were carried out using 50, 100, 200, and 400 femtograms of Bacillus anthracis Sterne genomic DNA in the presence of 100 nanograms of human genomic DNA. Amplified quantities of DNA were determined and it was found that the targeted whole genome amplification reactions resulted in much greater specificity toward amplification of Bacillus anthracis Sterne genomic DNA than human genomic DNA. FIG. 3A indicates that ordinary whole genome amplification using random primers 6 nucleobases in length under the conditions listed above results in production of larger quantities of human genomic DNA, as would be expected. FIG. 3B, on the other hand indicates that the 200 primers described above selectively amplify the Bacillus anthracis Sterne genomic DNA relative to the human DNA, even though the quantity of Bacillus anthracis Sterne genomic DNA was much lower than the human genomic DNA.

A second experiment was conducted where additional target genomes were selected for the primer design process. The group of total target genomes included the genomes of the following potential biowarfare agents: Bacillus anthracis, Francisella tularensis, Yersinia pestis, Brucella sp., Burkholderia mallei, Rickettsia prowazekii, and Escherichia coli 0157. The group of background genomes was expanded. An exact match BLAST was used to determine the frequency of occurrence of genome sequence segments in the background genomes. A larger number of genome sequence segments was analyzed and query 3 (FIG. 2—6000) was automated. The 200 primers designed in the first experiment are shown in Table 3 and the 191 primers designed in the second experiment are shown in Table 4. In Tables 3 and 4, an asterisk (*) indicates a phosphorothioate linkage and degenerate nucleobases codes are as follows: r=a or g; k=g or t; s=g or c; y=c or t; m=a or c, and w=a or t.

TABLE 3
First Generation Targeted Whole Genome
Amplification Primer Set

| Sequence | SEQ ID NO: |
|---|---|
| aaaaaagc*g*g | 203 |
| aaaacg*c*t | 204 |
| aaaagaagtt*a*t | 205 |
| aaaaggc*g*g | 206 |
| aaaccgc*c*a | 207 |
| aaaccgt*a*t | 208 |
| aaaccgt*t*a | 209 |
| aaagaagaag*t*t | 210 |
| aaagaagctt*t*a | 211 |
| aaagaagtat*t*a | 212 |
| aaagccg*a*t | 213 |
| aaagcgtggg*g*a | 214 |
| aaagtagaag*a*a | 215 |
| aaataacg*a*t | 216 |
| aaatacg*c*t | 217 |
| aaatcattaa*a*g | 218 |
| aaattag*c*g | 219 |
| aaccgcc*t*t | 220 |
| aacgat*t*g | 221 |
| aacgata*t*t | 222 |
| aacgctt*c*w | 223 |
| aacgtga*a*c | 224 |
| aacttctttt*t*c | 225 |
| aagaaac*g*c | 226 |
| aagarttaaa*a*g | 227 |
| aagataaaga*t*g | 228 |
| aagatgtaaa*a*g | 229 |
| aagcatctaa*g*c | 230 |
| aagcgat*c*a | 231 |
| aagcggt*t*c | 232 |
| aagtaac*g*a | 233 |
| aataacg*c*a | 234 |
| aatattggac*a*a | 235 |
| aatcattaat*a*t | 236 |
| aatccag*c*g | 237 |
| aatcgcc*c*a | 238 |
| aatcgta*t*c | 239 |
| aatcgtt*a*a | 240 |
| aatcgtt*g*c | 241 |
| aatctggtgg*t*a | 242 |
| aatgcg*g*t | 243 |
| aattaa*c*g | 244 |
| aatttcatct*a*a | 245 |
| accgata*a*t | 246 |
| accgcat*c*a | 247 |
| acgaatg*a*t | 248 |
| acgatgt*t*g | 249 |
| acggtta*t*c | 250 |
| acggttt*t*a | 251 |
| acgrtaa*a*a | 252 |
| acgttt*a*t | 253 |
| acttttttat*c*t | 254 |
| agaattatta*a*a | 255 |
| agataaa*c*g | 256 |
| agatgaaaat*g*g | 257 |
| agcaatc*g*c | 258 |
| agcagttgca*g*c | 259 |
| agcgcaa*t*c | 260 |
| agcttgt*t*g | 261 |
| agttgat*c*g | 262 |
| ataaaaaaag*c*g | 263 |
| ataaaaaagg*t*a | 264 |
| ataaagaaga*t*g | 265 |
| ataaagatat*t*a | 266 |
| ataacga*a*g | 267 |
| ataactaata*a*a | 268 |
| ataatagaag*a*a | 269 |
| ataccatttt*t*a | 270 |
| atacgat*a*a | 271 |
| atagatgaaa*a*t | 272 |
| atagcga*t*a | 273 |
| atatcgt*a*a | 274 |
| atatcttttt*c*a | 275 |
| atattaaa*g*c | 276 |
| atattgaaga*a*g | 277 |
| atattgat*a*c | 278 |
| atcagct*a*c | 279 |
| atcatgc*c*g | 280 |
| atcgcac*c*g | 281 |
| atcgcctt*c*a | 282 |
| atcgtaa*t*a | 283 |
| atcgtga*a*g | 284 |
| atcgtta*a*a | 285 |
| atcttca*c*g | 286 |
| atcttcttta*a*t | 287 |
| attaata*c*c | 288 |
| attacaa*c*g | 289 |
| attacaac*a*a | 290 |
| attacc*g*c | 291 |
| attagaagaa*a*t | 292 |
| attatc*g*g | 293 |
| attatcg*t*a | 294 |
| attcatc*g*g | 295 |
| attgatat*t*a | 296 |
| attgatataa*a*t | 297 |
| attgatgaa*g*c | 298 |
| attgatgatt*t*a | 299 |
| attgcagc*a*a | 300 |
| atttagataa*a*t | 301 |
| atttagatga*a*g | 302 |
| atttatca*g*c | 303 |
| atttattatt*a*g | 304 |
| atttctttat*c*a | 305 |
| caatcgg*t*g | 306 |
| caatcgy*t*a | 307 |
| cacctttttt*a*a | 308 |
| cagcgat*t*a | 309 |
| cagcttttt*t*a | 310 |
| catcgct*t*c | 311 |
| catctaaaat*a*a | 312 |
| catcttc*c*g | 313 |
| ccaatcg*g*c | 314 |
| cccgctt*c*a | 315 |
| ccggtaa*t*a | 316 |
| cgataat*g*a | 317 |
| cgattaa*a*g | 318 |
| cgattg*c*g | 319 |
| cgcctct*t*c | 320 |
| cgctaaa*t*a | 321 |
| cgcttta*t*a | 322 |
| cggcgcgctg*a*a | 323 |
| cggtatt*g*a | 324 |
| cgtaaag*a*a | 325 |
| cgtaaat*a*c | 326 |
| cgtgatc*a*a | 327 |
| cgtttat*t*a | 328 |
| cgwtaat*a*a | 329 |
| ctaattcttc*t*a | 330 |
| ctactttttc*c*a | 331 |
| ctgtagaaga*a*g | 332 |
| ctgttttaga*a*g | 333 |
| cttcacg*a*a | 334 |
| cttcatca*a*c | 335 |
| cttcatctaa*t*a | 336 |
| cttcttctaa*a*a | 337 |
| cttcttcttt*a*a | 338 |
| cttctttc*g*c | 339 |
| ctttagaaaa*t*a | 340 |
| ctttatataa*a*r | 341 |
| ctttatcaat*a*a | 342 |
| ctttcgct*t*c | 343 |
| cttttatata*a*a | 344 |
| ctttttcwtc*t*a | 345 |
| gaaaaaggat*t*a | 346 |
| gaaacga*t*c | 347 |
| gaaacgt*t*a | 348 |
| gaaattgctg*a*c | 349 |
| gaagaagyga*a*a | 350 |
| gaagatgaaa*a*a | 351 |
| gaagatttat*t*a | 352 |
| gaagtattaa*a*a | 353 |
| gaatatgaag*a*a | 354 |
| gatattgata*a*a | 355 |
| gatgaagata*a*a | 356 |
| gatttattat*t*a | 357 |
| gatttcacga*a*a | 358 |
| gcaata*a*c | 359 |
| gccttt*a*c | 360 |
| gcgaaag*a*a | 361 |
| gcgattt*t*a | 362 |
| gcggtat*t*a | 363 |
| gcgttaa*t*a | 364 |
| gcgttta*a*a | 365 |
| gcgtttt*g*a | 366 |
| gckgatt*t*a | 367 |
| gctaaaaaag*a*a | 368 |
| gctattttat*t*a | 369 |
| gctcgcgcga*c*a | 370 |
| gcttctttta*t*a | 371 |
| gctttttcat*c*a | 372 |
| ggcatt*a*c | 373 |
| ggcggta*a*a | 374 |
| ggttgaa*a*c | 375 |
| ggttta*a*c | 376 |
| gtaaaac*g*a | 377 |
| gtaaagcttt*c*a | 378 |
| gtgacga*a*a | 379 |
| gttatcg*c*a | 380 |
| gttgttttac*c*a | 381 |
| sttccgc*a*a | 382 |
| taaaatgggt*g*a | 383 |
| taaagcaatt*a*a | 384 |
| taaatcatct*a*a | 385 |
| taacgaa*g*a | 386 |
| taactcttct*a*a | 387 |
| taatgctt*c*a | 388 |
| tacatcat*c*a | 389 |
| tatcatc*g*a | 390 |
| tatcattaat*a*a | 391 |
| tatcctcttc*c*a | 392 |
| tcttctaata*a*a | 393 |
| tcttctaatt*c*a | 394 |
| tcttcttcta*a*a | 395 |
| tcttttttta*c*a | 396 |
| tgacgat*a*a | 397 |
| tgatgcg*a*a | 398 |
| tgcttctttt*a*a | 399 |
| ttagatgaag*a*a | 400 |
| ttagctaaag*a*a | 401 |
| ttattagaag*a*a | 402 |

TABLE 4
Second Generation Targeted Whole Genome
Amplification Primer Set

| Sequence | SEQ ID NO: |
|---|---|
| aaaacaat*t*g | 403 |
| aaaacgtt*t*a | 404 |
| aaaagaat*t*a | 405 |
| aaaaggta*t*t | 406 |
| aaaaggtg*a*a | 407 |
| aaataacg*a*t | 216 |
| aaatcgttga*t*a | 409 |
| aaatggtga*a*g | 410 |
| aacaccaa*t*t | 411 |
| aacgaaag*a*t | 412 |
| aacgaaagaa*g*a | 413 |
| aacgaat*a*a | 414 |
| aagaagcga*a*g | 415 |
| aagaagtaaa*a*g | 416 |
| aagcg*g*a | 417 |
| aatcgc*t*a | 418 |
| aatcgcaa*t*t | 419 |
| aatcgcygat*a*t | 420 |
| aatcgttt*c*a | 421 |
| acaacga*t*t | 422 |
| accgataa*t*a | 423 |
| acgaagc*a*a | 424 |
| agaagcgat*g*a | 425 |
| agcgaaaga*a*g | 426 |
| atacga*t*g | 427 |
| atacgg*a*a | 428 |
| atataaaa*g*a | 429 |
| atatg*c*g | 430 |
| atattatc*g*t | 431 |
| atcarcgatt*t*t | 432 |
| atcata*c*g | 433 |
| atccgt*t*a | 434 |
| atgaag*c*g | 435 |
| atgtaac*g*a | 436 |
| attaaagat*g*g | 437 |
| attaac*g*c | 438 |
| attacaaa*a*g | 439 |
| attacgat*a*a | 440 |
| attacgt*t*a | 441 |
| attacttg*t*a | 442 |
| attatatg*a*a | 443 |
| attattat*c*g | 444 |
| attgaaaaag*c*a | 445 |
| attgaaac*g*a | 446 |
| attgcttc*t*t | 447 |
| attgtcg*t*t | 448 |
| atttatcg*t*a | 449 |
| caacttct*t*t | 450 |
| caatcgt*a*t | 451 |
| caattaat*a*c | 452 |
| caattgga*a*t | 453 |
| caccaatt*a*c | 454 |
| caccaatt*g*t | 455 |
| cacctttta*c*a | 456 |
| catacg*a*a | 457 |
| catataa*c*g | 458 |
| catcaattg*t*t | 459 |
| ccgct*t*t | 460 |
| cgacttaccg*a*c | 461 |
| cgata*a*c | 462 |
| cgataaag*a*a | 463 |
| cgatataat*t*t | 464 |
| cgatg*t*a | 465 |
| cgattga*a*g | 466 |
| cgatttttc*a*a | 467 |
| cgcaa*t*a | 468 |
| cgcttttta*t*t | 469 |
| cggat*a*t | 470 |
| cggtaa*a*t | 471 |
| cggttta*a*t | 472 |
| cgtaat*a*t | 473 |
| cgtata*a*c | 474 |
| cgttaat*t*g | 475 |
| cgttatg*a*a | 476 |
| ctatcg*t*a | 477 |
| ctgattaaag*t*t | 478 |
| cttccata*a*t | 479 |
| cttcgt*a*a | 480 |
| cttctata*t*a | 481 |
| cttctgca*a*t | 482 |
| cttcttca*c*g | 483 |
| cttcttcttt*c*g | 484 |
| cttcttta*a*t | 485 |
| cttctttc*g*c | 339 |
| cttctttcg*g*a | 487 |
| ctttcgct*t*t | 488 |
| ctttcgcttc*t*t | 489 |
| cttttaattc*t*t | 490 |
| cttttgtaa*t*a | 491 |
| ctttttcg*t*a | 492 |
| cttttttc*a*t | 493 |
| ctttttya*t*c | 494 |
| gaaacgat*t*g | 495 |
| gaagaagcga*a*a | 496 |
| gaagaagt*a*a | 497 |
| gaagaagta*g*c | 498 |
| gatacgaa*a*g | 499 |
| gatgaatt*a*g | 500 |
| gatta*c*g | 501 |
| gattaaagtt*t*c | 502 |
| gcaattgaaa*a*a | 503 |
| gcaattgt*a*t | 504 |
| gcaattgt*t*g | 505 |
| gcgaaagaa*g*c | 506 |
| gcgtaa*t*a | 507 |
| gctacttt*a*t | 508 |
| gcttcttt*c*g | 509 |
| gcttttttta*t*t | 510 |
| gtattaaaa*g*a | 511 |
| gttaattg*a*a | 512 |
| gttcg*t*a | 513 |
| gttgc*g*a | 514 |
| taaagataa*t*g | 515 |
| taaagcg*t*t | 516 |
| taaagtgaaa*c*t | 517 |
| taaatcttc*t*a | 518 |
| taacagaa*g*a | 519 |
| taacgaaaga*a*g | 520 |
| taacgga*a*a | 521 |
| taactcttc*t*t | 522 |
| taatam*c*g | 523 |
| taatcg*y*a | 524 |
| taatgaag*a*a | 525 |
| taattgct*t*c | 526 |
| tacaattt*c*a | 527 |
| taccgt*t*a | 528 |
| tacgaaaga*a*g | 529 |
| tacgaatg*a*t | 530 |
| tactcg*t*t | 531 |
| tagaagaa*g*t | 532 |
| tagaagaag*c*g | 533 |
| tagaagc*g*a | 534 |
| tatatcgact*t*a | 535 |
| tatatcrgcg*a*t | 536 |
| tatcggcgat*t*t | 537 |
| tatgtaa*c*g | 538 |
| tattag*c*g | 539 |
| tattcg*c*t | 540 |
| tattgatg*a*a | 541 |
| tawtacga*a*a | 542 |
| tcaattgc*a*a | 543 |
| tcaattgct*t*c | 544 |
| tcattac*g*a | 545 |
| tccaattg*a*a | 546 |
| tccgaaag*a*a | 547 |
| tccgct*a*a | 548 |
| tccgt*a*t | 549 |
| tcctgtta*c*a | 550 |
| tcgca*t*a | 551 |
| tcgcttta*t*t | 552 |
| tcgtat*t*g | 553 |
| tcgttaca*a*t | 554 |
| tctacaat*t*a | 555 |
| tctactaa*t*t | 556 |
| tcttcaat*a*t | 557 |
| tcttctaa*c*g | 558 |
| tctttata*t*g | 559 |
| tctttatat*t*c | 560 |
| tctttcgc*t*a | 561 |
| tcttttttc*g*c | 562 |
| tgaaaaag*c*g | 563 |
| tgaaacaat*t*g | 564 |
| tgaaacga*a*t | 565 |
| tgaagcga*t*t | 566 |
| tgcaa*c*g | 567 |
| tgcgaaaga*a*a | 568 |
| tgcttcttc*t*a | 569 |
| tgtaaaag*g*t | 570 |
| tgtcggtaag*t*c | 571 |
| tgttctttc*g*t | 572 |
| ttaacgaaa*g*a | 573 |
| ttaacgg*a*a | 574 |
| ttacgaaa*g*a | 575 |
| ttagaaga*t*g | 576 |
| ttattatc*g*g | 577 |
| ttcaata*c*g | 578 |
| ttcacgaa*t*a | 579 |
| ttccgt*a*a | 580 |
| ttcgtaaa*t*t | 581 |
| ttcttta*c*g | 582 |
| ttctttcg*c*a | 583 |
| ttctttcgtt*a*a | 584 |
| ttctttta*t*a | 585 |
| ttgcaatt*g*c | 586 |
| ttgtaatt*g*g | 587 |
| ttgtcggta*a*g | 588 |
| tttattaga*t*g | 589 |
| tttcgtat*a*t | 590 |
| tttcgtta*t*a | 591 |
| tttwtcgt*a*a | 592 |
| twacgat*t*g | 593 |

Table 5 shows a comparison of statistics obtained from the first and second experiments. The statistics indicate the likelihood that more selective and efficient priming of the target Bacillus anthracis genome would be expected under the conditions of the second generation proof-of-concept experiment.

TABLE 5
Statistical Comparison of First and Second Experiments

| | First | Second |
|---|---|---|
| | Generation | Generation |
| Statistic | Experiment | Experiment |
| Total Frequency of Occurrence of all | 12822 | 25822 |
| Selected Genome Sequence Segments | | |
| Mean Separation Distance Between | 815 | 404 |
| Selected Genome Sequence Segments | | |
| Maximum Separation Distance Between | 5420 | 3477 |
| Selected Genome Sequence Segments | | |
| Average Frequency Bias to Target | 3.31 | 4.67 |
| Genome Over Background Genomes | | |

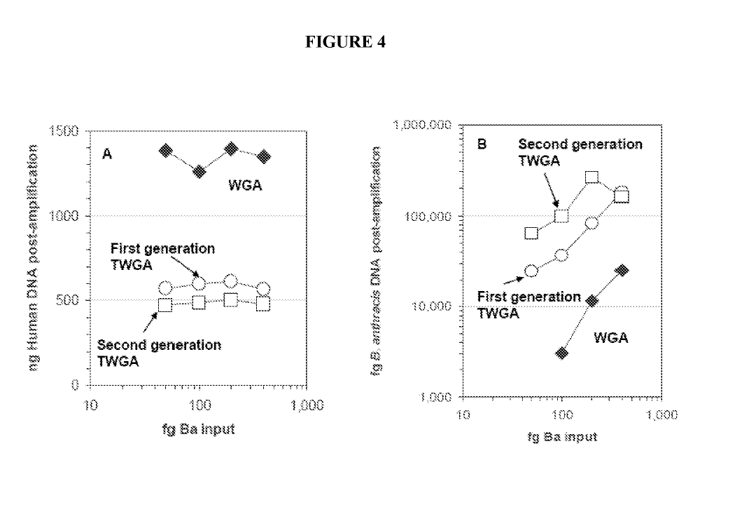

The results of the second generation experiment are shown in FIGS. 4A and 4B. It is readily apparent that the modifications to the selection process added in the second experiment result in a more efficient targeted whole genome amplification reaction which is biased toward amplification of the Bacillus anthracis target genome. The primers of Table 4 produce less human DNA and more Bacillus anthracis DNA than the traditional whole genome amplification (WGA) and the first generation primer set (Table 3). Furthermore, the frequency bias was found to be even higher for the remaining target genomes as shown in Table 6.

TABLE 6
Statistical Comparison of Genome Sequence Segments
for the Target Genomes of the Second Experiment

| | Total | | Maximum | |
|---|---|---|---|---|
| | Frequency of | Mean | Distance | Mean |
| | Occurrence | Separation | Between | Frequency |
| Target Genome | of Segments | Distance | Segments | Bias |
| Bacillus anthracis | 25822 | 404.84 | 3477 | 4.67 |
| Rickettsia prowazekii | 5606 | 396.41 | 2265 | 5.44 |
| Escherichia coli | 23501 | 467.89 | 4822 | 22.70 |
| Yersinia pestis | 18597 | 500.43 | 4616 | 35.69 |
| Brucella sp. | 13442 | 490.10 | 3527 | 41.96 |
| Francisella tularensis | 7925 | 477.56 | 3179 | 50.08 |
| Burkholderia mallei | 25218 | 462.73 | 4062 | 291.13 |

### Example 4

### Targeted Whole Genome Amplification Protocol

The targeted whole genome amplification reaction mixture consisted of: 5 microliters of template DNA, and 0.04025 M TRIS HCl, 0.00975 M TRIS base, 0.012 M MgCl₂, 0.01 M (NH₄)₂SO₄, 0.8 M betaine, 0.8 M trehalose, 25 mM of each deoxynucleotide triphosphate (Bioline, Randolph, Mass., U.S.A), 0.004 M dithiothreitol, 0.05 mM of primers of the selected primer set, and 0.5 units of Phi29 polymerase enzyme per microliter of reaction mixture. The thermal cycling conditions for the amplification reaction were as follows: 1. 30° C. for 4 minutes 2. 15° C. for 15 seconds 3. repeat steps 1 and 2.times.150 4. hold at 95° C. for 10 minutes 5. hold at 4° C. until ready for analysis.

### Example 5

### Targeted Whole Genome Amplification of Sepsis-Causing Microorganisms

This example is directed toward design of a kit for targeted whole genome amplification of organisms which are known to cause sepsis. A collection of target genomes is assembled, comprising the genomes of the following microorganisms known to cause bloodstream infections: Escherichia coli, Klebsiella pneumoniae, Klebsiella oxytoca, Serratia marcescens, Enterobacter cloacae, Enterobacter aerogenes, Proteus mirabilis, Pseudomonas aeruginosa, Acinetobacter baumannii, Stenotrophomonas maltophilia, Staphylococcus aureus, Staphylococcus epidermidis, Staphylococcus haemolyticus, Streptococcus pneumoniae, Streptococcus pyogenes, Streptococcus agalactiae, Streptococcus mitis, Enterococcus faecium, Enterococcus faecalis, Candida albicans, Candida tropicalis, Candida parapsilosis, Candida krusei, Candida glabrata and Aspergillus fumigatus. Because the healthy human bloodstream generally does not contain microorganisms or parasites, only the human genome is chosen as a single background genome. Alternatively, if a human was known to be infected with a virus such as HIV or HCV for example, the genomes of HIV or HCV could be included as background genomes during the primer design process. Genomes commonly found in the human bloodstream are considered background genomes.

The target and background genomes are obtained from a genomics database such as GenBank. The target genomes are scanned by a computer program to identify all unique genome sequence segments between 5 and 13 nucleobases in length. The computer program further determines and records the frequency of occurrence of each of the unique genome sequence segments within each of the target genomes.

The human genome is then scanned to determine the frequency of occurrence of the genome sequence segments. Optionally, the entire list of genome sequence segments is reduced by removing genome sequence segments that have low frequencies of occurrence by choosing an arbitrary frequency of occurrence threshold criterion such as, for example, the mean frequency of occurrence or any frequency of occurrence 25% above or below the mean frequency of occurrence or any whole or fractional percentage therebetween. For example, if the mean frequency of occurrence is 100, 25% above 100 equals 125 and 25% below 100 equals 75 and the frequency of occurrence threshold criterion may be any whole or fractional number between about 75 and about 125. When this step is complete, a subset of the original list of unique genome sequence segments remains. At this point, the subset of genome sequence subsets is analyzed by the computer program to determine the frequency of occurrence of each of the genome sequence segments within the human genome. Upon completion of this step, the genome sequence segments of the subset are associated with the following data; the frequency of occurrence within each of the target genomes and the frequency of occurrence within the human genome. A value indicating the total target frequency of occurrence is calculated by adding the frequency of occurrence of the genome sequence segments in each of the target genomes. The selectivity ratio is calculated by the computer program for the genome sequence segments of the subset by dividing the total target frequency of occurrence by the background frequency of occurrence. When the series of selectivity ratio calculations are complete, the genome sequence segments are ranked by their selectivity ratio values such that the highest selectivity ratio receives the highest rank. The ranked genome sequence segments are then subjected to the process described Example 2 and illustrated in FIG. 2.

The process of Example 2 and FIG. 2 ends when the pre-determined quantity of 200 genome sequence segments is reached and when the stopping criteria are met. The stopping criteria are the following: the mean distance between the selected genome sequence segments on the target genomes is less than 500 nucleobases and the maximum distance between the selected genome sequence segments on the target genomes is less than 5000 nucleobases. These values are calculated by the computer program from the known coordinates of the target genomes and the selected genome sequence segments.

The primer design step begins after completion of the selection process of the genome sequence segments. The genome sequence segments represent primer hybridization sites and a primer is designed to bind to each of the selected genome sequence segments. For an initial round of primer design and testing, primers are designed to be 100% complementary to each of the selected genome sequence segments. Optionally, the primers can be subjected to an in silico analysis to determine if they unfavorable characteristics. Unfavorable characteristics may include poor affinity (as measured by melting temperature) for their corresponding target genome sequence segment, primer dimer formation, or presence of secondary structure. Upon identification of unfavorable characteristics in a given primer, the primer is redesigned by alteration of length or by incorporation of modified nucleobases.

Once primer design (and redesign if necessary) is complete, the primers are synthesized and subjected to in vitro testing by amplification of the target genomes in the presence of human DNA (representing the background human genome) to determine the amplification efficiency and bias toward the target genomes. Analyses such as those shown in FIGS. 3 and 4 are useful for determining these measures. In addition, analyses of statistics such as those shown in Table 6 are useful for obtaining an estimation of bias toward the target genomes relative to the background human genome.

When the primer design and testing is complete, kits are assembled. The kits contain the primers, deoxynucleotide triphosphates, a processive polymerase, buffers and additives useful for improving the yield of amplified genomes. These kits are used to amplify genomic DNA of sepsis-causing organisms from blood samples of individuals exhibiting symptoms of sepsis. The amplified DNA is then available for further testing for the purpose of genotyping. Such tests include real-time PCR, microarray analysis and triangulation genotyping analysis by mass spectrometry of bioagent identifying amplicons as described herein (Examples 6-12). Additionally, genotyping of sepsis-causing organisms is useful in determining an appropriate course of treatment with antibiotics and alerting authorities of the presence of potentially drug-resistant strains of sepsis-causing organisms. Such genotyping analyses can be developed using methods described herein as well as those disclosed in commonly owned U.S. application Ser. No. 11/409,535 which is incorporated herein by reference in entirety.

### Example 6

### Design and Validation of Primer Pairs that Define Bioagent Identifying Amplicons for Identification of Bacteria

For design of primers that define bacterial bioagent identifying amplicons, a series of bacterial genome segment sequences are obtained, aligned and scanned for regions where pairs of PCR primers would amplify products of about 39 to about 200 nucleotides in length and distinguish subgroups and/or individual strains from each other by their molecular masses or base compositions. A typical process shown in FIG. 8 is employed for this type of analysis. A database of expected base compositions for each primer region is generated using an in silico PCR search algorithm, such as (ePCR). An existing RNA structure search algorithm (Macke et al., Nucl. Acids Res., 2001, 29, 4724-4735, which is incorporated herein by reference in its entirety) has been modified to include PCR parameters such as hybridization conditions, mismatches, and thermodynamic calculations (Santa Lucia, Proc. Natl. Acad. Sci. U.S.A., 1998, 95, 1460-1465, which is incorporated herein by reference in its entirety). This also provides information on primer specificity of the selected primer pairs. An example of a collection of such primer pairs is disclosed in U.S. application Ser. No. 11/409,535 which is incorporated herein by reference in entirety.

### Example 7

### Sample Preparation and PCR

Genomic DNA id prepared from samples using the DNeasy Tissue Kit (Qiagen, Valencia, Calif.) according to the manufacturer's protocols.

PCR reactions are assembled in 50 μL reaction volumes in a 96-well microtiter plate format using a Packard MPII liquid handling robotic platform and M. J. Dyad thermocyclers (MJ research, Waltham, Mass.) or Eppendorf Mastercycler thermocyclers (Eppendorf, Westbury, N.Y.). The PCR reaction mixture includes of 4 units of Amplitaq Gold, 1× buffer II (Applied Biosystems, Foster City, Calif.), 1.5 mM MgCl₂, 0.4 M betaine, 800 μM dNTP mixture and 250 nM of each primer. The following typical PCR conditions are used: 95° C. for 10 min followed by 8 cycles of 95° C. for 30 seconds, 48° C. for 30 seconds, and 72° C. 30 seconds with the 48° C. annealing temperature increasing 0.9° C. with each of the eight cycles, The PCR reaction is then continued for 37 additional cycles of 95° C. for 15 seconds, 56° C. for 20 seconds, and 72° C. 20 seconds.

### Example 8

### Purification of PCR Products for Mass Spectrometry with Ion Exchange Resin-Magnetic Beads

For solution capture of nucleic acids with ion exchange resin linked to magnetic beads, 25 μl of a 2.5 mg/mL suspension of BioClone amine-terminated superparamagnetic beads is added to 25 to 50 μl of a PCR (or RT-PCR) reaction containing approximately 10 pM of a typical PCR amplification product. The above suspension is mixed for approximately 5 minutes by vortexing or pipetting, after which the liquid is removed after using a magnetic separator. The beads containing bound PCR amplification product are then washed three times with 50 mM ammonium bicarbonate/50% MeOH or 100 mM ammonium bicarbonate/50% MeOH, followed by three more washes with 50% MeOH. The bound PCR amplification product is eluted with a solution of 25 mM piperidine, 25 mM imidazole, 35% MeOH which includes peptide calibration standards.

### Example 9

### Mass Spectrometry and Base Composition Analysis

The ESI-FTICR mass spectrometer is based on a Bruker Daltonics (Billerica, Mass.) Apex II 70e electrospray ionization Fourier transform ion cyclotron resonance mass spectrometer that employs an actively shielded 7 Tesla superconducting magnet. The active shielding constrains the majority of the fringing magnetic field from the superconducting magnet to a relatively small volume. Thus, components that might be adversely affected by stray magnetic fields, such as CRT monitors, robotic components, and other electronics, can operate in close proximity to the FTICR spectrometer. All aspects of pulse sequence control and data acquisition were performed on a 600 MHz Pentium II data station running Bruker's Xmass software under Windows NT 4.0 operating system. Sample aliquots, typically 15 μl, are extracted directly from 96-well microtiter plates using a CTC HTS PAL autosampler (LEAP Technologies, Carrboro, N.C.) triggered by the FTICR data station. Samples are injected directly into a 10 μl sample loop integrated with a fluidics handling system that supplies the 100 μl/hr flow rate to the ESI source. Ions are formed via electrospray ionization in a modified Analytica (Branford, Conn.) source employing an off axis, grounded electrospray probe positioned approximately 1.5 cm from the metallized terminus of a glass desolvation capillary. The atmospheric pressure end of the glass capillary is biased at 6000 V relative to the ESI needle during data acquisition. A counter-current flow of dry N₂ is employed to assist in the desolvation process. Ions are accumulated in an external ion reservoir comprised of an rf-only hexapole, a skimmer cone, and an auxiliary gate electrode, prior to injection into the trapped ion cell where they are mass analyzed. Ionization duty cycles greater than 99% are achieved by simultaneously accumulating ions in the external ion reservoir during ion detection. Each detection event includes 1M data points digitized over 2.3 s. To improve the signal-to-noise ratio (S/N), 32 scans are co-added for a total data acquisition time of 74 s.

The ESI-TOF mass spectrometer is based on a Bruker Daltonics MicroTOF™. Ions from the ESI source undergo orthogonal ion extraction and are focused in a reflectron prior to detection. The TOF and FTICR are equipped with the same automated sample handling and fluidics described above. Ions are formed in the standard MicroTOF™ ESI source that is equipped with the same off-axis sprayer and glass capillary as the FTICR ESI source. Consequently, source conditions were the same as those described above. External ion accumulation is also employed to improve ionization duty cycle during data acquisition. Each detection event on the TOF includes 75,000 data points digitized over 75 μs.

The sample delivery scheme allows sample aliquots to be rapidly injected into the electrospray source at high flow rate and subsequently be electrosprayed at a much lower flow rate for improved ESI sensitivity. Prior to injecting a sample, a bolus of buffer is injected at a high flow rate to rinse the transfer line and spray needle to avoid sample contamination/carryover. Following the rinse step, the autosampler injects the next sample and the flow rate is switched to low flow. Following a brief equilibration delay, data acquisition commenced. As spectra are co-added, the autosampler continued rinsing the syringe and picking up buffer to rinse the injector and sample transfer line. In general, two syringe rinses and one injector rinse are required to minimize sample carryover. During a routine screening protocol a new sample mixture is injected every 106 seconds. More recently a fast wash station for the syringe needle has been implemented which, when combined with shorter acquisition times, facilitates the acquisition of mass spectra at a rate of just under one spectrum/minute.

Raw mass spectra are post-calibrated with an internal mass standard and deconvoluted to monoisotopic molecular masses. Unambiguous base compositions are derived from the exact mass measurements of the complementary single-stranded oligonucleotides. Quantitative results are obtained by comparing the peak heights with an internal PCR calibration standard present in every PCR well at 500 molecules per well. Calibration methods are commonly owned and disclosed in PCT Publication Number WO 2005/098047 which is incorporated herein by reference in entirety.

### Example 10

### De Novo Determination of Base Composition of Amplification Products Using Molecular Mass Modified Deoxynucleotide Triphosphates

Because the molecular masses of the four natural nucleobases have a relatively narrow molecular mass range (A=313.058, G=329.052, C=289.046, T=304.046—See Table 7), a persistent source of ambiguity in assignment of base composition can occur as follows: two nucleic acid strands having different base composition may have a difference of about 1 Da when the base composition difference between the two strands is G⇄A (−15.994) combined with C⇄T (+15.000). For example, one 99-mer nucleic acid strand having a base composition of A₂₇G₃₀C₂₁T₂₁ has a theoretical molecular mass of 30779.058 while another 99-mer nucleic acid strand having a base composition of A₂₆G₃₁C₂₂T₂₀ has a theoretical molecular mass of 30780.052. A 1 Da difference in molecular mass may be within the experimental error of a molecular mass measurement and thus, the relatively narrow molecular mass range of the four natural nucleobases imposes an uncertainty factor.

The methods provide for a means for removing this theoretical 1 Da uncertainty factor through amplification of a nucleic acid with one mass-tagged nucleobase and three natural nucleobases. The term “nucleobase” as used herein is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

Addition of significant mass to one of the 4 nucleobases (dNTPs) in an amplification reaction, or in the primers themselves, will result in a significant difference in mass of the resulting amplification product (significantly greater than 1 Da) arising from ambiguities arising from the G⇄A combined with C⇄T event (Table 7). Thus, the same the G⇄A (−15.994) event combined with 5-Iodo-C⇄T (−110.900) event would result in a molecular mass difference of 126.894. If the molecular mass of the base composition A₂₇G₃₀-5-Iodo-C₂₁T₂₁ (33422.958) is compared with A₂₆G₃₁-5-Iodo-C₂₂T₂₀, (33549.852) the theoretical molecular mass difference is +126.894. The experimental error of a molecular mass measurement is not significant with regard to this molecular mass difference. Furthermore, the only base composition consistent with a measured molecular mass of the 99-mer nucleic acid is A₂₇G₃₀-5-Iodo-C₂₁T₂₁. In contrast, the analogous amplification without the mass tag has 18 possible base compositions.

TABLE 7
Molecular Masses of Natural Nucleobases and the
Mass-Modified Nucleobase 5-Iodo-C and Molecular
Mass Differences Resulting from Transitions

| Nucleobase | Molecular Mass | Transition | Δ Molecular Mass |
|---|---|---|---|
| A | 313.058 | A-->T | −9.012 |
| A | 313.058 | A-->C | −24.012 |
| A | 313.058 | A-->5-Iodo-C | 101.888 |
| A | 313.058 | A-->G | 15.994 |
| T | 304.046 | T-->A | 9.012 |
| T | 304.046 | T-->C | −15.000 |
| T | 304.046 | T-->5-Iodo-C | 110.900 |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5-Iodo-C | 85.894 |

Mass spectra of bioagent-identifying amplicons are analyzed independently using a maximum-likelihood processor, such as is widely used in radar signal processing. This processor, referred to as GenX, first makes maximum likelihood estimates of the input to the mass spectrometer for each primer by running matched filters for each base composition aggregate on the input data. This includes the GenX response to a calibrant for each primer.

The algorithm emphasizes performance predictions culminating in probability-of-detection versus probability-of-false-alarm plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bacterial bioagents and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent-identifying amplicons for each primer are calibrated and a final maximum likelihood amplitude estimate per organism is made based upon the multiple single primer estimates. Models of all system noise are factored into this two-stage maximum likelihood calculation. The processor reports the number of molecules of each base composition contained in the spectra. The quantity of amplification product corresponding to the appropriate primer set is reported as well as the quantities of primers remaining upon completion of the amplification reaction.

Base count blurring can be carried out as follows. “Electronic PCR” can be conducted on nucleotide sequences of the desired bioagents to obtain the different expected base counts that could be obtained for each primer pair. See for example, ncbi.nlm.nih.gov/sutils/e-pcr/; Schuler, Genome Res. 7:541-50, 1997. In one illustrative embodiment, one or more spreadsheets, such as Microsoft Excel workbooks contain a plurality of worksheets. First in this example, there is a worksheet with a name similar to the workbook name; this worksheet contains the raw electronic PCR data. Second, there is a worksheet named “filtered bioagents base count” that contains bioagent name and base count; there is a separate record for each strain after removing sequences that are not identified with a genus and species and removing all sequences for bioagents with less than 10 strains. Third, there is a worksheet that contains the frequency of substitutions, insertions, or deletions for this primer pair. This data is generated by first creating a pivot table from the data in the “filtered bioagents base count” worksheet and then executing an Excel VBA macro. The macro creates a table of differences in base counts for bioagents of the same species, but different strains. One of ordinary skill in the art may understand additional pathways for obtaining similar table differences without undo experimentation.

Application of an exemplary script, involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by taking the most abundant strain's base type composition and adding it to the reference set and then the next most abundant strain's base type composition is added until the threshold is met or exceeded. The current set of data was obtained using a threshold of 55%, which was obtained empirically.

For each base count not included in the reference base count set for that bioagent, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules that aim to minimize the number of changes and, in instances with the same number of changes, minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as one, two, or more substitutions, one, two, or more insertions, one, two, or more deletions, and combinations of substitutions and insertions or deletions. The different classes of nucleobase changes and their probabilities of occurrence have been delineated in U.S. Patent Application Publication No. 2004209260 which is incorporated herein by reference in entirety.

### Example 11

### Selection and Use of Primer Pairs for Identification of Species of Bacteria Involved in Sepsis

In this example, identification of bacteria known to cause sepsis was accomplished using a panel of primer pairs chosen specifically with the aim of identifying these bacteria (Table 8). In this current example, the more specific group of bacteria known to be involved in causing sepsis is to be surveyed. Therefore, in development of this current panel of primer pairs, certain established surveillance primer pairs of U.S. application Ser. No. 11/409,535 have been combined with an additional primer pair, primer pair number 2249. The primer members of primer pair 2249 hybridize to the tufB gene and produce a bioagent identifying amplicon for members of the family Staphylococcaceae which includes the genus Staphylococcus.

TABLE 8
Names of Primer Pairs in Panel for Characterization
of Septicemia Pathogens

| | | | Forward | | | Reverse |
|---|---|---|---|---|---|---|
| Primer | | Forward | Primer | | Reverse | Primer |
| Pair | Forward | Primer | (SEQ ID | Reverse | Primer | (SEQ ID |
| No. | Primer Name | Sequence | NO:) | Primer Name | Sequence | NO:) |
| 346 | 16S_EC_713_{—} | TAGAACACCG | 594 | 16S_EC_789_{—} | TCGTGGACT | 602 |
| | 732_TMOD_F | ATGGCGAAGGC | | 809_TMOD_R | ACCAGGGT | |
| | | | | | ATCTA | |
| 348 | 16S_EC_785_{—} | TTTCGATGCA | 595 | 16S_EC_880_{—} | TACGAGCTG | 603 |
| | 806_TMOD_F | ACGCGAAGA | | 897_TMOD_R | ACGACAGC | |
| | | ACCT | | | CATG | |
| 349 | 23S_EC_1826_{—} | TCTGACACCT | 596 | 23S_EC_1906_{—} | TGACCGTT | 604 |
| | 1843_TMOD_F | GCCCGGTGC | | 1924_TMOD_R | ATAGTTAC | |
| | | | | | GGCC | |
| 354 | RPOC_EC_{—} | TCTGGCAGGT | 597 | RPOC_EC_{—} | TCGCACCG | 605 |
| | 2218_2241_{—} | ATGCGTGGTC | | 2313_2337_{—} | TGGGTTGAG | |
| | TMOD_F | TGATG | | TMOD_R | ATGAAGTAC | |
| 358 | VALS_EC_{—} | TCGTGGCGGCG | 598 | VALS_EC_{—} | TCGGTACGA | 606 |
| | 1105_1124_{—} | TGGTTATCGA | | 1195_1218_{—} | ACTGGATGT | |
| | TMOD_F | | | TMOD_R | CGCCGTT | |
| 359 | RPOB_EC_{—} | TTATCGCTCAGG | 599 | RPOB_EC_{—} | TGCTGGATT | 607 |
| | 1845_1866_{—} | CGAACTCCAAC | | 1909_1929_{—} | CGCCTTTG | |
| | TMOD_F | | | TMOD_R | CTACG | |
| 449 | RPLB_EC_{—} | TCCACACGGTG | 600 | RPLB_EC_{—} | TGTGCTGGT | 608 |
| | 690_710_F | GTGGTGAAGG | | 737_758_R | TTACCCCA | |
| | | | | | TGGAG | |
| 2249 | TUFB_{—} | TGAACGTGGTC | 601 | TUFB_{—} | TGTCACCAG | 609 |
| | NC002758- | AAATCAAAGTT | | NC002758- | CTTCAGCGTA | |
| | 615038-616222_{—} | GGTGAAGA | | 615038-616222_{—} | GTCTAATAA | |
| | 696_725_F | | | 793_820_R | | |

To test for potential interference of human DNA with the present assay, varying amounts of bacterial DNA from E. coli 0157 and E. coli K-12 were spiked into samples of human DNA at various concentration levels. Amplification was carried out using primer pairs 346, 348, 349, 354, 358 and 359 and the amplified samples were subjected to gel electrophoresis. Smearing was absent on the gel, indicating that the primer pairs are specific for amplification of the bacterial DNA and that performance of the primer pairs is not appreciably affected in the presence of high levels of human DNA such as would be expected in blood samples. Measurement of the amplification products indicated that E. coli 0157 could be distinguished from E. coli K-12 by the base compositions of amplification products of primer pairs 358 and 359. This is a useful result because E. coli 0157 is a sepsis pathogen and because E. coli K-12 is a low-level contaminant of the commercially obtained Taq polymerase used for the amplification reactions. A test of 9 blinded mixture samples was conducted as an experiment designed to simulate a potential clinical situation where bacteria introduced via skin or oral flora contamination could confound the detection of sepsis pathogens. The samples contained mixtures of sepsis-relevant bacteria at different concentrations, whose identities were not known prior to measurements. Tables 9A and 9B show the results of the observed base compositions of the amplification products produced by the primer pairs of Table 8 which were used to identify the bacteria in each sample. Without prior knowledge of the bacteria included in the 9 samples provided, it was found that samples 1-5 contained Proteus mirabilis, Staphylococcus aureus, and Streptococcus pneumoniae at variable concentration levels as indicated in Tables 9A and 9B. Sample 6 contained only Staphylococcus aureus. Sample 7 contained only Streptococcus pneumoniae. Sample 8 contained only Proteus mirabilis. Sample 9 was blank.

Quantitation of the three species of bacteria was carried out using calibration polynucleotides as described herein. The levels of each bacterium quantitated for each sample was found to be consistent with the levels expected.

This example indicates that the panel of primer pairs indicated in Table 8 is useful for identification of bacteria that cause sepsis.

In another experiment, two blinded samples were provided. The first sample, labeled “Germ A” contained Enterococcus faecalis and the second sample, labeled “Germ B” contained other Klebsiella pneumoniae. For “Germ A” the panel of primer pairs of Table 8 produced four bioagent identifying amplicons from bacterial DNA and primer pair numbers 347, 348, 349 and 449 whose base compositions indicated the identity of “Germ A” as Enterococcus faecalis. For “Germ B” the panel of primer pairs of Table 8 produced six bioagent identifying amplicons from bacterial DNA and primer pair numbers 347, 348, 349, 358, 359 and 354 whose base compositions indicated the identity of “Germ B” as Klebsiella pneumoniae.

One with ordinary skill in the art will recognize that one or more of the primer pairs of Table 8 could be replaced with one or more different primer pairs should the analysis require modification such that it would benefit from additional bioagent identifying amplicons that provide bacterial identification resolution for different species of bacteria and strains thereof

TABLE 9A
Observed Base Compositions of Blinded Samples of Amplification Products Produced with Primer Pair Nos. 346, 348, 349 and 449

| | | Organism | | | | |
|---|---|---|---|---|---|---|
| | Organism | Concentration | Primer Pair | Primer Pair | Primer Pair | Primer Pair |
| Sample | Component | (genome copies) | Number 346 | Number 348 | Number 349 | Number 449 |
| 1 | Proteus mirabilis | 470 | A29G32C25T13 | — | — | — |
| 1 | Staphylococcus aureus | >1000 | — | A30G29C30T29 | A26G3C25T20 | — |
| 1 | Streptococcus pneumoniae | >1000 | — | A26G32C28T30 | A28G31C22T20 | A22G20C19T14 |
| 2 | Staphylococcus aureus | >1000 | A27G30C21T21 | A30G29C30T29 | A26G30C25T20 | — |
| 2 | Streptococcus pneumoniae | >1000 | — | — | — | A22G20C19T14 |
| 2 | Proteus mirabilis | 390 | — | — | — | — |
| 3 | Proteus mirabilis | >10000 | A29G32C25T13 | A29G30C28T29 | A25G31C27T20 | — |
| 3 | Streptococcus pneumoniae | 675 | — | — | — | A22G20C19T14 |
| 3 | Staphylococcus aureus | 110 | — | — | — | — |
| 4 | Proteus mirabilis | 2130 | A29G32C25T13 | A29G30C28T29 | A25G31C27T20 | — |
| 4 | Streptococcus pneumoniae | >3000 | — | A26G32C28T30 | A28G31C22T20 | A22G20C19T14 |
| 4 | Staphylococcus aureus | 335 | — | — | — | — |
| 5 | Proteus mirabilis | >10000 | A29G32C25T13 | A29G30C28T29 | A25G31C27T20 | — |
| 5 | Streptococcus pneumoniae | 77 | — | — | — | A22G20C19T14 |
| 5 | Staphylococcus aureus | >1000 | | | | |
| 6 | Staphylococcus aureus | 266 | A27G30C21T21 | A30G29C30T29 | A26G30C25T20 | — |
| 6 | Streptococcus pneumoniae | 0 | | — | — | — |
| 6 | Proteus mirabilis | 0 | — | — | — | — |
| 7 | Streptococcus pneumoniae | 125 | — | A26G32C28T30 | A28G31C22T20 | A22G20C19T14 |
| 7 | Staphylococcus aureus | 0 | — | — | — | — |
| 7 | Proteus mirabilis | 0 | — | — | — | — |
| 8 | Proteus mirabilis | 240 | A29G32C25T13 | A29G30C28T29 | A25G31C27T20 | — |
| 8 | Streptococcus pneumoniae | 0 | — | — | — | — |
| 8 | Staphylococcus aureus | 0 | — | — | — | — |
| 9 | Proteus mirabilis | 0 | — | — | — | — |
| 9 | Streptococcus pneumoniae | 0 | — | — | — | — |
| 9 | Staphylococcus aureus | 0 | — | — | — | — |

TABLE 9B
Observed Base Compositions of Blinded Samples of Amplification Products Produced
with Primer Pair Nos. 358, 359, 354 and 2249

| | | Organism | | | | |
|---|---|---|---|---|---|---|
| | Organism | Concentration | Primer Pair | Primer Pair | Primer Pair | Primer Pair |
| Sample | Component | (genome copies) | Number 358 | Number 359 | Number 354 | Number 2249 |
| 1 | Proteus mirabilis | 470 | — | — | A29G29C35T29 | — |
| 1 | Staphylococcus aureus | >1000 | — | — | A30G27C30T35 | A43G28C19T35 |
| 1 | Streptococcus pneumoniae | >1000 | — | — | — | — |
| 2 | Staphylococcus aureus | >1000 | — | — | A30G27C30T35 | A43G28C19T35 |
| 2 | Streptococcus pneumoniae | >1000 | — | — | — | — |
| 2 | Proteus mirabilis | 390 | — | — | A29G29C35T29 | — |
| 3 | Proteus mirabilis | >10000 | — | — | A29G29C35T29 | — |
| 3 | Streptococcus pneumoniae | 675 | — | — | — | — |
| 3 | Staphylococcus aureus | 110 | — | — | — | A43G28C19T35 |
| 4 | Proteus mirabilis | 2130 | — | — | A29G29C35T29 | — |
| 4 | Streptococcus pneumoniae | >3000 | — | — | — | — |
| 4 | Staphylococcus aureus | 335 | — | — | — | A43G28C19T35 |
| 5 | Proteus mirabilis | >10000 | — | — | A29G29C35T29 | — |
| 5 | Streptococcus pneumoniae | 77 | — | — | — | — |
| 5 | Staphylococcus aureus | >1000 | — | — | — | A43G28C19T35 |
| 6 | Staphylococcus aureus | 266 | — | — | — | A43G28C19T35 |
| 6 | Streptococcus pneumoniae | 0 | — | — | — | — |
| 6 | Proteus mirabilis | 0 | — | — | — | — |
| 7 | Streptococcus pneumoniae | 125 | — | — | — | — |
| 7 | Staphylococcus aureus | 0 | — | — | — | — |
| 7 | Proteus mirabilis | 0 | — | — | — | — |
| 8 | Proteus mirabilis | 240 | — | — | A29G29C35T29 | — |
| 8 | Streptococcus pneumoniae | 0 | — | — | — | — |
| 8 | Staphylococcus aureus | 0 | — | — | — | — |
| 9 | Proteus mirabilis | 0 | — | — | — | — |
| 9 | Streptococcus pneumoniae | 0 | — | — | — | — |
| 9 | Staphylococcus aureus | 0 | — | — | — | — |

### Example 12

### Design and Validation of Primer Pairs Designed for Production of Amplification Products from DNA of Sepsis-Causing Bacteria

The following primer pairs of Table 10 were designed to provide an improved collection of bioagent identifying amplicons for the purpose of identifying sepsis-causing bacteria.

TABLE 10
Primer Pairs for Producing Bioagent Identifying Amplicons
of Sepsis-Causing Bacteria

| Primer | | | Forward | | | Reverse |
|---|---|---|---|---|---|---|
| Pair | Forward | Forward | SEQ ID | Reverse | Reverse | SEQ ID |
| Number | Primer Name | Sequence | NO: | Primer Name | Sequence | NO: |
| 3346 | RPOB_{—} | TGAACCACT | 616 | RPOB_{—} | TCACCGAAACGC | 627 |
| | NC000913_{—} | TGGTTGACGA | | NC000913_{—} | TGACCACCGAA | |
| | 3704_3731_F | CAAGATGCA | | 3793_3815_R | | |
| 3347 | RPOB_{—} | TGAACCACTT | 616 | RPOB_{—} | TCCATCTCACCG | 632 |
| | NC000913_{—} | GGTTGACGA | | NC000913_{—} | AAACGCTGA | |
| | 3704_3731_F | CAAGATGCA | | 3796_3821_R | CCACC | |
| 3348 | RPOB_{—} | TGTTGATGA | 623 | RPOB_{—} | TCCATCTCACC | 632 |
| | NC000913_{—} | CAAGATGCA | | NC000913_{—} | GAAACGCTGA | |
| | 3714_3740_F | CGCGCGTTC | | 3796_3821_R | CCACC | |
| 3349 | RPOB_{—} | TGACAAGA | 619 | RPOB_{—} | CTCACCGAAACGCT | 636 |
| | NC000913_{—} | TGCACGCG | | NC000913_{—} | ACCACC | |
| | 3720_3740_F | CGTTC | | 3796_3817_R | | |
| 3350 | RPLB_EC_{—} | TCCACACGG | 614 | RPLB_{—} | TCCAAGCGCAG | 630 |
| | 690_710_F | TGGTGGT | | NC000913_{—} | GTTTACCCC | |
| | | GAAGG | | 739_762_R | ATGG | |
| 3351 | RPLB_EC_{—} | TCCACACGG | 614 | RPLB_{—} | TCCAAGCGCAG | 628 |
| | 690_710_F | TGGTGGT | | NC000913_{—} | GTTTACCCCA | |
| | | GAAGG | | 742_762_R | | |
| 3352 | RPLB_{—} | TGAACCCTA | 618 | RPLB_{—} | TCCAAGCGCAGG | 630 |
| | NC000913_{—} | ATGATCAC | | NC000913_{—} | TTTACCCCATGG | |
| | 674_698_F | CCACACGG | | 739_762_R | | |
| 3353 | RPLB_{—} | TGAACCCTAA | 617 | RPLB_{—} | TCCAAGCGCA | 629 |
| | NC000913_{—} | CGATCACC | | NC000913_{—} | GGTTTACCCCA | |
| | 674_698_2_F | CACACGG | | 742_762_R | | |
| 3354 | RPLB_EC_{—} | TCCACACGG | 614 | RPLB_{—} | TCCAAGCGCT | 631 |
| | 690_710_F | TGGTGGTG | | NC000913_{—} | GGTTTACCCCA | |
| | | AAGG | | 742_762_2_R | | |
| 3355 | RPLB_{—} | TCCAACTGTTC | 613 | RPLB_{—} | TCCAAGCGCAG | 630 |
| | NC000913_ [symbol] _{—} | GTGGTTCTGT | | NC000913_{—} | GTTTACCCC | |
| | 680_F | AATGAACCC | | 739_762_R | ATGG | |
| 3356 | RPOB_{—} | TCAGTTCGGT | 610 | RPOB_{—} | TACGTCGTCCG | 625 |
| | NC000913_{—} | GGCCAGCGC | | NC000913_{—} | ACTTGACCG | |
| | 3789_3812_F | TTCGG | | 3868_3894_R | TCAGCAT | |
| 3357 | RPOB_{—} | TCAGTTCGG | 610 | RPOB_{—} | TCCGACTTGAC | 633 |
| | NC000913_{—} | TGGCCAGC | | NC000913_{—} | CGTCAGCAT | |
| | 3789_3812_F | GCTTCGG | | 3862_3887_R | CTCCTG | |
| 3358 | RPOB_{—} | TCAGTTCGG | 611 | RPOB_{—} | TCGTCGGACTT | 635 |
| | NC000913_{—} | TGGTCAGCG | | NC000913_{—} | GATGGTCAGC | |
| | 3789_3812_2_F | CTTCGG | | 3862_3890_R | AGCTCCTG | |
| 3359 | RPOB_{—} | TCCACCGGTC | 615 | RPOB_{—} | CCGAAGCGCTG | 624 |
| | NC000913_{—} | CGTACTCC | | NC000913_{—} | GCCACCGA | |
| | 3739_3761_F | ATGAT | | 3794_3812_R | | |
| 3360 | GYRB_{—} | TCATACTCA | 612 | GYRB_{—} | TGCAGTCAAGC | 637 |
| | NC002737_{—} | TGAAGGTGG | | NC002737_{—} | CTTCACGAA | |
| | 852_879_F | AACGCATGAA | | 973_996_R | CATC | |
| 3361 | TUFB_{—} | TGATCACTG | 620 | TUFB_{—} | TGGATGTGTTC | 638 |
| | NC002758_{—} | GTGCTGCTC | | NC002758_{—} | ACGAGTTTGA | |
| | 275_298_F | AAATGG | | 337_362_R | GGCAT | |
| 3362 | VALS_{—} | TGGCGACCG | 621 | VALS_{—} | TACTGCTTCGG | 626 |
| | NC000913_{—} | TGGCGGCGT | | NC000913_{—} | GACGAACTG | |
| | 1098_1115_F | | | 1198_1226_R | GATGTCGCC | |
| 3363 | VALS_{—} | TGTGGCGGCG | 622 | VALS_{—} | TCGTACTGCTT | 634 |
| | NC000913_{—} | TGGTTATCG | | NC000913_{—} | CGGGACGA | |
| | 1105_1127_F | AACC | | 1207_1229_R | ACTG | |

[symbol]

indicates data missing or illegible when filed

Primer pair numbers 3346-3349, and 3356-3359 have forward and reverse primers that hybridize to the rpoB gene of sepsis-causing bacteria. The reference gene sequence used in design of these primer pairs is an extraction of nucleotide residues 4179268 to 4183296 from the genomic sequence of E. coli K12 (GenBank Accession No. NC 000913.2, gi number 49175990). All coordinates indicated in the primer names are with respect to this sequence extraction. For example, the forward primer of primer pair number 3346 is named RPOB_NC000913_{—}3704_{—}3731 F (SEQ ID NO: 616). This primer hybridizes to positions 3704 to 3731 of the extraction or positions 4182972 to 4182999 of the genomic sequence. Of this group of primer pairs, primer pair numbers 3346-3349 were designed to preferably hybridize to the rpoB gene of sepsis-causing gamma proteobacteria. Primer pairs 3356 and 3357 were designed to preferably hybridize to the rpoB gene of sepsis-causing beta proteobacteria, including members of the genus Neisseria, Primer pairs 3358 and 3359 were designed to preferably hybridize to the rpoB gene of members of the genera Corynebacterium and Mycobacterium. Primer pair numbers 3350-3355 have forward and reverse primers that hybridize to the rp1B gene of gram positive sepsis-causing bacteria. The forward primer of primer pair numbers 3350, 3351 and 3354 is RPLB_EC_{—}690_{—}710_F (SEQ ID NO: 614). This forward primer had been previously designed to hybridize to GenBank Accession No. NC_{—}000913.1, gi number 16127994. The reference gene sequence used in design of the remaining primers of primer pair numbers 3350-3355 is the reverse complement of an extraction of nucleotide residues 3448565 to 3449386 from the genomic sequence of E. coli K12 (GenBank Accession No. NC_{—}000913.2, gi number 49175990). All coordinates indicated in the primer names are with respect to the reverse complement of this sequence extraction. For example, the forward primer of primer pair number 3352 is named RPLB_NC000913_{—}674_{—}698_F (SEQ ID NO: 634). This primer hybridizes to positions 674-698 of the reverse complement of the extraction or positions 3449239 to 3449263 of the reverse complement of the genomic sequence. This primer pair design example demonstrates that it may be useful to prepare new combinations of primer pairs using previously existing forward or reverse primers.

Primer pair number 3360 has a forward primer and a reverse primer that both hybridize to the gyrB gene of sepsis-causing bacteria, preferably members of the genus Streptococcus. The reference gene sequence used in design of these primer pairs is an extraction of nucleotide residues 581680 to 583632 from the genomic sequence of Streptococcus pyogenes M1 GAS (GenBank Accession No. NC_{—}002737.1, gi number 15674250). All coordinates indicated in the primer names are with respect to this sequence extraction. For example, the forward primer of primer pair number 3360 is named GYRB_NC002737_{—}852_{—}879_F (SEQ ID NO: 612). This primer hybridizes to positions 852 to 879 of the extraction.

Primer pair number 3361 has a forward primer and a reverse primer that both hybridize to the tufB gene of sepsis-causing bacteria, preferably gram positive bacteria. The reference gene sequence used in design of these primer pairs is an extraction of nucleotide residues 615036 . . . 616220 from the genomic sequence of Staphylococcus aureus subsp. aureus Mu50 (GenBank Accession No. NC_{—}002758.2, gi number 57634611). All coordinates indicated in the primer names are with respect to this sequence extraction. For example, the forward primer of primer pair number 3361 is named TUFB_NC002758_{—}275_{—}298_F (SEQ ID NO: 612). This primer hybridizes to positions 275 to 298 of the extraction.

Primer pair numbers 3362 and 3363 have forward and reverse primers that hybridize to the valS gene of sepsis-causing bacteria, preferably including Klebsiella pneumoniae and strains thereof. The reference gene sequence used in design of these primer pairs is the reverse complement of an extraction of nucleotide residues 4479005 to 4481860 from the genomic sequence of E. coli K12 (GenBank Accession No. NC_{—}000913.2, gi number 49175990). All coordinates indicated in the primer names are with respect to the reverse complement of this sequence extraction. For example, the forward primer of primer pair number 3362 is named VALS_NC000913_{—}1098_{—}1115_F (SEQ ID NO: 621). This primer hybridizes to positions 1098 to 1115 of the reverse complement of the extraction.

In a validation experiment, samples containing known quantities of known sepsis-causing bacteria were prepared. Total DNA was extracted and purified in the samples and subjected to amplification by PCR according to Example 2 and using the primer pairs described in this example. The three sepsis-causing bacteria chosen for this experiment were Enterococcus faecalis, Klebsiella pneumoniae, and Staphylococcus aureus. Following amplification, samples of the amplified mixture were purified by the method described in Example 3 subjected to molecular mass and base composition analysis as described in Example 4.

Amplification products corresponding to bioagent identifying amplicons for Enterococcus faecalis were expected for primer pair numbers 3346-3355, 3360 and 3361. Amplification products were obtained and detected for all of these primer pairs.

Amplification products corresponding to bioagent identifying amplicons for Klebsiella pneumoniae were expected and detected for primer pair numbers 3346-3349, 3356, 3358, 3359, 3362 and 3363. Amplification products corresponding to bioagent identifying amplicons for Klebsiella pneumoniae were detected for primer pair numbers 3346-3349 and 3358. Amplification products corresponding to bioagent identifying amplicons for Staphylococcus aureus were expected and detected for primer pair numbers 3348, 3350-3355, 3360, and 3361. Amplification products corresponding to bioagent identifying amplicons for Klebsiella pneumoniae were detected for primer pair numbers 3350-3355 and 3361.

### Example 13

### Selection of Primer Pairs for Genotyping of Members of the Bacterial Genus Mycobacterium and for Identification of Drug-Resistant Strains of Mycobacterium tuberculosis

To combine the power of high-throughput mass spectrometric analysis of bioagent identifying amplicons with the sub-species characteristic resolving power provided by genotyping analysis and codon base composition analysis, a panel of twenty-four genotyping analysis primer pairs was selected. The primer pairs are designed to produce bioagent identifying amplicons within sixteen different housekeeping genes indicated by primer name codes in Table 11; rpoB, embB, fabG-inhA, katG, gyrA, rpsL, pncA, rv2109c, rv2348c, rv3815c, rv0041, rv00147, rv1814, rv0005gyrB, and rv0260c. The primer sequences are listed in Table 11.

In Mycobacterium tuberculosis, the acquisition of drug resistance is mostly associated with the emergence of discrete key mutations that can be unambiguously determined using the methods disclosed herein.

The evolution of the Mycobacterium tuberculosis genome is essentially clonal, thus allowing strain typing through the query of distinct genomic markers that are lineage-specific and only vertically inherited. Co-infections of mixed populations of genotypes of Mycobacterium tuberculosis can be revealed simultaneously in the mass spectra of amplification products produced using the primers of Table 11. The high G+C content and of the Mycobacterium tuberculosis genome itself greatly facilitates the development of short, efficient primers which are appropriate for multiplexing (inclusion of a plurality of primers in each amplification reaction mixture).

TABLE 11
Primer Pairs for Genotyping and Determination of Drug Resistance
of Strains of Mycobacterium tuberculosis

| | | | Forward | | | Reverse |
|---|---|---|---|---|---|---|
| Primer | | Forward | Primer | | Reverse | Primer |
| Pair | Forward | Primer | (SEQ ID | Reverse | Primer | (SEQ ID |
| No. | Primer Name | Sequence | NO:) | Primer Name | Sequence | NO:) |
| 3546 | RPOB_{—} | TGTGGCCGCG | 670 | RPOB_L27989-1- | TAGCCCGGC | 694 |
| | L27989-1-5084_{—} | ATCAAGGAG | | 5084_2458_2474_R | ACGCTCAC | |
| | 2333_2351_F | | | | | |
| 3547 | RPOB_{—} | TCAGCCAGC | 671 | RPOB_L27989-1- | TCCGACAG | 695 |
| | L27989-1-5084_{—} | TGAGCCAATT | | 5084_2388_2407_R | CGGGTTGTTCTG | |
| | 2362_2384_F | CATG | | | | |
| 3548 | RPOB_{—} | TCGCTGTCGGG | 672 | RPOB_L27989-1- | TCCGACAGT | 696 |
| | L27989-1-5084_{—} | GTTGACC | | 5084_2418_2434_R | CGGCGCTT | |
| | 2397_2414_F | | | | | |
| 3550 | EMBB_{—} | TGCTCTGGCAT | 673 | EMBB_AY727532-1- | TGAAGGGAT | 697 |
| | AY727532-1- | GTCATCGGC | | 344_209_228_R | CCTCCGGGCTG | |
| | 344_100_119_F | | | | | |
| 3551 | EMBB_{—} | TGACGGCTACA | 674 | EMBB_AY727532-1- | TGCGTGGTC | 698 |
| | AY727532-1- | TCCTGGGC | | 344_160_176_R | GGCGACTC | |
| | 344_134_152_F | | | | | |
| 3552 | FABG-INHA- | TGCTCGTGGAC | 675 | FABG-INHA- | TCAGTGGCTGT | 699 |
| | PROMOTER_{—} | ATACCGA | | PROMOTER_{—} | GGCAGTCAC | |
| | U66801-1- | TTTCG | | U66801-1- | GGCAGTCAC | |
| | 993_169_191_F | | | 993_224_243_R | | |
| 3553 | KATG_U06268-1- | TCGGTAAGGAC | 676 | KATG_U06268-1- | TGTCCATACG | 700 |
| | 2324_991_1010_F | GCGATCACC | | 2324_1014_1034_R | ACCTCGATGCC | |
| 3554 | KATG_U06268-1- | TGCCAGCCTTA | 677 | KATG_U06268-1- | TGTGAGACAGTC | 701 |
| | 2324_1433_1454_F | AGAGCCAGATC | | 2324_1458_1480_R | AATCCCGATGC | |
| 3555 | GYRA_AF400983-1- | TCACCCGCAC | 678 | GYRA_AF400983-1- | TGGGCCA | 702 |
| | 385_69_84_F | GGCGAC | | 385_103_119_R | TGCGCACCAG | |
| 3556 | GYRA_AF400983-1- | TCGACGCGTCG | 679 | GYRA_AF400983-1- | TGGGCCATG | 702 |
| | 385_80_99_F | ATCTACGAC | | 385_103_119_R | CGCACCAG | |
| 3557 | RPSL_AY156733-1- | TGGCTCTGAAG | 680 | RPSL_AY156733-1- | TGCCGTGACCT | 703 |
| | 375_65_82_F | GGCAGCC | | 375_177_195_R | CGACCTGA | |
| 3558 | PNCA_AL123456.2_{—} | TCTGTGGCTGC | 681 | PNCA_AL123456.2_{—} | TCGGCGCCA | 704 |
| | gi41353971-1- | CGCGTC | | gi41353971-1- | CCGGTTAC | |
| | 4411532_2289165_{—} | | | 4411532_2289303_{—} | | |
| | 2289181_F (RC) | | | 2289287_R (RC) | | |
| 3559 | PNCA_AL123456.2_{—} | TCATCACGTCG | 682 | PNCA_AL123456.2_{—} | TACGTGTCCAG | 705 |
| | gi41353971-1- | TGGCAACCA | | gi41353971-1- | ACTGGGATGGA | |
| | 4411532_2288970_{—} | | | 4411532_2289119_{—} | | |
| | 2288989_F (RC) | | | 2289098_R (RC) | | |
| 3560 | PNCA_AL123456.2_{—} | TGTGCCTACAC | 683 | PNCA_AL123456.2_{—} | TCGTCTGGCGC | 706 |
| | gi41353971-1- | CGGAGCG | | gi41353971-1- | ACACAATGAT | |
| | 4411532_2288815_{—} | | | 4411532_2288953_{—} | | |
| | 2288832_F (RC) | | | 2288933_R (RC) | | |
| 3561 | PNCA_AL123456.2_{—} | TCCGATCATTG | 684 | PNCA_AL123456.2_{—} | TGGTGCGCATC | 707 |
| | gi41353971-1- | TGTGCGCCA | | gi41353971-1- | TCCTCCAG | |
| | 4411532_2288710_{—} | | | 4411532_2288839_{—} | | |
| | 2288729_F (RC) | | | 2288821_R (RC) | | |
| 3581 | RV2109C_{—} | TCGACCCGTC | 685 | RV2109C_AL123456.2_{—} | TGCCGAGGT | 708 |
| | AL123456.2_{—} | GTAGGTAATA | | gi41353971-1- | GGCGCATT | |
| | gi41353971-1- | CGATAC | | 4411532_2369342_{—} | | |
| | 4411532_2369291_{—} | | | 2369358_R | | |
| | 2369316_F | | | | | |
| 3582 | RV2348C_{—} | TGCCTGTTTGA | 686 | RV2348C_AL123456.2_{—} | TCGGGCTCAACG | 709 |
| | AL123456.2_{—} | AACTGCCCA | | gi41353971-1- | ACACTTCCT | |
| | gi41353971-1- | CATAC | | 4411532₋2627954_{—} | | |
| | 4411532_2627916_{—} | | | 2627974_R | | |
| | 2627940_F | | | | | |
| 3583 | RV3815C_{—} | TGCCTTGGTCG | 687 | RV3815C_AL123456.2_{—} | TCCACCGGAA | 710 |
| | NC000962-1- | GGCACATTC | | gi41353971-1- | CCCGGATCA | |
| | 4411532_4280680_{—} | | | 4411532₋4280716_{—} | | |
| | 4280699_F | | | 4280734_R | | |
| 3584 | RV0041_AL123456.2_{—} | TCTGCCCGCCG | 688 | RV0041_AL123456.2_{—} | TGGTCCGGGT | 711 |
| | gi41353971-1- | AGCAATAC | | gi41353971-1- | ACGCGGA | |
| | 4411532_43921_{—} | | | 4411532_43960_{—} | | |
| | 43939_F | | | 43976_R | | |
| 3586 | RV0147_AL123456.2_{—} | TCCGTAAGTC | 689 | RV0147_AL123456.2_{—} | TGGCGGGTAGA | 712 |
| | gi41353971-1- | GGTGTTGA | | gi41353971-1- | TAAAGCTGGACA | |
| | 4411532_174655_{—} | CCAAAC | | 411532_174694_{—} | | |
| | 174678_F | | | 174716_R | | |
| 3587 | RV1814_AL123456.2_{—} | TCGGGTCCACC | 690 | RV1814_AL123456.2_{—} | TGGATGCCGCC | 713 |
| | gi41353971-1- | ACGGAATG | | gi41353971-1- | ATAGTTCTTGTC | |
| | 4411532_2057117_{—} | | | 4411532_2057151_{—} | | |
| | 2057135_F | | | 2057173_R | | |
| 3599 | RV0083_AL123456.2_{—} | TGCCGACGCGA | 691 | RV0083_AL123456.2_{—} | TAACAGCTCGG | 714 |
| | gi41353971-1- | TCGAACAG | | gi41353971-1- | CCATGGCG | |
| | 4411532_92169_{—} | | | 4411532_{—} | | |
| | 92187_F | | | 92220_92238_R | | |
| 3600 | RV0005GYRB_{—} | TGACCAA | 692 | RV0005GYRB_{—} | TGAGGACACAG | 715 |
| | AL123456.2_{—} | GACC | | AL123456.2_{—} | CC | |
| | gi41353971-1- | AAGTTGGGCA | | gi41353971-1- | TTGTTCACA | |
| | 4411532_6348_{—} | | | 4411532_{—} | | |
| | 6368_F | | | 6457_6478_R | | |
| 3601 | RV0260C_AL123456.2_{—} | TGCCCAGAGC | 693 | RV0260C_AL123456.2_{—} | TACACCCACGCC | 716 |
| | gi41353971-1- | CGTTCGT | | gi41353971-1- | GTGGA | |
| | 4411532_311588_{—} | | | 4411532_311623_{—} | | |
| | 311604_F | | | 311639_2_R | | |

The panel of 24 primer pairs is designed to be multiplexed into 8 amplification reactions. Thirteen primer pairs were designed with the objective of identifying mutations associated with resistance to drugs including rifampin (primer pair numbers 3546, 3547 and 3548), ethambutol (primer pair numbers 3550 and 3551), isoniazid (primer pair numbers 3353 and 3354), fluoroquinolone (primer pair number 3556), streptomycin (primer pair number 3557) and pyrazinamide (primer pair numbers 3558, 3558, 3560 and 3561). Four of these thirteen primer pairs were specifically designed to provide bioagent identifying amplicons for base composition analysis of single codons (primer pair numbers 3547 (rpoB codon D526), 3548 (rpoB codon H516), 3551 (embB codon M306), and 3553 (katG codon S315)). In any of these bioagent identifying amplicons used for base composition analysis, detection of a mutation identifies a drug-resistant strain of Mycobacterium tuberculosis. The remaining nine primer pairs define larger bioagent identifying amplicons that contain secondary drug resistance-conferring sites which are more rare than the four codons discussed above, but certain of these nine primer pairs define bioagent identifying amplicons that also contain some of these four codons (for example, primer pair 3546 contains two rpoB codons; D526 and H516).

Shown in Table 12 are classifications of members of the bacterial genus Mycobacterium according to principal genetic group (PGG, determined using primer pair numbers X and X), genotype of Mycobacterium tuberculosis, or species of selected other members of the genus Mycobacterium (determined using primer pair numbers X, Y, Z), and drug resistance to rifampin, ethambutol, isoniazid, fluoroquinolone, streptomycin, and pyrazinamide. The primer pairs used to define the bioagent identifying amplicons for each PPG group, genotype or drug resistant strain are shown in the column headings. In the drug resistance columns, codon mutations are indicated by the amino acid single letter code and codon position convention which is well known to those with ordinary skill in the art. For example, when nucleic acid of Mycobacterium tuberculosis strain 13599 is amplified using primer pair number 3555, and the molecular mass or base composition is determined, mutation of codon 90 from alanine (A) to valine (V) is indicated and the conclusion is drawn that strain 13599 is resistant to the drug fluoroquinolone.

Primer pair number 3600 is a speciation primer pair which is useful for distinguishing members of Mycobacterium tuberculosis PPG1 (including genotypes I, II and HA) from other species of the genus Mycobacterium (such as for example, Mycobacterium africanum, Mycobacterium bovis, Mycobacterium microti, and Mycobacterium canettii).

TABLE 12
Classification and Drug Resistance Profiles of Strains of Members of the Genus Mycobacterium and Genotypes
of Mycobacterium tuberculosis

| | Principal | Genotype | | | | | | | |
|---|---|---|---|---|---|---|---|---|---|
| | Genetic | Primer Pair | Drug | | | | | | Drug |
| | Group | Numbers: | Resistance to | Drug | Drug | | Drug | Drug | Resistance to |
| | (PGG) | 3581, 3582, | Rifampin | Resistance to | Resistance to | | Resistance to | Resistance to | Pyrazinamide |
| | Primer | 3583, 3584, | Primer Pair | Ethambutol | Isoniazid | | Fluoroquinolone | Streptomycin | Primer Pair |
| | Pair | 3586, 3587, | Numbers: | Primer Pair | Primer Pair | | Primer Pair | Primer Pair | Numbers: |
| | Numbers: | 3599, 3600, | 3546, | Numbers: | Numbers: | | Number: | Number: | 3558, 3559, |
| Strain | 3554, 3556 | 3601 | 3547, 3548 | 3550, 3551 | 3553 | 3552 | 3555 | 3557 | 3560, 3561 |
| 19422 | PGG-1 | M africanum | wild type | wt | wt | wt | wt | wt | wt |
| | | or | | | | | | | |
| | | M. microti | | | | | | | |
| 10130 | PGG-1 | M. bovis | wt | wt | wt | wt | wt | wt | [part2] C > G |
| 35737 (BCG) | PGG-1 | M. bovis | wt | wt | wt | wt | wt | wt | wt |
| M. Canettii | PGG-1 | M. canettii | wt | wt | wt | wt | wt | wt | [part2] C > G |
| 14157, 15042 | PGG-1 | I | wt | wt | wt | wt | wt | wt | wt |
| 16116 | PGG-1 | IIA | wt | wt | wt | wt | wt | wt | wt |
| 15021 | PGG-1 | IIA | wt | wt | wt | wt | wt | wt | [part2] C > T |
| 5116 | PGG-1 | IIA | wt | wt | S315T | wt | wt | wt | wt |
| 12360, 13876, | PGG-1 | II | wt | wt | wt | wt | wt | wt | wt |
| 14149 | | | | | | | | | |
| 13599 | PGG-1 | II | wt | wt | wt | C-15T | A90V | wt | [part2] A > G |
| 13598 | PGG-1 | II | H528Y | M306V | S315 (N/T) | wt | wt | K43R | wt |
| 10545 | PGG-1 | II | wt | M306I | S315T | wt | wt | wt | wt |
| 13632 | PGG-1 | II | transition | M306I | S315T | wt | wt | wt | [part2] C > T, |
| | | | | | | | | | [part3] G > C |
| 14207 | PGG-1 | III | wt | wt | wt | wt | wt | wt | wt |
| 13866, 13874, | PGG-2 | III or IV | wt | wt | wt | wt | wt | wt | wt |
| 14038 | | | | | | | | | |
| 12578, 12590 | PGG-2 | III or IV | wt | wt | S315T | wt | wt | wt | [part3] G > C |
| 14404 | PGG-2 | IV | wt | wt | wt | wt | wt | wt | wt |
| 14831 | PGG-2 | IV | wt | wt | S315T | T-8C | wt | wt | wt |
| 5170, 13672, | PGG-2 | V | wt | wt | wt | wt | wt | wt | wt |
| 13699, 14424 | | | | | | | | | |
| 13679, 14399 | PGG-2 | VI | wt | wt | wt | wt | wt | wt | wt |
| 13592 | PGG-2 | VI | wt | wt | S315T | wt | wt | wt | wt |
| 13594, 13658, | PGG-3 | VII | wt | wt | wt | wt | T95S | wt | wt |
| 13869 | | | | | | | | | |
| 13821 | PGG-3 | VIII | wt | wt | wt | wt | T95S | wt | wt |
| 35837 (H37Rv7) | PGG-3 | VIII | wt | M306V | wt | wt | T95S | wt | wt |

### Example 14

### Validation of the Panel of 24 Primer Pairs

Each primer pair was individually validated using the reference Mycobacterium tuberculosis strain H37Rv. Dilution To Extinction (DTE) experiments yielded the expected base composition down to 16 genomic copies per well. A multiplexing scheme was then determined in order to spread into different wells the primer pairs targeting the same gene, to spread within a single well the expected amplicon masses, and to avoid cross-formation of primer duplexes. The multiplexing scheme is shown in Table 13 where multiplexed amplification reactions are indicated in headings numbered A through H and the primer pairs utilized for each reaction are shown below.

TABLE 13
Multiplexing Scheme for Panel of 24 Primer Pairs

| Reaction A | Reaction B | Reaction C | Reaction D | Reaction E | Reaction F | Reaction G | Reaction H |
|---|---|---|---|---|---|---|---|
| 3547 | 3548 | 3601 | 3551 | 3553 | 3554 | 3555 | 3556 |
| 3581 | 3584 | 3599 | 3582 | 3583 | 3587 | 3552 | 3586 |
| 3550 | 3600 | 3559 | 3560 | 3546 | 3558 | 3561 | 3557 |

An example of an experimentally determined table of base compositions is shown in Table 14. Base compositions of amplification products obtained from nucleic acid isolated from Mycobacterium tuberculosis strain 5170 using the primer pair multiplex reactions indicated in Table 13 are shown. Molecular masses of the amplification products were measured by electrospray time of flight mass spectrometry in order to calculate the base compositions. It should be noted that the lengths of the amplification products within each reaction mixture vary greatly in length in order to avoid overlap of molecular masses during the measurements. For example, reaction A has three amplification products which have lengths of 46 (A13 T11 C15 G07), 68 (A14 T18 C21 G15) and 129 (A21 T37 C44 G27).

TABLE 14
Base Compositions Obtained in the Multiplex Amplification Reactions
of Nucleic Acid of Mycobacterium tuberculosis Strain 5170

| | | Base Composition |
|---|---|---|
| Reaction | Primer Pair No. | (A G C T) |
| A | 3547 | 13 11 15 07 |
| A | 3581 | 14 18 21 15 |
| A | 3550 | 21 37 44 27 |
| B | 3548 | 06 13 12 07 |
| B | 3584 | 13 13 24 06 |
| B | 3600 | 37 34 35 25 |
| C | 3601 | 07 20 15 10 |
| C | 3599 | 10 26 22 12 |
| C | 3559 | 26 34 53 28 |
| D | 3551 | 08 13 16 06 |
| D | 3582 | 13 15 17 14 |
| D | 3560 | 28 48 37 26 |
| E | 3553 | 11 15 11 07 |
| E | 3583 | 06 19 16 14 |
| E | 3546 | — |
| F | 3554 | 11 13 14 10 |
| F | 3587 | 15 16 16 10 |
| F | 3558 | — |
| G | 3555 | 09 14 21 07 |
| G | 3552 | 13 26 22 14 |
| G | 3561 | 22 48 39 21 |
| H | 3556 | 07 11 15 07 |
| H | 3586 | 15 11 23 13 |
| H | 3557 | 26 44 39 22 |

Dilution to extinction experiments were then carried out with the chosen triplets of primer pairs in multiplex conditions. Base compositions expected on the basis of the known sequence of the reference strain were observed down to 32 genomic copies per well on average. The assay was finally tested using a collection of 36 diverse strains from the Public Health Research Institute. As expected, the base compositions results were in accordance with the genotyping and drug-resistance profiles already determined for these reference strains.

### Example 15

### Primer Pairs that Define Bioagent Identifying Amplicons for Hepatitis C Viruses

For design of primers that define hepatitis c virus strain identifying amplicons, a series of hepatitis C virus genome sequences were obtained, aligned and scanned for regions where pairs of PCR primers would amplify products of about 27 to about 200 nucleotides in length and distinguish strains and quasispecies from each other by their molecular masses or base compositions.

Table 15 represents a collection of primers (sorted by primer pair number) designed to identify hepatitis C viruses using the methods described herein. The primer pair number is an in-house database index number. The forward or reverse primer name shown in Table 15 indicates the gene region of the viral genome to which the primer hybridizes relative to a reference sequence. In Table 15, for example, the forward primer name HCVUTR5_NC001433-1-9616_{—}9250_{—}9273_F indicates that the forward primer CF) hybridizes to residues 9250-9275 of the UTR (untranslated region) of a hepatitis C virus reference sequence represented by an extraction of nucleotides 1 to 9616 of GenBank Accession No. NC_{—}001433.1. One with ordinary skill will know how to obtain individual gene sequences or portions thereof from genomic sequences present in GenBank.

TABLE 15
Primer Pairs for Identification of Strains of Hepatitis C Viruses

| Primer | | | Forward | | | Reverse |
|---|---|---|---|---|---|---|
| Pair | Forward | Forward | SEQ ID | Reverse | Reverse | SEQ ID |
| No. | Primer Name | Sequence | NO: | Primer Name | Sequence | NO: |
| 3682 | HCVUTR5_{—} | TCAGCGGA | 655 | HCVUTR5_{—} | TACTCCTCC | 662 |
| | NC001433-1-9616_{—} | GGTGACAT | | NC001433-1-9616_{—} | TTTCGGTA | |
| | 9250_9273_F | GTATCACA | | 9313_9337_R | GCGGTAGA | |
| 3683 | HCVUTR5_{—} | TCGACCAAC | 656 | HCVUTR5_{—} | GACATGTAT | 663 |
| | NC001433-1-9616_{—} | CTTAAACG | | NC001433-1-9616_{—} | CACAACCT | |
| | 9177_9200_F | CACTCCA | | 9261_9285_R | GTCGCACA | |
| 3684 | HCVUTR5_{—} | TTAGCACC | 657 | HCVUTR5_{—} | CATGCTAAT | 664 |
| | NC001433-1-9616_{—} | TCGACGG | | NC001433-1-9616_{—} | GTCGTTCC | |
| | 3644_3662_F | CTGG | | 3735_3756_R | GGCGA | |
| 3685 | HCVUTR5_{—} | TGCTCGGA | 658 | HCVUTR5_{—} | CATGCTGAT | 665 |
| | NC001433-1-9616_{—} | CCTTTACT | | NC001433-1-9616_{—} | GTCATTCCG | |
| | 3708_3731_F | TGGTCACG | | 3735_3757_R | GTGCA | |
| 3686 | HCVUTR5_{—} | TGCTCGGA | 658 | HCVUTR5_{—} | TCGGGTGGTC | 666 |
| | NC001433-1-9616_{—} | CCTTTAC | | NC001433-1-9616_{—} | CACTGCTCA | |
| | 3708_3731_F | TTGGTCACG | | 3822_3840_R | | |
| 3687 | HCVUTR5_{—} | TGCCCGT | 659 | HCVUTR5_{—} | GCTGTGTACAC | 667 |
| | NC001433-1-9616_{—} | CTCCTAC | | NC001433-1-9616_{—} | CCGGCGA | |
| | 3796_3817_F | TTGAAGGG | | 3876_3893_R | | |
| 3688 | HCVUTR5_{—} | TTTGCGG | 660 | HCVUTR5_{—} | GCTGTGTACAC | 667 |
| | NC001433-1-9616_{—} | GCACCTT | | NC001433-1-9616_{—} | CCGGCGA | |
| | 3855_3872_F | CCGG | | 3876_3893_R | | |
| 3689 | HCVUTR5_{—} | TTTGCGGG | 660 | HCVUTR5_{—} | ATGCGGTATCC | 668 |
| | NC001433-1-9616_{—} | CACCTT | | NC001433-1-9616_{—} | GGTCCTCACA | |
| | 3855_3872_F | CCGG | | 3942_3962_2_R | | |
| 3691 | HCVUTR5_{—} | TGGCTCGG | 661 | HCVUTR5_{—} | TGCCCAACGGA | 669 |
| | NC001433-1- | TTGTACAG | | NC001433-1- | CTACTTCCTGA | |
| | 9616_1974_1996_2_F | GGATGAA | | 9616_2070_2091 | | |

### Example 16

### Primer Pairs that Define Bioagent Identifying Amplicons for Identification of Strains of Influenza Viruses

For design of primers that define bioagent identifying amplicons for identification of strains of influenza viruses, a series of influenza virus genome sequences were obtained, aligned and scanned for regions where pairs of PCR primers would amplify products of about 27 to about 200 nucleotides in length and distinguish influenza virus strains of from each other by their molecular masses or base compositions.

Table 16 represents a collection of primers (sorted by primer pair number) designed to identify hepatitis C viruses using the methods described herein. The primer pair number is an in-house database index number. The forward or reverse primer name shown in Table 16 indicates the gene region of the influenza virus genome to which the primer hybridizes relative to a reference sequence. In Table 16, for example, the forward primer name FLUBPB2_NC002205_{—}603_{—}629_F indicates that the forward primer (F) hybridizes to residues 603-629 of an influenza reference sequence represented by an extraction of nucleotides from GenBank Accession No. NC_{—}002205. One with ordinary skill will know how to obtain individual gene sequences or portions thereof from genomic sequences present in GenBank.

TABLE 16
Primer Pairs for Identification of Strains of Influenza Viruses

| Primer | | | Forward | | | Reverse |
|---|---|---|---|---|---|---|
| Pair | Forward | Forward | SEQ ID | Reverse | Reverse | SEQ ID |
| Number | Primer Name | Sequence | NO: | Primer Name | Sequence | NO: |
| 1261 | FLUBPB2_{—} | TCCCATTGTAC | 639 | FLUBPB2_{—} | TATGAACTCA | 647 |
| | NC002205_603_{—} | TGGCATACA | | NC002205_667_{—} | GCTGATGTTG | |
| | 629_F | TGCTTGA | | 693_R | CTCCTGC | |
| 1266 | FLUANUC_{—} | TACATCCAGAT | 640 | FLUANUC_{—} | TCGTCAAATG | 648 |
| | J02147_118_{—} | GTGCACTGAAC | | J02147_188_{—} | CAGAGAGCAC | |
| | 148_F | TCAAACTCA | | 218_R | CATTCTCTCTA | |
| 1275 | FLUBNUC_{—} | TCCAATCATC | 641 | FLUBNUC_{—} | TCCGATATCAG | 649 |
| | NC002208_{—} | AGACCAGCAA | | NC002208_{—} | CTTCACTGC | |
| | 90_116_F | CCCTTGC | | 164_189_R | TTGTGG | |
| 1279 | FLUAM1_{—} | TCTTGCCAGTT | 642 | FLUAM1_{—} | TGGGAGTCAG | 650 |
| | NC004524_369_{—} | GTATGGGCCT | | NC004524_451_{—} | CAATCTGC | |
| | 396_F | CATATAC | | 473_R | TCACA | |
| 1287 | FLUAPA_{—} | TGGGATTCCTTT | 643 | FLUAPA_{—} | TGGAGAAGTT | 651 |
| | NC004520_{—} | CGTCAGTCCGA | | NC004520_{—} | CGGTGGGAG | |
| | 562_584_F | | | 647_673_R | ACTTTGGT | |
| 2775 | FLUANS1_{—} | TCCAGGACAT | 644 | FLUANS1_{—} | TGCTTCCCCA | 652 |
| | NC004525_1_{—} | ACTGATGAGGAT | | NC004525_29_{—} | AGCGAATCT | |
| | 19_F | GTCAAAAATGCA | | 52_R | CTGTA | |
| 2777 | FLUANS2_{—} | TGTCAAAAATG | 645 | FLUANS2_{—} | TCATTACTGCT | 653 |
| | NC004525_47_{—} | CAATTGGGGT | | NC004525_121_{—} | TCTCCAAGCGA | |
| | 74_F | CCTCATC | | 151_R | ATCTCTGTA | |
| 2798 | FLUPB1_{—} | TGTCCTGGAAT | 646 | FLU_ALL_{—} | TCATCAGAGG | 654 |
| | J02151_1210_{—} | GATGATGGGCA | | PB1_J02151_{—} | ATTGGAGTCCA | |
| | 1235_F | TGTT | | 1313_1337_R | TCCC | |
| 1261 | FLUBPB2_{—} | TCCCATTGTACT | 639 | FLUBPB2_{—} | TATGAACTCAG | 647 |
| | NC002205_603_{—} | GGCATACATG | | NC002205_667_{—} | CTGATGTTGCT | |
| | 629_F | CTTGA | | 693_R | CCTGC | |

### Example 17

### Primer Pairs that Define Bioagent Identifying Amplicons for Identification of Strains of Staphylococcus aureus

For design of primers that define bioagent identifying amplicons for identification of strains of Staphylococcus aureus, a series of Staphylococcus aureus virus genome sequences were obtained, aligned and scanned for regions where pairs of PCR primers would amplify products of about 27 to about 200 nucleotides in length and distinguish Staphylococcus aureus strains of from each other by their molecular masses or base compositions.

Table 17 represents a collection of primers (sorted by primer pair number) designed to identify Staphylococcus aureus strains using the methods described herein. The primer pair number is an in-house database index number. The forward or reverse primer name shown in Table 17 indicates the gene region of the influenza virus genome to which the primer hybridizes relative to a reference sequence. In Table 17, for example, the forward primer name MECA_Y14051_{—}4507_{—}4530_F indicates that the forward primer (F) hybridizes to residues 4507-4530 of the mecA gene of Staphylococcus aureus sequence represented by GenBank Accession No. Y14051. One with ordinary skill will know how to obtain individual gene sequences or portions thereof from genomic sequences present in GenBank.

TABLE 17
Primer Pairs for Identification of Strains of Staphylococcus aureus

| Primer | | | Forward | | | Reverse |
|---|---|---|---|---|---|---|
| Pair | Forward | Forward | SEQ ID | Reverse | Reverse | SEQ ID |
| Number | Primer Name | Sequence | NO: | Primer Name | Sequence | NO: |
| 879 | MECA_Y14051_{—} | TCAGGTACTG | 717 | MECA_Y14051_{—} | TGGATAGACGT | 727 |
| | 4507_4530_F | CTATCCACCC | | 4555_4581_R | CATATGAAG | |
| | | TCAA | | | GTGTGCT | |
| 2056 | MECI-R_{—} | TTTACACATAT | 718 | MECI-R_{—} | TGTGATATGGAGGT | 728 |
| | NC003923-41798- | CGTGAGCAAT | | NC003923-41798- | TAGAAGGTGTTA | |
| | 41609_33_60_F | GAACTGA | | 41609_86_113_R | | |
| 2081 | ERMA_{—} | AGCTATCTTATCGT | 719 | ERMA_{—} | TGAGCATTTTTA | 729 |
| | NC002952-55890- | [symbol] AGAAGGGATTT | | NC002952-55890- | TATCCATCT | |
| | 56621_366_395_F | G [symbol] | | 56621_438_465_R | CCACCAT | |
| 2086 | ERMC_{—} | TCTGAACATGA | 720 | ERMC_{—} | TCCGTAGTTTTG | 730 |
| | NC005908-2004- | TAATATCTTTGA | | NC005908-2004- | CATAATTTATG | |
| | 2738_85_116_F | AATCGGCTC | | 2738 173 206R | GTCTATTTCAA | |
| 2095 | PVLUK_{—} | TGAGCTGCATC | 721 | PVLUK_{—} | TGGAAAACTCA | 731 |
| | NC003923-1529595- | AACTGTATT | | NC003923- | TGAAATTAAA | |
| | 1531285_688_713_F | GGATAG | | 1529595-1531285_{—} | GTGAAAGGA | |
| | | | | 775_804_R | | |
| 2256 | NUC_NC002758- | TACAAAGGTC | 722 | NUC_NC002758- | TAAATGCACTT | 732 |
| | 894288- | AACCAATGAC | | 894288-894974_{—} | GCTTCAGGG | |
| | 894974_316_345_F | ATTCAGACTA | | 396_421_R | CCATAT | |
| 2313 | MUPR_X75439_{—} | TAATTGGGCTC | 723 | MUPR_X75439_{—} | TAATCTGGCTGCGG | 733 |
| | 2486_2516_F | TTTCTCGCTTA | | 2548_2574_R | AGTGAAATCGT | |
| | | AACACCTTA | | | | |
| 3005 | TUFB_NC002758- | TGCCGTGTTG | 724 | TUFB_NC002758- | TGCTTCAGCGT | 734 |
| | 615038-616222_{—} | AACGTGGTC | | 615038-616222_{—} | AGTCTAATAAT | |
| | 688_710_F | AAAT | | 783_813_R | TTACGGAAC | |
| 3016 | MUPR_X75439_{—} | TAGATAATTG | 725 | MUPR_X75439_{—} | AATCTGGCTGCGGA | 735 |
| | 2482_2510_F | GGCTCTTTCTC | | 2551_2573_R | GTGAAAT | |
| | | GCTTAAAC | | | | |
| 3106 | TSST1_NC002758.2_{—} | TCGTCATCAG | 726 | TSST1_{—} | TCACTTTGATAT | 736 |
| | 519_546_F | CTAACTCAAA | | NC002758.2_{—} | GTGGATCCGT | |
| | | TACATGGA | | 593_620_R | CATTCA | |
| 2738 | GYRA_NC002953- | TAAGGTATGAC | 737 | GYRA_{—} | TCTTGAGCCATA | 740 |
| | 7005-9668_{—} | ACCGGATAAA | | NC002953-7005- | CGTACCATTGC | |
| | 166_195_F | TCATATAAA | | 9668_265_287_R | | |
| 2739 | GYRA_NC002953- | TAATGGGTAAA | 738 | GYRA_{—} | TATCCATTGAAC | 741 |
| | 7005-9668_221_{—} | TATCACCCTC | | NC002953-7005- | CAAAGTTACCT | |
| | 249_F | ATGGTGAC | | 9668_316_343_R | TGGCC | |
| 2740 | GYRA_NC002953- | TAATGGGTAAA | 738 | GYRA_{—} | TAGCCATACGTA | 742 |
| | 7005-9668_{—} | TATCACCCTC | | NC002953-7005- | CCATTGCTTCA | |
| | 221_249_F | ATGGTGAC | | 9668_253_283_R | TAAATAGA | |
| 2741 | GYRA_NC002953- | TCACCCTCATG | 739 | GYRA_{—} | TCTTGAGCCATA | 740 |
| | 7005-9668_{—} | GTGACTCATC | | NC002953-7005- | CGTACCATTGC | |
| | 234_261_F | TATTTAT | | 9668_265_287_R | | |

[symbol]

indicates data missing or illegible when filed

### Example 18

### Comparison of Targeted Whole Genome Amplification Method with an Unbiased Whole Genome Amplification Method

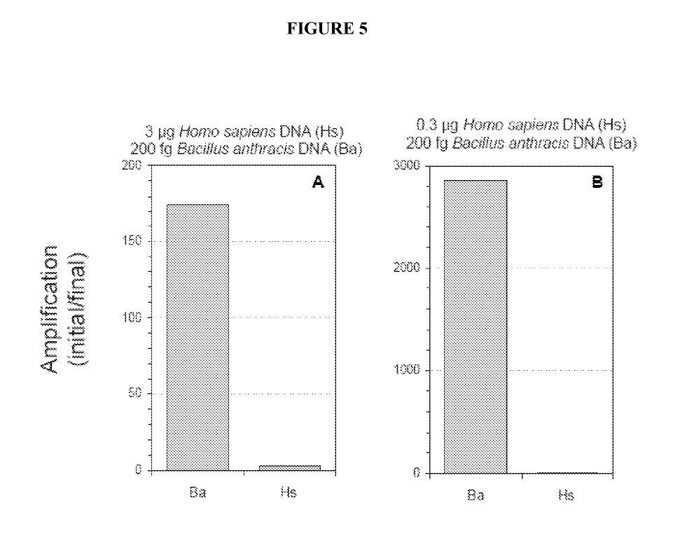

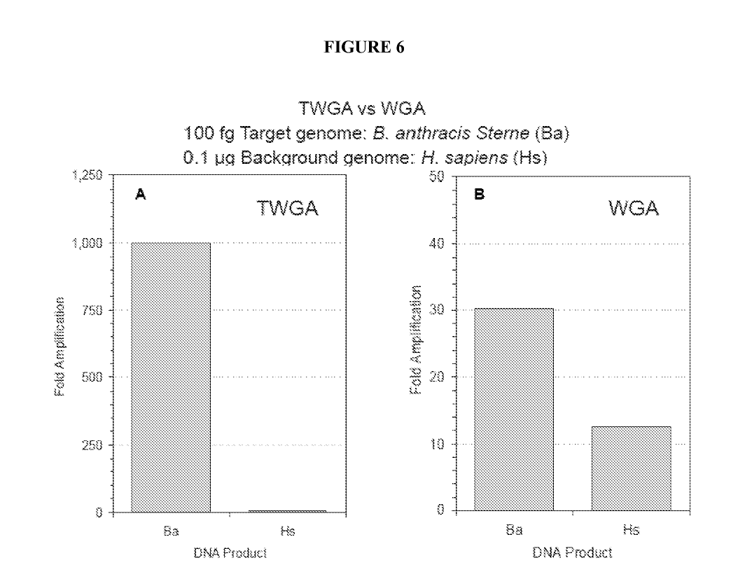

A set of algorithms was developed for the design of TWGA primer sets favoring amplification of target DNA from a DNA mixture as described in Example 2. As a test case, a TWGA primer set consisting of approximately 200 primers was designed for the preferential amplification of Bacillus anthracis genomic DNA from a mixture of background genomes. The primer set showed high representation of the Bacillus anthracis genome and under-representation in a panel of eukaryotic genomes selected from mammals, insects, plants, birds, and nematodes. The primer set was designed with consistent binding of the primers along the Bacillus anthracis genome, maintaining representation across the entire genome during amplification. To demonstrate the preferential amplification of target DNA from a DNA mixture, mixtures of Bacillus anthracis and human DNA were amplified using targeted whole genome amplification, and the resulting products were quantified by Quantitative Real-Time PCR-based detection of distinctive genomic sequences. As shown in FIG. 5A, 175-fold amplification of B. anthracis DNA was observed in the presence of a ten million-fold excess of human background DNA, with minimal amplification of the background DNA itself. A 3000-fold amplification of target DNA was observed when background was reduced slightly, to a million-fold excess relative to the target DNA levels, again with minimal amplification of background DNA (FIG. 5B). Results obtained from the targeted whole genome amplification reaction are contrasted with results of an unbiased whole genome amplification reaction in FIG. 6. Target genome was prepared in a million-fold excess of background DNA and amplified by targeted whole genome amplification or by unbiased whole genome amplification. In contrast to targeted whole genome amplification, unbiased whole genome amplification uses random priming which should result in similar amplification of both target DNA and background DNA. In FIG. 6A it can be seen that targeted whole genome amplification favored amplification of the target DNA. In contrast, whole genome amplification produced similar levels of amplification of both components of the DNA mixture (FIG. 6B).

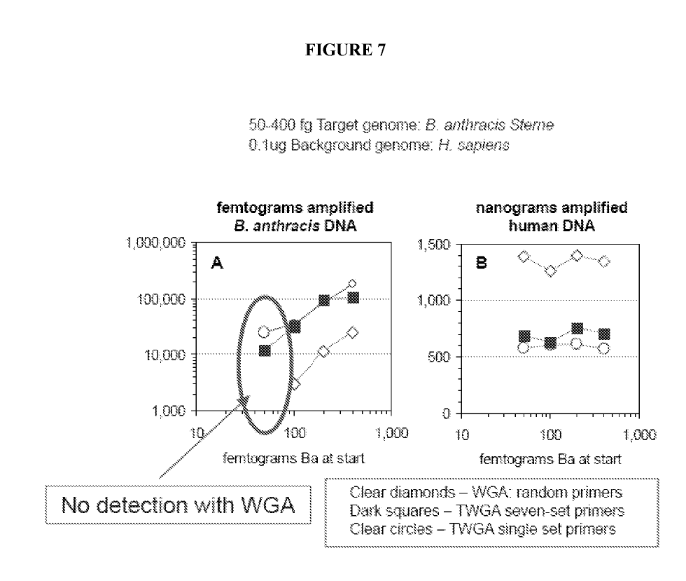

In FIG. 7, it is evident that targeted whole genome amplification increases the sensitivity of detection of target DNA from a mixture, in comparison to unbiased whole genome amplification. Reactions were prepared with human DNA present at 0.1 micrograms per reaction and with Bacillus anthracis genomic DNA incremented from 50 to 400 femtograms. Preferential amplification with targeted whole genome amplification primers was compared to unbiased amplification using random unbiased whole genome amplification primers. As shown above, targeted whole genome amplification gave higher yields of Bacillus anthracis DNA and lower yields of human DNA than unbiased whole genome amplification (FIGS. 7A and 7B). Significantly, targeted whole genome amplification gave detectable Bacillus anthracis product with 50 femtograms of starting material, whereas unbiased whole genome amplification did not. Targeted whole genome amplification primer sets were developed for six additional target organisms and a cocktail of the primer sets were run in the targeted whole genome amplification reactions. Similar results were obtained when targeted whole genome amplification was formulated with this pool of primer sets or with the Bacillus anthracis-specific targeted whole genome amplification primer set, indicating that targeted whole genome amplification can be multiplexed (targeted whole genome amplification seven-set primers vs. TWGA single-set primers, FIG. 7).

### Example 19

### Targeted Whole Genome Amplification Algorithm

This example demonstrates a method for generating a primer set for targeted whole genome amplification (TWGA) using ranking of oligonucleotides by combined hit ratios. The primer set includes 100-600 oligonucleotides that are 7-12 bases in length, and preferentially bind to a specific target genome or a plurality of target genomes over background genomes. The primer set minimizes that largest gap between primer binding sites on the target sequence. The primers are optimally no more than about 300 bases from one another. The target genome ideally is between 1 to 3 megbases in size. The background genomes might bet the human nuclear genome and human mitochondrial DNA. The TWGA primers include fewer oligonucleotides than primer sets described in the prior art. The TWGA primers are also superior because they are selected by considering the background genomes, and therefore are far less likely to unintentionally amplify background genome sequences as compared to primer sets known in the art.

In the first step, the number of times each oligonucleotide occurs in the target sequence is counted. The National Institutes of Health BLAST search tool, which is well-known in the art, can be used. For example, the target sequence might be.

(SEQ ID NO: 743)
ATCAGCGGATCTGACTGACTGACTGGCATGTAGCGGATTGCATG . . .

If each primer is to be seven bases-long, the number of times the following oligonucleotides appear in the target genome would be counted, as shown below.

```
(SEQ ID NO: 743)
ATCAGCGGATCTGACTGACTGACTGGCATGTAGCGGATTGCAT
G . . .
ATCAGCG
  TCAGCGG
    CAGCGGA
      AGCGGAT
        GCGGATC
          CGGATCT
```

This process is repeated for each oligonucleotide length, ranging from about 7 to 12 bases. For example, when such an analysis was performed on Burkholderia mallei with an oligonucleotide size of ten, the following results were obtained (SEQ ID NOS 744-757, respectively, in order of appearance).

| # TOTAL LENGTH (SINGLE STRAND) = 5835527 | |
|---|---|
| # TOTAL POSSIBLE COMBOS OF 10 = 1046676 | |
| # EXPECTED COUNT (DBL STRAND) = | |
| 11.1303844451904 | |
| cgcgcgcgcg | 3558 |
| cgccgcgcgc | 3397 |
| gcgcgcggcg | 3397 |
| cgcgcgcggc | 3131 |
| gccgcgcgcg | 3131 |
| cgcgccgcgc | 3114 |
| gcgcggcgcg | 3114 |
| gcgcgcgqgc | 2848 |
| cgcgcggcgc | 2829 |
| gcgccgcgcg | 2829 |
| cgcgcgccgc | 9730 |
| gcggcgcgcg | 2730 |
| cgcgagcgcg | 2636 |
| cgcgctcgcg | 2636 |
| . . . | |

The first three lines above show the length of the target genome, the number of different possibilities, and the expectation value of any oligonucleotide 10 nucleotides in length (i.e., the number of times one would expect to see in a genome of this size if A, G, T, and C were equally probable).

In the next step, the number of times each target oligonucleotide from above appears in the background genomes, such as a human nuclear genome and human mitochondrial genome, is counted. The frequency is divided by the background genome length to yield a hit ratio. The results may be as follows.

| | | | | BACKGROUND_HITS |
|---|---|---|---|---|
| OLIGO_ID | | SEQ | HITS | HIT_RATIO |
| 7_1 | cggcggc | 28940 | 228709 | 45.1101649710534 |
| 7_2 | gccgccg | 28940 | 228709 | 45.1101644710534 |
| 7_3 | cgccgcc | 22625 | 266046 | 30.3173332629497 |
| 7_4 | ggcggcg | 22625 | 266046 | 30.3173332629497 |
| 7_5 | cgccggc | 22433 | 149577 | 53.4664908832218 |
| 7_6 | gccggcg | 22433 | 149577 | 53.4664908832218 |
| 7_7 | cgccgcg | 20838 | 129075 | 57.5536728125335 |
| 7_8 | cgcggcg | 20238 | 129075 | 57.5536728125335 |

The hit ratio can be expressed as (# target hits/length of target genome)/(# background hits/length of background genome(s))

In the next step, the number of times each oligonucleotide appears in the human mitochondrial genome is counted. The frequencies from the target genome, the human nuclear genome, and the mitochondrial genome are combined to yield a combined hit ration, which is expressed as (# target hits/length of target genome)/(((# mitochondrial genome hits/length of mitochondrial genome)+(# human genome hits/length of human genome)/2) Such an analysis might yield the following results:

| OLIGO_ID | | SEQ | HITS | BACKGKROUND_HITS HIT_RATIO | TM | MITO_HITS | COMBINED_SCORE |
|---|---|---|---|---|---|---|---|
| 7_1 | cggcggc | 28940 | 228709 | 45.1101649710534 | 27.6336368226051 | 2 | 0.0259799794654524 |
| 7_2 | gccgccg | 28940 | 228709 | 45.1101649710534 | 27.6336368226051 | 2 | 0.0259799794654524 |
| 7_3 | cgccgcc | 22625 | 266046 | 30.3173332629497 | 27.6336368226051 | 2 | 0.0174604481301306 |
| 7_4 | ggcggcg | 22625 | 266046 | 30.3173332629497 | 27.6336368226051 | 2 | 0.0174604481301306 |
| 7_5 | cgccggc | 22433 | 149577 | 53.4664908832218 | 27.6336368226051 | 1 | 0.015396289684678 |
| 7_6 | gccggcg | 22433 | 149577 | 53.4664908832218 | 27.6336368226051 | 1 | 0.015396289684678 |
| 7_7 | cgccgcg | 20838 | 129075 | 57.5536728125335 | 29.7018255017817 | 0 | 0.0165732406298056 |
| 7_8 | cgcggcg | 20838 | 219075 | 57.5536728125335 | 29.7018255017817 | 0 | 0.0165732406298056 |
| 7_9 | cggcgcc | 20559 | 167828 | 43.6713594140391 | 27.6336368226051 | 0 | 0.0125756691594142 |
| 7_10 | ggcgccg | 20559 | 167828 | 43.6713594140391 | 27.6336368226051 | 0 | 0.0125756691594142 |
| 7_11 | ccgccgc | 19018 | 269199 | 25.1854980956924 | 27.6336368226051 | 3 | 0.0217573596917618 |

As result of the analyses above, a hit ratio has been calculated between every oligonucleotide in the target sequence as compared to the human and mitochondrial background genomes, as well as the combination (arithmetic mean) of the human and mitochondrial genomes.

In the next step, the oligonucleotides are ranked in descending order according to their combined hit ratios. Consequently, the oligonucleotides that preferentially bind to the target genome over the background genome are located at the top of the list.

In the next step is to generate primer sets that include oligonucleotides that preferentially bind to the target genome over the background genomes. The oligonucleotides are chosen from the ranked list one at a time. The goal is to pick oligonucleotides that bind to different areas of the target genome. In order to insure that a primer set does not include oligonucleotides that have very high hit ratios, but have lower frequencies in the target genome, a moving threshold is used. A pseudo-code that can be used to achieve this goal is:
Set target hit threshold to 0.
While the number of oligos in the set is less than the pre-determined size,
Grab the next oligo from the ranked list
Does it break up the largest remaining gap in coverage?
If yes,
Add oligo to set

### If no,

Discard oligo and continue
Is the set full?
If yes,
Increase target hit threshold and start a new set

Continue

This algorithm produces a series of sets of oligonucleotides, each with a different minimum number of target primer hits. The selection of primer sets requires a trade-off between sets that have a higher combined hit ratio and those with a higher maximum gap between adjacent primer sites. This is because the oligonucleotides with a high hit ratio tend to be longer (e.g., 11- and 12-mers) and infrequently appear in the background genomes. These oligonucleotides also infrequently appear in the target genome, but favor the target genome. A primer set might balance this trade off.

In order to perform this balance, for example, based on a search of a Borrelia genome, the following parameters might be assessed.

| [symbol] | [symbol] | [symbol] | [symbol] | [symbol] | [symbol] | [symbol] | [symbol] |
|---|---|---|---|---|---|---|---|
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |

[symbol]

indicates data missing or illegible when filed

The most important parameters are the average hit ratio and the maximum distance between primer sites. Ideally, a primer set has a high hit ratio and a small maximum distance between primer sites. These two parameters are at odds with each other, so a balance is struck between these two parameters. Ideally, the maximum distance between sites should be about 500 nucleotides, but if the hit ratio is poor where this threshold is reached, then a primer set with a higher maximum distance between sites might be selected.

### Example 20

### Detecting Borrelia

This example demonstrates that a primer set for targeted genome amplification (TGA) of selected parts of a genome can reliably detect Borrelia DNA, even when present only at trace amounts, and in the presence of overwhelming amounts of other background DNA, such as in a human blood sample. The method provides a quick, reliable, and accurate PCR test for Borrelia DNA.

The following three sets of TGA primers (designated groups BCT3511, 3514, and 3517) were generated for targeting Borrelia DNA according to the methods described herein. The following table discloses SEQ ID NOS 758-832, respectively, in order of appearance.

| Primer | |
|---|---|
| name | Sequence |
| 3517E-F1 | TCT GCT TCT CAA AAT GTA AG |
| 3517E-F2 | TAA CCA AAT GCA CAT GTT AT |
| 3517E-F3 | TTG CTG ATC AAG CTC A |
| 3517E-F4 | GCA ACT TAC AGA CGA AAT |
| 3517E-F5 | AGA CAG AGG TTC TAT ACA AA |
| 3517E-F6 | AGG TAA CGG CAC ATA TT |
| 3517E-F7 | TAA GAA TGA AGG AAT TGG C |
| 3517E-F8 | AAT TTA AAT GAA GTA GAA AAA GTC T |
| 3517E-F9 | GGC TAT TAA TTT TAT TCA GAC AA |
| 3517E- | TTG TCA CAA GCT TCT AGA |
| F10 | |
| 3517E- | TTT CTG GTA AGA TTA ATG CTC |
| F11 | |
| 3517E- | GAG CTT CTG ATG ATG C |
| F12 | |
| 3517-R1 | GCA ACA TTA GCT GCA TAA |
| 3517E-R2 | TCC CTC ACC AGA GA |
| 3517E-R3 | ACA CCC TCT TGA ACC |
| 3517E-R4 | TGA GAA GGT GCT GTA G |
| 3517E-R5 | TTG TAA CAT TAA CAG GAG AAT TA |
| 3517E-R6 | TTA GCA AGT GAT GTA TTA GC |
| 3517E-R7 | TGA TCA CTT ATC ATT CTA ATA GC |
| 3517E-R8 | CTA TTT TGG AAA GCA CCT |
| 3517E-R9 | GCA TAC TCA GTA CTA TTC TTT AT |
| 3517E- | TGA GCA TAA GAT GCT TTT AG |
| R10 | |
| 3517E- | TCT GTC ATT GTA GCA TCT |
| R11 | |
| 3517E- | TTA AAA TAC TAT TAG TTG TTG CTG |
| R12 | |
| 3517E- | ATT AGC CTG CGC AA |
| R13 | |
| 3514E-F1 | CCG AAA AAG ATG GGC |
| 3514E-F2 | AGG TTA AAA AGT CCG AAA C |
| 3514E-F3 | TCT CCC GAT CAA ATT AGA A |
| 3514E-F4 | AAA GAG ATA AAA GAT TTT GAA AGA A |
| 3514E-F5 | AAA GCT AGG TTT TTG GAG |
| 3514E-F6 | ACA GAA AAA GAA GAA GAA TTG A |
| 3514E-F7 | ATG ATG CTG GGA ATC AG |
| 3514E-F8 | GGG CTT GGA CTT GA |
| 3514E-F9 | GTC TTT TAA TGT GCT AAT GC |
| 3514E- | GCG TTC CTA CTA ATG TAT C |
| F10 | |
| 3514E- | GGC AGA GTT AAA ATA TAT GAA AAT |
| F11 | |
| 3514E- | CAC CCT TCA AGA ACT TTT A |
| F12 | |
| 3514E-R1 | ATA CCA AAT ATG AGC AAC TG |
| 3514E-R2 | AAG CCC AAT CCT AGA G |
| 3514E-R3 | TAG AAT TCA AAC TAG ATG CTG |
| 3514E-R4 | CGG TTC AAT TAC TAC ATA TTT TT |
| 3514E-R5 | GCC CGG TTC AAT TAC |
| 3514E-R6 | TCT TCA TTT AAA AGC TGC AT |
| 3514E-R7 | GCT CTC TAG CTT CTA TGT A |
| 3514E-R8 | AAG CAT TAA AAG ACA TAC CAT A |
| 3514E-R9 | GAA GAG TTT TAA TAG CCT CA |
| 3514E- | GAC GAA AGC TCA TCA AG |
| R10 | |
| 3514E- | CAG TTT TAT CAT CTT TAT CTA TCA TT |
| R11 | |
| 3514E- | AAA TTC TCA ATA ATT TCA AGA CG |
| R12 | |
| 3514E- | ATC CAC TCT GGC TTA TT |
| R13 | |
| 3511E-F1 | CGT GAA GCT GCA AG |
| 3511E-F2 | TGG AAA AGC AAT AAA AGC T |
| 3511E-F3 | TGT TGT ATA TGA ACA TTT ATT GG |
| 3511E-F4 | GCT TGG TAA TTC TGA GAT AA |
| 3511E-F5 | CCT CAA TTT GAA GGT CAA A |
| 3511E-F6 | ATT TTA AAG AGG GGC TTA C |
| 3511E-F7 | GCC ATG AAT GAA GCT TT |
| 3511E-F8 | CTC ATG TTA TGG GAT TTA GAA |
| 3511E-F9 | CTG ACA ACA TTC TTT CTT TTG |
| 3511E- | TGT TAA TGT GGG GCT TA |
| F10 | |
| 3511E- | GCT TTT CAA TCA GAA CCT |
| F11 | |
| 3511E- | GAG GGT GGG ATA AAA TCT |
| F12 | |
| 3511E-R1 | CCC ATT TTA GCA CTT CCT C |
| 3511E-R2 | GCA AAA TGG CCT GAA A |
| 3511E-R3 | GTT TTC TCA ACA TTA AGC ATT |
| 3511E-R4 | CAT TGG TGA TAA CCT TAT CTT |
| 3511E-R5 | TCC TGC ACC AAG AG |
| 3511E-R6 | CTT GTG ATA ACG AAG TTT TG |
| 3511E-R7 | CAT CAA CAT CGG CAT C |
| 3511E-R8 | AAG CTA AAA GCA AAG TTC TA |
| 3511E-R9 | ATA TCC ATT TTC AAT TAA ATC TCT C |
| 3511E- | TAA AGA GGA GGC ATG G |
| R10 | |
| 3511E- | AAA ATA ATA AAT ACG ATT GTC ATA CT |
| R11 | |
| 3511E- | TTG CGA TTT TTA GTT TCA ATA G |
| R12 | |
| 3511E- | CAA GCC CTT TAT ATC TCT G |
| R13 | |

Amplification was performed as follows.

Reaction vol 50
Borrelia TGA

| B buffer mix | stock | | | | |
|---|---|---|---|---|---|
| Reagent | conc | | final conc | # of reactions | 1 |
| 10X Buffer B | 10 | X | 1 x | 10X Buffer B | 5 |
| Sample | | | | Sample | 40.85 |
| dNTP | 25 | mM ea | 0.2 mM ea | dNTP | 0.4 |
| TGA primer 3517 | 200 | uM | 10 uM | TGA primer | 2.5 |
| | | | | 3517 | |
| BstE | 8 | U/uL | 0.2 U/uL | BstE | 1.25 |
| | | | | total volume | 50 |

The sample consisted of a simulant created by extracting 1 mL of human blood by methods known in the art, and spiking in around 200 genomes of B. burgdorferi B31.

The protocol was as follows:

All the components minus the BstE enzyme was mixed in a PCR tube. The tube was then put in a PCR machine for the following cycle.

95° C. for 3 min

Cool down and hold at 40° C.

The BstE enzyme was then added and the sample cycled at:

40° C. for 2 hours

80° C. for 20 min

4° C. hold.

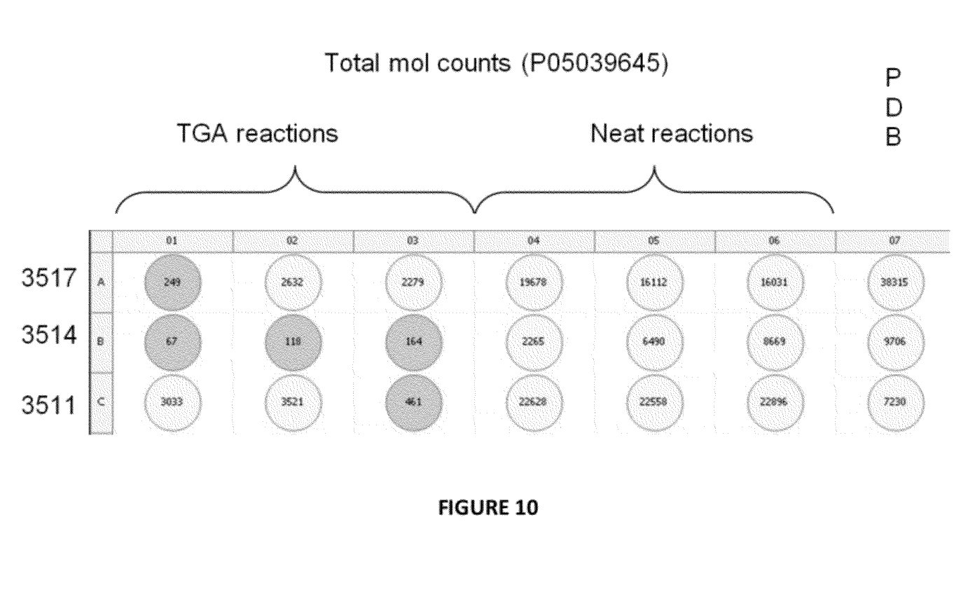

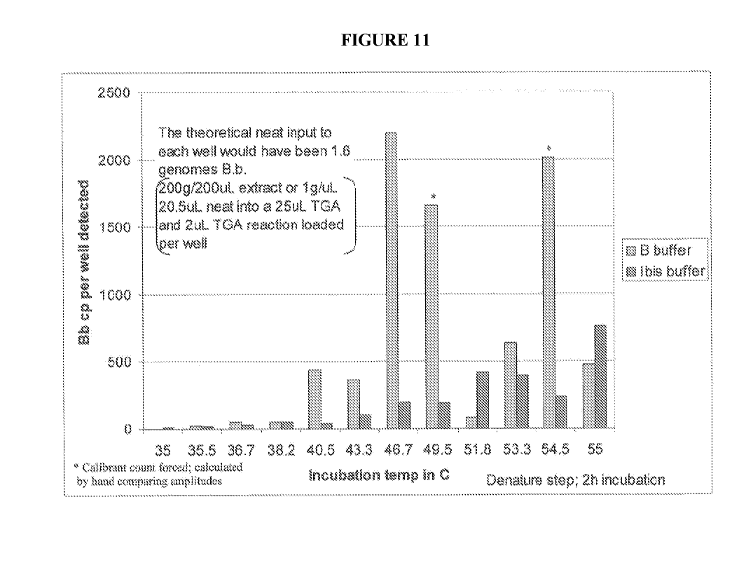

10 μL of sample were loaded into a TBS 5.0 plate for BCT3517, 3514, and 3511. It was observed that addition of 10 μL of the amplification reaction resulted in failed wells as determined by total mol count compared to the neat reactions. The results are shown in FIG. 10. An optimization experiment was performed to identify buffer and temperatures for TGA amplification. The same reactions as above were used, except for the buffer, and for the incubation temperatures (which ranged from 35° C. to 55° C.). The samples were run only for the PCR reaction for BCT3517. The results are shown in FIG. 11. The highest levels of Borrelia DNA were detected at incubation temperatures of around 47° C. using buffer B.

Incubation times were also tested to determine if shorter times would still provide an increase in Borrelia DNA. The following reaction conditions were used.

Reaction vol 200
Borrelia TGA

| | stock | | final | | | |
|---|---|---|---|---|---|---|
| Reagent | conc | | conc | | # of reactions | 1 |
| 10X | 10 | X | 1 | x | 10X Buffer B | 20 |
| Buffer B | | | | | | |
| Sample | | | | | Sample | 175.15 |
| dNTP | 25 | mM ea | 0.2 | mM ea | dNTP | 1.6 |
| TGA primer | 100 | uM | 1 | uM | TGA primer | 2 |
| mix | | | | | mix | |
| BstE | 8 | U/uL | 0.05 | U/uL | BstE | 1.25 |
| | | | | | total volume | 200 |

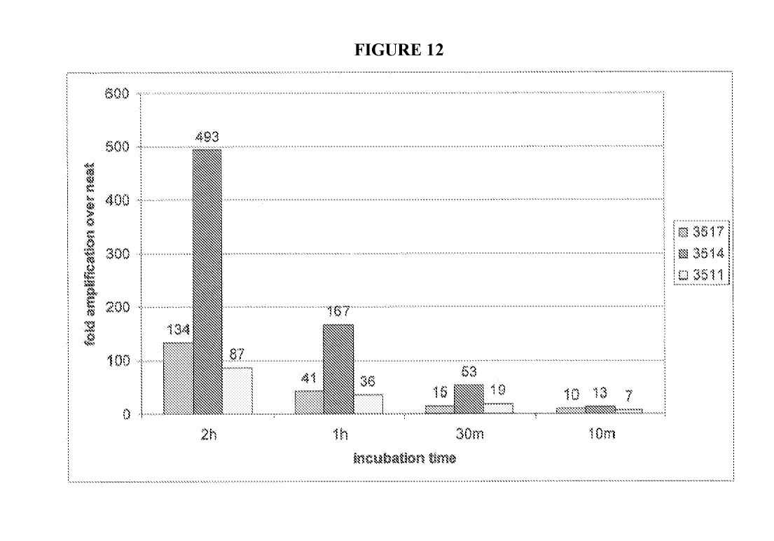

The results are shown in FIG. 12. It was found that even at short times, an increase in Borrelia DNA was observed.

A test of sensitivity was also performed to evaluate the limits of detecting using the TGA assay in conjunction with a TBS 5.0 Assay for detecting Borrelia DNA. Simulants were created by using 200 μL of human DNA extract and spiking in varying copy numbers of B. burgdorferi B31 genome and running 10 μL of the reaction on a TBS 5.0 plate.

The following reaction conditions were used.

Reaction vol 200
Borrelia TGA

| | stock | | final | | | |
|---|---|---|---|---|---|---|
| Reagent | conc | | conc | | # of reactions | 1 |
| 10X | 10 | X | 1 | x | 10X Buffer B | 20 |
| Buffer B | | | | | | |
| Sample | | | | | Sample | 175.15 |
| dNTP | 25 | mM ea | 0.2 | mM ea | dNTP | 1.6 |
| TGA primer | 100 | uM | 1 | uM | TGA primer | 2 |
| mix | | | | | mix | |
| BstE | 8 | U/uL | 0.05 | U/uL | BstE | 1.25 |
| | | | | | total volume | 200 |

The protocol was as follows:

All the components minus the BstE enzyme was mixed in a PCR tube. The tube was then put in a PCR machine for the following cycle.

95° C. for 3 min

Cool down and hold at 47° C.

The BstE enzyme was then added, briefly mixed, centrifuged, and the sample cycled at:

47° C. for 1 hours

80° C. for 20 min

4° C. hold.

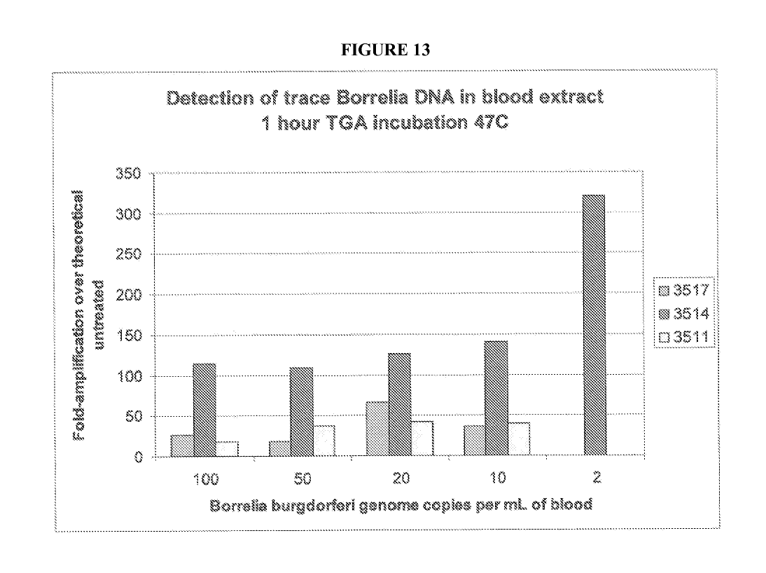

The results are shown in FIG. 13. Borrelia DNA was detected down to as few as two genomes in a total of 200 μL of human DNA extract, or equivalent to two genomes in 1 mL of human blood.

### Example 21

### TGA Primers for Detecting Whole Genomes

The method for TGA primer selection described herein was used to select primer sets for detecting Bacillus anthracis (BA), Yersinia pestis (YP), Brucella, Burkholderia, E. coli, Franciscella tularensis, and Rickettsia. The primers are described for each in Tables 18-24 below. An asterisk (*) indicates a phosphorothioate linkage.

TABLE 18
TGA Primers for Detecting Bacillus anthracis (SEQ ID NOS
833-1023, respectively, per column from left to right)

| | | | | |
|---|---|---|---|---|
| cgacttaccg*a*c | agaagcgat*g*a | aatcgcaa*t*t | gcttttttta*t*t | caattaat*a*c |
| tgtcggtaag*t*c | cttcttcttt*c*g | caccaatt*g*t | cttttaattc*t*t | attgaaac*g*a |
| tatatcrgcg*a*t | ttacgaaa*g*a | tgaagcga*t*t | catcaattg*t*t | attattat*c*g |
| aatcgcygat*a*t | tccgaaag*a*a | ttcacgaa*t*a | cgatataat*t*t | gcaattgt*t*g |
| tatatcgact*t*a | gcttcttt*c*g | gaaacgat*t*g | aagaagtaaa*a*g | ttcgtaaa*t*t |
| tatcggcgat*t*t | tgttctttc*g*t | tcaattgct*t*c | cgcttttta*t*t | tgaaacga*a*t |
| taacgaaaga*a*g | ttctttcg*c*a | cacctttta*c*a | atccgt*t*a | gctacttt*a*t |
| aaatcgttga*t*a | cttctttc*g*c | gaagaagta*g*c | cttcttta*a*t | gtattaaaa*g*a |
| ttgtcggta*a*g | gatacgaa*a*g | tcttttttc*g*c | tcattac*g*a | tgcttcttc*t*a |
| atcarcgatt*t*t | ttattatc*g*g | twacgat*t*g | atgtaac*g*a | taactcttc*t*t |
| tagaagaag*c*g | tacgaatg*a*t | tagaagc*g*a | caatcgt*a*t | tttattaga*t*g |
| ttctttcgtt*a*a | agcgaaaga*a*g | tagaagaa*g*t | taaagcg*t*t | cgatttttc*a*a |
| gattaaagtt*t*c | aagaagcga*a*g | caattgga*a*t | ttcaata*c*g | attaaagat*g*g |
| taaagtgaaa*c*t | tcgttaca*a*t | ttgtaatt*g*g | ttaacgg*a*a | taatam*c*g |
| ctttcgcttc*t*t | tttcgtat*a*t | aaataacg*a*t | gaagaagt*a*a | taatcg*y*a |
| gaagaagcga*a*a | caccaatt*a*c | ctttcgct*t*t | cgttaat*t*g | cgtaat*a*t |
| ctgattaaag*t*t | ctttttcg*t*a | tcgcttta*t*t | tattgatg*a*a | cttcgt*a*a |
| tacgaaaga*a*g | tgaaaaag*c*g | tttcgtta*t*a | taacagaa*g*a | atgaag*c*g |
| ttaacgaaa*g*a | tgcgaaaga*a*a | tttwtcgt*a*a | tatgtaa*c*g | catacg*a*a |
| cttctttcg*g*a | attgaaaaag*c*a | cgataaag*a*a | cgattga*a*g | atacgg*a*a |
| tctttcgc*t*a | gcaattgaaa*a*a | attacgat*a*a | ttagaaga*t*g | ttccgt*a*a |
| gcgaaagaa*g*c | catataa*c*g | atttatcg*t*a | taattgct*t*c | atacga*t*g |
| aacgaaagaa*g*a | aatcgttt*c*a | cttcttca*c*g | aacaccaa*t*t | tactcg*t*t |
| accgataa*t*a | tawtacga*a*a | atattatc*g*t | tgtaaaag*g*t | cggtaa*a*t |
| tcttctaa*c*g | aacgaaag*a*t | taaatcttc*t*a | tccaattg*a*a | tcgtat*t*g |
| aatcgc*t*a | cgtata*a*c | acaacga*t*t | tctacaat*t*a | aagcg*g*a |
| tattcg*c*t | taatgaag*a*a | cgttatg*a*a | cttccata*a*t | gatta*c*g |
| taccgt*t*a | ttcttta*c*g | ttgcaatt*g*c | tcaattgc*a*a | cgata*a*c |
| attaac*g*c | aaaacaat*t*g | attgcttc*t*t | tcctgtta*c*a | cggat*a*t |
| tattag*c*g | taacgga*a*a | tacaattt*c*a | gcaattgt*a*t | atatg*c*g |
| atcata*c*g | ctttttya*t*c | aaaaggta*t*t | gatgaatt*a*g | gttcg*t*a |
| gcgtaa*t*a | attatatg*a*a | attacaaa*a*g | cttttgtaa*t*a | cgcaa*t*a |
| aaaagaat*t*a | attacgt*t*a | tctttata*t*g | aaatggtga*a*g | cgatg*t*a |
| ctatcg*t*a | tcttcaat*a*t | cttctgca*a*t | tgaaacaat*t*g | gttgc*g*a |
| ttctttta*t*a | acgaagc*a*a | tctactaa*t*t | taaagataa*t*g | tcgca*t*a |
| tccgct*a*a | caacttct*t*t | aaaacgtt*t*a | tctttatat*t*c | |
| cttttttc*a*t | aaaaggtg*a*a | gttaattg*a*a | tgcaa*c*g | |
| aacgaat*a*a | cggttta*a*t | attacttg*t*a | tccgt*a*t | |
| atataaaa*g*a | attgtcg*t*t | cttctata*t*a | ccgct*t*t | |

TABLE 19
TGA Primers for Detecting Yersinia pestis (SEQ ID NOS
1024-1211, respectively, per column from left to right)

| | | | | |
|---|---|---|---|---|
| aacgggctac*c*g | agcgatta*c*c | tttatccg*c*a | ttgatcg*c*c | ccgtatt*g*a |
| gtatcccggt*a*g | ggcgattg*c*c | atggcgct*g*g | tgccggt*a*a | cgatatc*c*c |
| cttacggccc*g*t | tatccagc*g*c | ctccggtg*g*c | cagcggt*a*a | cgctgat*c*g |
| cccgtttacg*g*g | gcaatacc*g*t | csaatac*c*g | aacccgc*c*a | gcccgat*g*g |
| tgttagggcg*c*g | tcaggcgy*t*g | ggcggtw*t*t | gcgatga*c*c | gtaacgc*c*a |
| ccctaacagg*c*g | gccggtat*t*g | caatacc*g*g | cccgcca*g*t | ggtaacg*g*c |
| gtacttcggc*g*c | tgcgggta*t*t | ggcgata*g*c | agtatga*c*g | gtttacc*g*c |
| cttataggcg*c*a | ttacagttcc*a*t | tttatcgc*c*a | gccctga*c*g | taatgc*g*g |
| tgcgccgaag*t*a | caggcgtt*g*g | ttgccgcc*t*t | cggtaaa*g*c | cgatac*c*c |
| gcgcctataa*g*g | tatctgcc*g*t | gatatcg*c*c | gccgcta*t*t | taccgg*g*c |
| ccggcatagc*c*g | ggcaacgg*t*a | taccgcc*a*g | ccaatgc*g*g | ggcgatg*a*t |
| ttgccagacc*g*c | tggcaatc*g*c | gcggtta*t*c | ctgacgg*c*g | atgccga*t*g |
| agtgattcgg*g*t | ttatccag*c*g | ataaccg*c*c | cattacc*c*g | gctggcg*c*a |
| tgcggtctgg*c*a | aataccgc*c*a | tcgcggg*c*a | taccggt*a*t | atcgcca*c*c |
| tgccggagga*t*a | atgaatac*c*g | cgatagc*g*g | rccggtt*a*t | gcggctg*a*a |
| gttccatcgg*g*t | aacaggcg*a*t | ggcagata*g*c | ccgataa*c*c | tcatcgg*c*a |
| ctctcgatcc*c*g | gccgccag*t*t | tcatcaac*g*t | cgtttag*c*g | gttggcg*g*c |
| tactgaaccc*c*c | atcgctat*t*g | atcgccat*c*a | atttttac*c*g | gcgctgt*t*g |
| gggtcagtta*t*a | atcgccga*t*g | ttgatgac*g*t | cgccagc*a*a | aatggcg*g*t |
| atcctcaccg*t*t | cggcagat*t*g | cgccaat*a*a | attggcg*t*t | cgcttta*t*c |
| gtcaataacc*t*c | tcgccacc*g*g | taccggc*a*a | gccgctg*a*t | cacggta*t*t |
| gtgaggatag*g*c | attcgcca*c*c | ataccgg*t*g | mactggc*g*c | ttggccg*c*a |
| gctatgccg*g*a | atccagcg*c*c | atcaccc*g*c | gccgctt*t*a | ttgcccg*c*c |
| tagctggggg*t*t | accgttgc*c*g | tttdgcc*g*c | tcggtat*t*g | gataaac*c*g |
| ggggtttgtc*a*g | rataccc*g*c | tattrcc*g*c | attaccg*g*t | tcaccga*t*a |
| gtcatcggg*g*t | atcatcac*g*c | gcgatag*c*g | tatcggc*a*t | aaccggt*a*a |
| aaacccccag*c*t | gatgcgtt*g*a | tcaatatt*g*g | aatctgs*c*g | tgcggat*a*t |
| agcatcagac*t*g | cggtgctg*a*c | gtaatac*c*g | ccaatrg*c*g | gaaccgg*t*g |
| acgctttac*t*c | aaagtgcc*c*g | cgtaatg*c*g | cggtaat*a*t | atttacc*c*g |
| tgaggttatt*g*a | catcaccg*g*c | gctaacc*g*c | gcggtaa*t*g | ctggcga*a*t |
| atgcttctgg*a*g | aaagctat*c*g | gcgggta*a*c | gataacc*g*t | accggca*t*c |
| ccgcaata*c*c | cggccaat*a*a | tggcgat*c*a | ctggcgc*g*g | ttaccgt*c*a |
| taaccgcc*a*g | agccagcg*c*a | cgccagc*g*c | agataac*c*g | ggccgtt*g*g |
| cactggc*g*t | cgccttg*a*t | tatcsgt*c*a | gtaccc*g*c | tggcgat*g*g |
| gcgttga*t*a | attggcg*g*g | tgcgcca*t*a | cgtacc*g*g | tggctgg*c*g |
| cataacg*c*a | cagtaac*g*c | tgaactgg*c*t | gcgtac*c*g | cgaacgt*t*t |
| aatgccc*g*c | ttataac*g*g | atggcg*g*c | gccaata*a*c | |
| ggtgacc*g*c | cggtatc*c*a | tagcgg*g*t | aaaatac*c*g | |

TABLE 20
TGA Primers for Detecting Brucella (SEQ ID NOS 1212-1405,
respectively, per column from left to right)

| | | | | |
|---|---|---|---|---|
| tagagcggtt*c*c | cggccttg*c*g | tcgcgccg*g*g | cttcggcg*a*t | gatcgacg*g*c |
| cggaaccgct*c*t | attgcccg*c*g | gcctttgc*c*g | cgacgatc*a*g | gatatcga*g*c |
| gcgcatcccg*a*a | atcatgcg*c*g | cgcgcgct*t*c | gcggcggc*a*a | tcggcgga*a*a |
| ttagagcggt*t*c | gccgcgcg*c*a | gcctgccg*g*a | gaaagcgg*c*g | ggcgcaga*t*g |
| aaagtgcgaa*g*c | tgcggcgc*g*c | cgcaatgg*c*g | tgccgccg*a*c | gatgccga*c*g |
| gaaccgctc*t*a | caaggcgc*t*t | ctggcgct*c*g | cgcgcgcc*g*g | gccgccat*s*a |
| ccggaaccgc*t*c | tcggcggc*a*t | cgaaaagc*c*g | gcttccgc*c*g | cggcatgg*t*g |
| cacttttcgg*g*a | tgccgatg*c*g | cgcggcag*g*c | cacgcgcg*g*c | cggtgcgc*g*c |
| cgtttcacac*t*t | gctttcgg*c*g | gccaatgc*g*g | agcttgcg*c*a | ccggttct*g*g |
| sgcgcttgc*c*g | tgcgcgcc*g*a | tccggcgc*a*t | gcggcatg*a*a | catgcccg*c*c |
| gcgccgatc*t*g | gatcaggc*g*c | cttcggcc*c*g | gcgatttc*c*g | cgccatcg*a*g |
| gcgcttgcc*g*a | gcgcttcg*g*c | gcgccatg*g*c | ccttggcg*g*c | cgacgatt*t*c |
| gcggcaagc*g*c | ggcgcgca*t*g | cgcaagct*t*g | tgcggcgc*t*g | cggcagaa*g*c |
| cgccggaaa*g*c | tcagcgcg*c*c | gaccggca*a*t | atgccgat*g*g | tgccatgg*c*g |
| cggcaaggc*g*c | tcgcccgc*a*t | cgcaatcg*c*c | ggcgcagg*c*t | tcttgccg*a*t |
| cgatattgc*c*g | cggcwtcg*g*c | gcgccagc*g*g | cgcgcggg*c*a | atggcgcg*g*a |
| gcttcgcac*t*t | ccttgccg*r*a | atcgagcg*c*g | gcgctggc*g*a | accggcc*c*g |
| tgaaacggtt*t*t | aggcgcgc*a*a | tcggcatg*g*c | gccgggcg*a*a | ccgatac*g*c |
| gcgcgcaag*g*c | ggcgcggc*t*t | gccgcatc*g*c | gcgcgctt*c*t | tgcgcga*c*c |
| gcgccagcg*c*a | gcgcgcca*g*c | gcggcaat*c*a | ggcgaagc*g*c | attttccg*g*c |
| gccgatctg*a*t | gatgcgcc*g*c | gatgcggt*c*g | atcggcgg*c*g | cgccatca*t*g |
| gccgatgcg*c*t | cgcttcgg*c*a | gcgtctgg*c*g | gaaaaggc*c*g | ctcgatca*t*c |
| gccggaaag*c*g | gcgcaagc*c*g | gcggtcgc*c*a | aaaagccg*c*c | aatgccgc*c*a |
| tcggcaag*c*g | cgcgccga*a*a | ccatgccg*c*c | gccgcgcc*g*a | tcgatac*g*g |
| cgcctgat*c*g | catcggca*c*g | cgccatgg*g*c | gcatcgcc*g*c | ggcgttg*c*g |
| tcggccttg*c*c | atctggcg*c*g | gctcgacc*a*g | tgccgaaa*g*c | cggcccg*a*t |
| ttgcccgcg*c*c | ctgatccg*c*g | cgccgatc*c*g | cggcgcgc*t*c | cgatttca*t*c |
| gcgcgcct*t*s | gccttgcc*g*c | gcctttcg*g*c | cggcaagc*t*c | cgccgctt*c*c |
| agcgccagc*g*c | gcatccgc*g*c | acgccgga*a*a | cggccagt*t*c | tgaaaacg*g*c |
| gcgctgat*c*g | ttgcggat*c*g | tgccgcgc*c*c | cttcggcc*t*t | ccggcggg*c*g |
| cggcaaggg*c*g | cagcgtat*c*g | cgatgcgg*c*a | tgcgcggc*g*g | acgatgc*g*g |
| gcgccttg*c*g | ccgaaggc*g*c | gcgaaacg*c*c | gcaagcgc*a*t | attgccg*a*c |
| cggcaggc*c*g | ttgcgcgc*c*a | gccggtgc*c*g | aaggccgc*c*g | ggccgca*a*c |
| cgcctgccg*c*a | cgcgaagg*c*g | catttccg*g*c | caatggcg*g*c | cgcccgcc*g*c |
| gcttgcgc*g*c | gcggcagg*c*g | ttggcggc*a*a | cggcggca*c*g | cgcatgg*t*c |
| cgccttgc*c*g | ggcttccg*c*c | gccggaac*t*g | cctgcgcc*a*g | gcgcgcac*g*c |
| gcgcgsca*t*c | cgatatcg*a*c | tgcgcgcc*c*g | ttccggca*t*t | gcgaggcg*g*c |
| cgccaatg*c*g | cgggcggc*a*a | ccgatgcc*g*g | atcatcgg*c*g | atctgcc*g*c |
| gccatgcg*c*g | ttcggcct*g*c | cgaaatgc*g*c | cttcatgc*c*g | |

TABLE 21
TGA Primers for Detecting Burkhoderia (SEQ ID NOS 1406-1590,
respectively, per column from left to right)

| | | | | |
|---|---|---|---|---|
| cgacgctcgc*g*c | cgacgagcgc*g*c | cgcgcacgcg*g*c | ccgcgccgcg*c*a | cgcgttcct*c*g |
| gcgcgtcgmg*c*g | gcagcgcgtc*g*a | cgcgcgcatc*g*a | cgcttcgacg*c*g | cttcgacga*g*c |
| gcgctcgwcg*c*g | atgccgccga*a*c | cgcgtcgaac*g*c | cgcgcagga*c*g | tcgtcggg*c*a |
| agcgtccgcg*g*t | tcgcggcggg*c*g | atcaagcgcg*g*g | acgccgatg*c*g | catgtcgc*g*c |
| gaacttcgcg*c*g | cgcgcacgtc*g*a | cgcgctcgtg*c*t | gcgccgacgc*c*g | cgacggcac*c*g |
| gcgmgcgcgt*c*g | cgcggcgctt*g*a | ctcgccgcgc*g*c | tgcgcgtga*c*g | cgaggccga*t*g |
| agcgcgtcgc*g*c | cgcgagccgc*g*c | ccgatgcgcg*c*g | cgccgcgccg*t*y | gaacgcgg*t*c |
| cgcggcgagc*g*c | cgacgcccgc*c*g | cgccgcccgc*g*a | attcggcg*c*g | cgcgcgcgcg*t*t |
| cgagcgtcgc*g*c | gcgcatcggc*g*c | gctcgagcgc*g*c | gcgtcttcag*c*a | ccggctcg*c*g |
| gacgcgctcg*c*g | cggcgagcgc*g*t | cgcggtcgcg*c*t | ggcgggcgcg*c*g | tcgtgctcg*a*g |
| gcgtcgagcg*c*g | ctcgcgcagc*g*c | acagcgcgcc*g*a | cgatgtcgtc*g*a | caccgcgccg*c*c |
| tcgkcgcgct*c*g | aaccgccgaa*c*c | cgccgcgttc*g*c | acgagcggca*g*c | tcgccgcccg*c*c |
| cgcgatcggc*g*s | gcgcgctgcg*c*g | gttcggcgcg*c*g | cgggcttgc*c*g | cgtcgtga*t*g |
| cggcgcgctc*g*y | cgmcgatgcg*c*g | cgtcgcgcgg*c*a | cgtcgacgac*g*t | cgcgcaga*a*g |
| cgcgccgcgc*g*a | gtcggcgcgc*c*g | cgcgccgacg*c*t | cgtcgaaca*g*c | cggcatga*c*g |
| agtgacgcgt*c*g | gcgacgcgcg*c*c | gcggcgagcc*g*c | gccgcccgt*c*a | ccgcgccgcc*g*t |
| cgcgctcgcc*g*m | gccgcgcgct*c*g | ttcggcgcgg*g*c | attcgagcg*c*g | cgagcgaat*c*g |
| tcgcgcgcga*g*c | gcgckcgccg*a*a | cgcgcgaacg*c*g | cgtcagcac*g*a | acctgctcg*g*c |
| cgagcgcgtc*g*a | tccttgaccg*g*c | gagcgcgatc*g*c | tcgcatcgg*c*g | agcgggcgc*g*c |
| cgcgagcgcg*c*c | gcgcgccgtt*c*g | ggcgatcgcc*g*c | cgccgacg*c*a | aaagccctga*g*c |
| gatcggcgcg*c*c | atcgtcgcgg*g*c | cgacgccgcg*c*t | gctcgagat*c*g | tccgcgac*g*c |
| cacgacgctc*g*c | cggcgtcgcg*c*t | gacgccgagc*g*c | ccgctacg*c*g | atcgcccg*g*c |
| aaccgcggac*g*c | atcggcgtcg*c*g | ccgcgcacgc*g*a | cgccgaaat*c*g | gcacgggc*g*t |
| gcggcgctcg*c*g | tcgcgagcgc*g*t | ttcggcggcg*c*g | acgccgagc*c*g | ggcgtgac*c*g |
| gcgatcrcgc*c*g | cgatcggcgc*g*a | atcgcggcgc*g*c | cggcaagcc*g*a | cgccgaag*t*g |
| cgcgccggcg*c*g | gcggccgtcg*c*g | gcgcgcatcg*g*c | tggccgcggc*g*g | tacggcgc*g*g |
| gcgcggcgat*c*g | cgcgccgagc*a*s | cgatcaccgc*g*c | cgagcttgt*c*g | actgcgcg*a*g |
| cgtcgattcg*g*c | tcgagcggcg*c*g | cttcggcgag*c*g | gaacgccgg*c*g | cggcatcgg*c*t |
| tccggcttcg*c*g | agactccggc*t*g | catccggcgc*g*c | cgcttcgac*g*a | cgtcctcga*c*g |
| cccgacaagc*c*g | tcgcggccga*g*c | cgacgatcgc*g*g | cggtgcgcg*t*g | cgtgctga*c*g |
| gcgcgccgac*g*c | tcgcgcgcga*c*g | cggcgaacgc*g*g | tcgacgtcg*t*g | ggcatgcc*g*c |
| cgcgcgytcg*g*c | cggccgcgcg*c*g | cgcgcgcaag*c*g | acgtcgcg*a*a | cgcgaact*t*c |
| gcgctcgcga*a*g | cgtcgcgatc*c*a | cgcgtgctcg*a*c | cggcggcggg*c*c | gcggtccg*c*g |
| gmgcgcgccg*c*g | tcggcatcgc*g*c | gcctgccgcg*c*g | cgtcgcgc*t*t | acatgaagaa*g*c |
| cgcttcgcgc*g*c | ggccgcgagc*g*c | cgctcgtcgg*c*c | gcgctcacg*c*t | tgcgcacg*g*c |
| gcgwcgcccg*c*g | cggcacgctc*g*c | gagcgcttcg*g*c | gcttcgcg*a*a | gcgccgccgt*c*g |
| agcgcgacga*g*c | cgatcgcggc*g*g | cgggcctcgc*g*c | aggcccgc*g*c | acgccgcg*a*t |

TABLE 22
TGA Primers for Detecting E. coli (SEQ ID NOS 1591-1780,
respectively, per column from left to right)

| | | | | |
|---|---|---|---|---|
| cggataaggc*g*t | acgcgccag*c*g | aactggcg*c*g | gcgtttac*g*c | cgccagcg*g*c |
| cctgatgcga*c*g | cgcgctgg*c*g | ctggcgctg*a*t | acgctggc*g*g | ccgctggc*g*a |
| agcgtcgcat*c*a | gctggcgc*g*t | gcgccagc*g*c | caggcgctg*g*a | cgcgctgg*a*t |
| cgccttaccc*g*g | ttccgccag*c*g | ttaacgcc*g*c | gcrgcggt*a*a | ctgcgcca*g*y |
| aggcgttcac*g*c | cgctggcg*r*t | ctggcgct*g*g | cgatacgc*t*g | cgcgccag*c*a |
| gatgcggcgt*g*a | caatcgcca*g*c | gcgctggc*g*r | cgccagac*c*g | ggcgatac*c*g |
| tatcaggcct*a*c | cgccagcg*c*a | gaactggc*g*c | tcgccagcg*c*c | aacgcgcc*a*g |
| taggcctgat*a*a | cgccagtt*c*g | ggcaatcg*c*c | gcaactgg*c*g | ctggcgcg*c*g |
| gcctgatgc*g*c | ttacgccg*c*a | cgccagac*g*c | actggcgg*c*g | tgacgcca*g*a |
| ccagcgcc*g*c | atggcgtt*a*a | cggcatt*r*a | tgcggca*a*c | tggcatc*g*c |
| ctggctgg*c*g | attactgg*c*g | aacgcca*g*a | cggcagg*t*t | tctctgg*c*g |
| tgccgcca*g*y | aacgctgg*a*t | cgtctgg*c*a | aagcggg*c*g | tgcggtg*a*t |
| caggcgct*g*a | ggcgtta*a*s | tcaggcg*t*t | ctgacgg*t*t | acgtctg*g*c |
| ctttcgcc*a*g | cagcgcct*g*g | tccggca*g*t | aataccg*g*t | cgcgcag*c*t |
| cgctgacg*t*t | atcgccat*c*a | aaagcgc*c*g | ttcgcgc*t*g | gcgtggc*g*t |
| ccagcggc*g*t | ttccggca*g*c | ccggtaa*a*g | atcggcg*t*t | ccgcaac*c*g |
| gccgccag*a*c | acgccagc*c*a | acgccgc*a*g | acggtta*t*c | gatatcg*t*c |
| gattggcg*c*g | ctgcgtga*a*g | gcgcgta*a*t | gcgatac*c*a | cgttaat*g*g |
| cgctggaa*g*a | cgccatcg*c*g | gtgccgg*t*a | tggcgca*t*a | aaccggt*a*a |
| catccgcc*a*g | cgggcga*t*a | tcagcgc*g*a | cgcgtaa*a*c | cagcgtt*a*c |
| cagcgaac*t*g | atccggc*g*t | ccgggtt*a*a | aamgcgg*c*a | cagacgt*t*c |
| gattatcg*c*c | ccagccag*c*g | gactggc*g*c | cgctact*g*g | ccggaag*g*t |
| cgcgtctg*g*c | tggcgat*a*c | gaatacc*g*c | gcggtat*t*a | attgcctg*a*t |
| cgatctgg*c*g | ccagtac*g*c | tatcagg*c*g | ctgctgcc*g*g | tttttccg*c*c |
| cgccagca*g*g | aaaaagcc*g*c | tgctgac*g*g | ggtggcgg*c*a | gcggtt*t*c |
| tttcgcca*g*t | catcacgc*t*g | ctgccgc*g*c | cccgccgc*c*a | cgctaa*c*c |
| cgctgcgc*c*a | cgccagr*t*t | cattgcc*g*a | cgccg*t*a | catacc*g*g |
| gatcagcg*c*c | atacgct*g*g | aacggcg*a*a | cattgg*c*g | ggcgta*t*c |
| acgctgga*a*g | ttaacgg*c*g | tttaccg*t*c | ccggca*t*a | accgct*a*t |
| gcttccag*c*g | aacgccg*c*g | tattcag*c*g | cgcgtc*a*t | cccgcg*a*t |
| gccgcagg*c*g | acgcctg*a*t | gacgctg*a*t | ggttac*g*c | cgtagc*g*g |
| tgaccgcc*a*g | cggcgta*a*t | ttgcgcc*g*c | atgctgc*c*g | cgctgat*g*a |
| ggtgctgg*c*g | ggcgtac*t*g | tccgcca*g*g | atcgcct*t*c | tgcgtca*g*c |
| ggcggtga*t*g | atcaccat*c*g | accatcg*g*c | cttcgcc*a*t | ttttgcg*g*c |
| gcwgacgc*t*g | cgtcagca*a*a | cgcggcg*a*a | tgccggg*a*t | ccagcgt*g*t |
| ctgataag*c*g | cagcgttt*t*a | cagatcg*c*c | cgtcagc*a*c | cacgctg*g*t |
| cgctggcc*t*g | tgatggcg*g*c | atcaccg*g*a | tcagccc*g*g | gcttcag*c*g |
| gatgccgc*c*a | tcgctgcc*a*g | gaaacgc*c*g | attacgc*c*a | ttgcggc*a*g |

TABLE 23
TGA Primers for Detecting Franciscella tularensis (SEQ ID NOS
1781-1967, respectively, per column from left to right)

| | | | | |
|---|---|---|---|---|
| tagttaatcc*g*a | taggttctgt*g*c | ctatgttaaa*a*a | gctttagat*a*a | gatattac*t*a |
| cggattaact*a*t | gtagatataa*t*a | tatgataaag*a*t | agtattctc*t*a | ttgctata*g*c |
| tagcgactct*g*c | aaaaacttac*t*c | tctatataaa*a*t | tattgctat*a*g | tattgatg*a*t |
| gatatttgta*g*a | gtagaaatgt*t*a | ttaaaaaact*t*a | gctaaaga*t*a | tatagcta*t*a |
| ttactatagt*a*g | ataaaactct*a*t | gttattttat*a*t | gataagct*t*a | agatatta*t*c |
| atactccaac*c*t | tgatgtacaa*a*t | tataatcttt*t*t | gactatcaa*a*a | ttgataat*c*t |
| actatagtag*t*t | tatatccagc*a*a | atctatagc*t*a | gctaaaaaa*g*c | tagctata*a*t |
| gatagcatca*c*a | ataattatat*c*c | ttagcgat*a*t | gctatagat*t*t | gcwttagc*a*a |
| gctatttcaa*t*c | tagtttccat*a*a | agaaaaacta*a*a | agctaaag*a*t | tagtatta*t*c |
| tgaccatcct*c*t | ttattggcta*t*t | aatttttaat*c*a | tatctaaa*g*m | ttaccaat*a*g |
| tagacttggt*t*t | ctttttctac*a*a | tatmgcta*a*a | tagyttta*g*c | ctttagca*c*c |
| tgctaaaacc*t*c | taatgcttct*t*t | ttttagtttt*t*c | gctatagt*t*g | gcaactat*t*g |
| aacctttcat*g*t | actccaaaat*a*t | gttttgaaaa*a*t | ataactatc*a*a | taatgctga*t*a |
| gatagaaagc*t*t | ctttgtgttt*g*a | aaagtagct*a*t | tttagcta*a*a | tttgataa*t*m |
| atagatgaga*t*g | aaatgtactt*c*t | tatcaaaag*g*t | attagcta*t*a | takttgat*a*a |
| taatgctagt*t*t | aatcttttta*t*c | ttgcttggt*t*a | tctttagc*t*a | attgctaa*a*g |
| ttaagctt*a*t | cttattga*t*a | tttgataa*a*g | ctactaat*a*t | atagatga*t*a |
| tgattttga*t*a | tatagata*t*c | tctttatc*a*a | cataacta*t*a | tatcaaaa*g*t |
| taatatca*g*c | ttatctat*a*g | ctaatatt*a*t | ttatcaaa*a*t | atatcttg*a*t |
| cagctatt*g*c | atataact*c*t | tatcgcc*a*a | tttttgat*t*a | ttgctaaa*a*c |
| agtatcac*c*a | tggtattg*g*t | tatcaaac*t*t | taccaac*t*a | aaagatgg*t*g |
| gttggtga*t*a | cttttttat*c*a | tgttgatg*g*t | taaatcta*a*a | tattatca*g*t |
| ctaaagct*t*g | tacttcttt*t*a | aytatcag*c*a | ttatcata*a*a | tgataagt*t*t |
| gcaatagc*t*a | agcattag*c*t | aatttagc*t*a | tttgatak*t*a | tatatact*c*t |
| ttgctaaag*c*a | tactaata*t*y | tagtagta*t*t | ttggtaaa*a*a | tcaccatt*a*t |
| gataaaacc*a*t | aagattta*g*c | aactatca*a*t | tttatagc*t*t | aatatcta*t*c |
| atattgcta*a*t | agctataa*c*a | aaagttgc*t*a | ataatatc*t*a | atatcawt*a*c |
| ttttttag*c*t | ggtgatat*t*g | tttagctg*t*a | tgatgata*a*t | gatttatc*t*a |
| tgatattg*a*t | accagcta*a*a | caatatct*t*c | gctaataa*a*a | agcaactt*t*a |
| tgataaag*t*t | accataac*c*a | ataagctt*t*g | atatttta*g*c | ttagcttt*g*a |
| tttaccaa*t*a | gcgatatt*t*t | aamactat*c*a | ttatctaa*t*a | ttgatggt*g*a |
| agctaaag*c*a | gatggttt*a*g | cttgatga*t*a | atataact*a*a | taaatcaa*g*c |
| tttagata*a*g | ttatctaaa*a*g | atacttat*c*t | tcaaagat*a*t | tttttcga*t*a |
| agtattat*c*a | caatattat*c*a | atactatc*a*a | taaaccaa*t*a | ctcatcta*a*a |
| tgatttag*m*t | ttttgata*a*a | cttctaaa*g*c | agcttctt*t*a | tctagaga*a*t |
| tattatca*a*c | atcatcaa*a*a | ctttatca*a*g | attaaagc*t*a | |
| aatwgctg*a*t | caactat*a*g | gtatcaaa*g*a | gctgctga*t*a | |
| agtataga*t*a | aatatcat*c*a | tagaaatat*t*g | atataagt*t*a | |

TABLE 24
TGA Primers for Detecting Rickettsia (SEQ ID NOS 1968-2163,
respectively, per column from left to right)

| | | | | |
|---|---|---|---|---|
| taatatac*c*g | agtagtat*t*a | tactaaat*t*a | atattact*a*t | gcaaaagat*a*a |
| attatcgg*t*a | gctataat*a*t | taaatcta*t*a | tattatat*c*t | ataaacctt*t*a |
| taataccg*a*t | tactatat*c*a | atctatat*t*a | tatatcat*t*a | ctactattt*t*a |
| taatattagt*a*t | ttgataaatt*a*t | tgatatag*a*a | atctaata*t*t | attaatat*t*a |
| aatattac*c*g | aaagtaatat*t*a | ataatagg*t*a | tattattg*c*a | ttttagta*t*t |
| cggtaaaa*c*t | ttagtaaaaa*a*t | ctatagta*t*t | ttaatata*t*c | aactttaa*t*a |
| tctaatatat*t*a | tagttaaag*a*t | tactaatg*c*t | tctaatat*t*a | taatagta*a*a |
| aatactaata*t*t | gcattatta*c*t | ttacgtaa*a*t | tatcaata*t*a | aattatct*a*t |
| atattagtat*t*t | tttattacta*a*a | ataaataag*c*t | ttggtaat*a*a | tattaata*c*t |
| tttatctata*a*t | gcttataa*t*a | acgtaata*t*t | tactaaag*a*a | attattaa*g*t |
| tattgcga*t*a | ttaccgc*t*a | actattat*a*g | tattatc*g*g | attaataa*t*c |
| cggtaata*a*t | atagtatta*t*t | tattagtta*t*a | gctataat*t*a | atcggta*a*t |
| tagtaatac*t*a | tagtaata*t*c | actataga*a*g | taaagtag*t*a | aggtataa*a*a |
| gataatact*a*t | gcaagtaa*t*a | ctttatcta*t*t | attaatac*c*a | atacttaa*t*a |
| aaaaaaggtg*a*t | atattagta*a*t | aattagctt*t*a | ttatatag*c*t | tttttagc*t*a |
| tattacgt*a*a | wtaaagcta*a*t | ctattttag*t*a | taataaattt*t*t | ttttagata*t*t |
| ttattagtaa*t*a | tagtattttt*a*a | gataaaata*g*t | aatttagt*a*g | aatacaag*a*t |
| tactaatat*t*a | atattgataa*a*a | taataaattt*g*a | agctataa*t*a | atagtttt*a*g |
| ctatatta*c*c | tatctaaaaa*a*t | ttagctaaa*t*t | cattacta*t*a | aggtaaat*t*a |
| ttttatctaa*a*t | tagtattat*t*g | tattacttg*a*t | gcaataat*a*g | ataatgct*a*a |
| cggtaaaa*t*a | ttgctaaaaa*t*a | tctttagtt*a*a | tattatag*g*t | cttttact*a*t |
| tagtattat*y*a | aataatattg*a*t | taataata*t*t | attgaagt*a*g | ataagctt*t*a |
| aataatacta*t*t | taactaaag*a*t | ttttacta*t*a | taatctag*a*t | aattaatag*a*a |
| atctattac*t*a | attataggt*a*a | tttaatag*a*t | attactag*a*t | atttagtaa*a*t |
| gcaataatt*t*t | attgattt*a*c | | | |
| gtactaaa*a*t | taaagaatt*a*t | | | |
| atcattac*t*a | acctttat*t*a | | | |
| gtaatatt*g*a | tactatca*a*a | | | |
| attctata*g*a | caataata*t*c | | | |
| aaactact*a*t | ctcttaat*a*t | | | |
| tagtgata*t*a | aaactacc*t*a | | | |
| ataatgac*t*a | atattatga*t*t | | | |
| tacttaaa*t*c | ggtaataaa*a*a | | | |
| atagtatc*a*a | attctaaat*t*a | | | |
| atatttat*c*g | tattaaaa*a*t | | | |
| aatagtag*c*a | ttattaat*t*t | | | |
| tcctacta*a*a | ataaaatt*a*a | | | |
| tagtatta*g*a | aaataatt*a*t | | | |
| gttaattat*a*t | atataaat*t*t | | | |
| ctataaaat*c*a | taaatttt*c*t | | | |
| tcataatat*t*a | caataaaa*a*t | | | |
| agctttaat*a*a | ataattat*a*a | | | |
| ttagaaaaat*t*a | taattctt*t*t | | | |
| ttagtatat*a*a | aagatttt*t*a | | | |
| tttagataa*a*g | aaaaattg*a*t | | | |
| aaaatatt*a*t | tgccga*t*a | | | |
| taatttaa*t*a | aagaataa*t*a | | | |
| aatttctt*t*a | tgatttaa*a*t | | | |
| tttatctt*t*a | gataatc*t*a | | | |
| tttattac*t*t | ttgcgg*t*a | | | |
| ttttatta*t*c | | | | |
| atcaatat*t*t | | | | |
| agtaaaat*t*w | | | | |
| attacttt*t*a | | | | |
| tgaatata*a*a | | | | |
| agcaaatt*t*t | | | | |
| tagtac*c*g | | | | |
| aaattagt*a*t | | | | |
| atagattt*a*a | | | | |
| caagatat*t*t | | | | |
| tgcattat*t*a | | | | |
| tgaagatw*t*a | | | | |
| attatata*g*t | | | | |
| agattata*t*a | | | | |
| atacggt*a*t | | | | |
| tattttatc*a*a | | | | |
| atttacta*a*t | | | | |
| tcttttat*a*g | | | | |
| atagtaaaa*a*a | | | | |
| gctaaata*t*t | | | | |
| tataatta*a*c | | | | |
| atttttttg*a*t | | | | |
| tacaagta*t*t | | | | |
| tcatgatt*t*a | | | | |

### Example 22

### Amplification of K. pneumoniae Target Regions

To determine whether targeted genome amplification (TGA) could be used to amplify trace amounts of pathogen target DNA, 40 μl of human DNA (extracted from 200 μl of blood sample) was spiked with 20 copies of Klebsiella pneumoniae genome. The DNA samples were suspended in a buffer solution containing 50 mM Tris pH 7.6, 12 mM MgCl₂, 10 mM (NH₄)₂SO₄, 6.6% betaine, 21.6% trehalose, 2.5% DMSO, and 1.1% Tween-40. Primers to 16S and 23S regions as described herein were added. The final sample volume was 160 μl. The samples were incubated at 95° C. for 3 min, then cooled to 37° C., whereupon 32 U of Bst polymerase lacking exonuclease activity was added per reaction. Samples were incubated at 50° C. for 2 hours, then subjected to an enzyme-denaturing incubation at 80° C. for 10 minutes. Samples were held at 4° C. until further analysis.

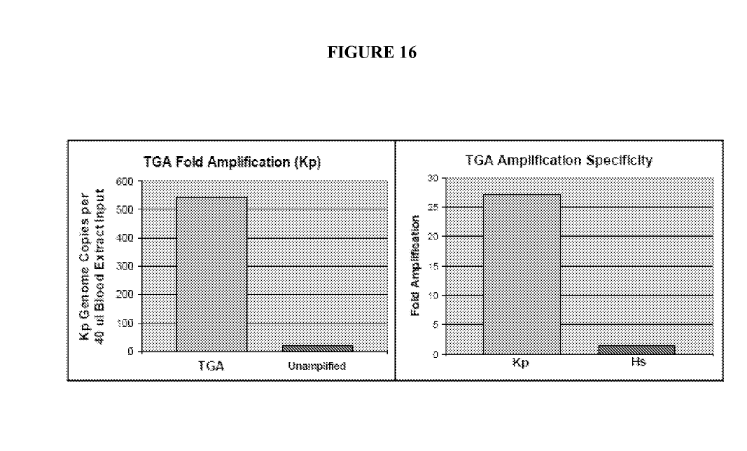

FIG. 16 shows the results of analysis of K. pneumoniae TGA reactions as described above using quantitative real-time PCR (qPCR) to quantify K. pneumoniae 16S (Kp) copy number and human (Hs) Alu copy number in comparison to unamplified controls. The results indicate that the TGA reaction permitted a greater than 25-fold amplification of Kp 16S region despite the presence of a 6,000,000-fold excess of non-target human (Hs) DNA.

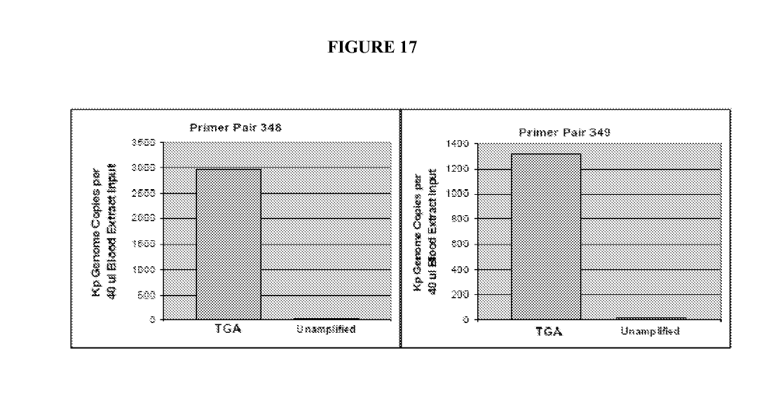

FIG. 17 shows the results of analysis of K. pneumoniae TGA reactions as described above using ESI-MS (electrospray ionizing mass spectrometry) with the Ibis T5000™ Biosensor system, where a calibrated Kp target DNA quantification was performed using a BCA plate. Results indicate that quantitation with two primer pairs referred to as 348 and 349, each directed to the 16S region, showed a 149-fold (for primer pair 348) and 66-fold (for primer pair 349) amplification of Kp genome in the TGA samples as compared to unamplified controls.

Table 25 shows that the limit of detection of TGA-amplified samples greatly exceeded that of the limit of detection of unamplified samples. Using the T5000 Biosensor system and primer pairs 348 or 349, signal was readily detected with as little as 1 μA of TGA reaction. In contrast, 10 μl of negative control (unamplified Kp-spiked human blood extract DNA) did not yield any detectable signal using the T5000 Biosensor assay.

TABLE 25
Limit of detection of 16S target region for K. pneumoniae-spiked
human DNA samples with and without TGA amplification.

| | | | | | 20 Kp/40 ul |
|---|---|---|---|---|---|
| | | Bst TGA Reaction | | | Blood Extract |
| | Primer | 10 ul/well | 5 ul/well | 1 ul/well | 10 ul/well |
| 16s | 348 | 233 | 163 | 93 | ND |
| 16s | 349 | ND | 155 | 41 | ND |

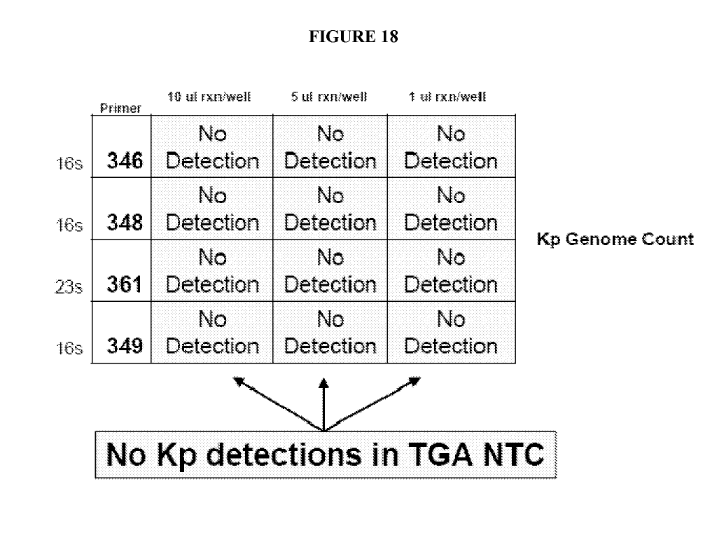

To determine whether any of the reagents used to perform TGA were contaminated with K. pneumoniae target DNA, no-template-controls (NTC) were analyzed using the T5000 Biosensor system and primer sets 346, 348, or 349 (for 16S target DNA) or primer set 361 (for 23S target DNA). FIG. 18 shows that no signal was detected using as much as 10 μl reaction per well.

### Example 23

### Additional Methods for Detecting Borrelia

Additional primers were developed for amplification of B. burgdorferi B31target regions, including primers longer than those used in set E (Example 20). New primers were developed on either side of each of one of the Spirochete targets. Parameters that were tested included 1) using the longer primers with fewer on each side, as well as 2) longer primers with the full 25 primers on each side of the target sequence. The new primers were compared to the original primers described in Example 20 (set E). The annealing temperatures were also varied to determine the optimal conditions. Table 25 includes additional primer sets, referred to as Primer Set “E2”.

TABLE 25
Additional primers used for TGA amplification
of B. burgdorferi target region 3511.
These primers referred to as the “E2 set”.

| | |
|---|---|
| Primer name | Sequence |

Set 3511E2 (SEQ ID NOS 2164-2188, respectively,
in order of appearance)

| | |
|---|---|
| 3511E2-F1 | CGT GAA GCT GCA AGAAAA |
| 3511E2-F2 | TGG AAA AGC AAT AAA AGC TGCTG |
| 3511E2-F3 | TGT TGT ATA TGA ACA TTT ATT GGAAAT |
| 3511E2-F4 | GCT TGG TAA TTC TGA GAT AAGAAA |
| 3511E2-F5 | CCT CAA TTT GAA GGT CAA ACAAA |
| 3511E2-F6 | ATT TTA AAG AGG GGC TTA CAGCT |
| 3511E2-F7 | GCC ATG AAT GAA GCT TTTAAA |
| 3511E2-F8 | CTC ATG TTA TGG GAT TTA GAA GTGG |
| 3511E2-F9 | CTG ACA ACA TTC TTT CTT TTG TTAA |
| 3511E2-F10 | TGT TAA TGT GGG GCT TAAATG |
| 3511E2-F11 | GCT TTT CAA TCA GAA CCT TATT |
| 3511E2-F12 | GAG GGT GGG ATA AAA TCT TTTT |
| 3511E2-R1 | TACCC ATT TTA GCA CTT CCT CCA |
| 3511E2-R2 | TGGCA AAA TGG CCT GAA AAA |
| 3511E2-R3 | TTGTT TTC TCA ACA TTA AGC ATT TT |
| 3511E2-R4 | ATCAT TGG TGA TAA CCT TAT CTT CT |
| 3511E2-R5 | ACTCC TGC ACC AAG AGAT |
| 3511E2-R6 | ATCTT GTG ATA ACG AAG TTT TGTA |
| 3511E2-R7 | TCCAT CAA CAT CGG CAT CTG |
| 3511E2-R8 | AAAAG CTA AAA GCA AAG TTC TAAT |
| 3511E2-R9 | ATATA TCC ATT TTC AAT TAA ATC TCT |
| | CAT |
| 3511E2-R10 | TATAA AGA GGA GGC ATG GCT |
| 3511E2-R11 | TAAAA ATA ATA AAT ACG ATT GTC ATA |
| | CTTT |
| 3511E2-R12 | TATTG CGA TTT TTA GTT TCA ATA GAA |
| 3511E2-R13 | CCCAA GCC CTT TAT ATC TCT GAA |

Set 3511EL (SEQ ID NOS 2189-2211, respectively,
in order of appearance)

| | |
|---|---|
| 3511EL-F13 | TGG TAA AGA AAA ATC TTC AAA ATT |
| 3511EL-F14 | CGA TAA AAT ATA CAT TTC AAT TGA AG |
| 3511EL-F15 | GGC TTA AAG AGC TTG C |
| 3511EL-F16 | AGA TTA TAA TTT CGA TGT TCT TGA |
| 3511EL-F17 | CGG ATT CTG AAA TTT TTG AAA |
| 3511EL-F18 | GGG GAC TAA GGT TAC TT |
| 3511EL-F19 | AGA AGT TGT GGG GGA ATC TTC |
| 3511EL-F20 | TGT GGG GGA ATC TTC |
| 3511EL-F21 | CTT TTT CAA AAG GTA TTC CG |
| 3511EL-F22 | GGT TTA TGT TAA TAG AGA TGG AA |
| 3511EL-F23 | GGT TGT AAA TGC TCT ATC TT |
| 3511EL-F24 | TGG TAA GTT TAA TAA AGG CAC |
| 3511EL-R14 | AGC TGC GTT GGA TT |
| 3511EL-R15 | CTA GCA GGA TCC ATA GTT |
| 3511EL-R16 | ATC ATC TAT ATT CAT CAA TCT CAT |
| 3511EL-R17 | GAG TAA CAA AAA TTT TTT CAG C |
| 3511EL-R18 | TTT CTG GGC TCA ACT AA |
| 3511EL-R19 | GAT TAA TTA CAT TAA GTG CAT TCT |
| 3511EL-R20 | CCA TTA ACG CTC CAA TT |
| 3511EL-R21 | TCC TAA CAT TTA ATA TTT GTT CTT |
| | TAT |
| 3511EL-R22 | GCA TAA TTT AAA TAA GAA GTT TTT |
| | ATT TC |
| 3511EL-R23 | GAA GAG CTC TAG AAA CAA TAA |
| 3511EL-R24 | TGG TTT AAG ACC ATC TCT T |

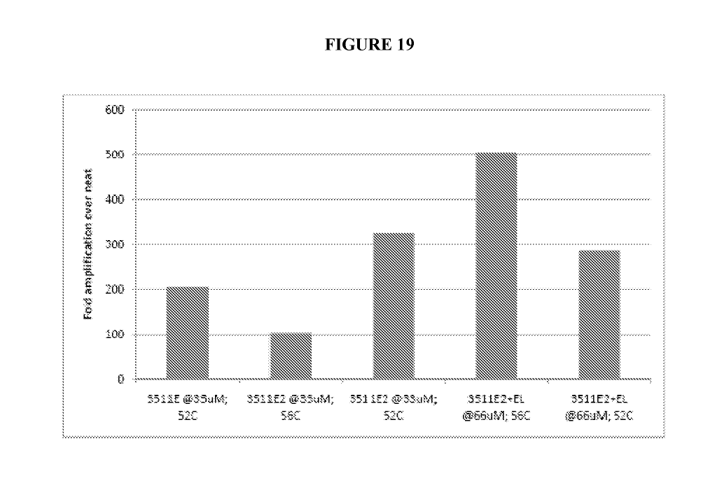

To test the new primer sets, human DNA was extracted from 1 ml blood to result in 200 μl DNA extract. The equivalent of 50 copies of B. burgdorferi B31 genome was added to each reaction. Amplification reactions were set up in which 225 μl total reaction volume included 1× PCR buffer, 197.04 μl sample, 1.8 μl dNTPs, 2.25 μl primer mix (at concentrations of 33 or 66 μM as detailed in FIG. 19), and 2.4 μl Bst polymerase. Samples were denatured at 95° C. for 10 minutes, held at annealing (primer extension) temperatures as indicated in FIG. 19 for an incubation time of 4 hours, and subjected to a polymerase inactivation step at 80° C. for 20 minutes and temperature hold at 4° C. Two microliters of each sample was analyzed per well using a TBS 5.0 plate for each of the indicated primers. Results shown in FIG. 19 indicate that the most fold amplification occurred at 56° C. using both the longer primers and the full set of 24 primers on each side of the target sequence.

An additional primer set, designated as “Set E3”, was designed as indicated in Table 26 below.

TABLE 26
Additional primers used for TGA amplification
of B. burgdorferi target regions 3517 (SEQ ID
NOS 2262-2311, respectively, in order of
appearance), 3514 (SEQ ID NOS 2212-2261,
respectively, in order of appearance), and
3511 (SEQ ID NOS 2312-2361, respectively,
in order of appearance). These
primers are referred to as the “E3 set”.

| Primer | Sequence |
|---|---|
| 3514E3-F1 | CCG AAA AAG ATG GGC TTTT |
| 3514E3-F2 | AGG TTA AAA AGT CCG AAA CTATT |
| 3514E3-F3 | TCT CCC GAT CAA ATT AGA AATTG |
| 3514E3-F4 | AAA GAG ATA AAA GAT TTT GAA AGA |
| | ATAAA |
| 3514E3-F5 | AAA GCT AGG TTT TTG GAG TTTT |
| 3514E3-F6 | ACA GAA AAA GAA GAA GAA TTG ATTAA |
| 3514E3-F7 | ATG ATG CTG GGA ATC AGGTTC |
| 3514E3-F8 | GGG CTT GGA CTT GATTTG |
| 3514E3-F9 | GTC TTT TAA TGT GCT AAT GCAAGA |
| 3514E3-F10 | GCG TTC CTA CTA ATG TAT CAGGG |
| 3514E3-F11 | GGC AGA GTT AAA ATA TAT GAA AAT ATAG |
| 3514E3-F12 | CAC CCT TCA AGA ACT TTT AACAG |
| 3514E3-F13 | GGC TCT TGA AGC TTA TGG |
| 3514E3-F14 | AGA CTT GGA GAA ATG GAG G |
| 3514E3-F15 | GAG GAA AGG CTC AAT TTG G |
| 3514E3-F16 | TCT TGT TTC TCA GCA ACC T |
| 3514E3-F17 | CGC AAG ATC AAC AGG C |
| 3514E3-F18 | ACT ACA CCA TCT TGT TGA TGA TA |
| 3514E3-F19 | GTA ATG GTT GGG GTG ATT TAC |
| 3514E3-F20 | GGA GAG CCG TTC GAA A |
| 3514E3-F21 | CCA ACT TCT AAA GAA ATT TTA TAT GAT |
| | GG |
| 3514E3-F22 | AGG AAA AAT TAA AAA CTG CTG GA |
| 3514E3-F23 | TGT TTT TGA ATC TGC TAC AAA TGA |
| 3514E3-F24 | CTG GTA AAT ATC TTG GTG AAT CTT ATA |
| | A |
| 3514E3-F25 | GGA CAG TTA ATG GAA TCT CAA T |
| 3514E3-R1 | ATA CCA AAT ATG AGC AAC TGGGGC |
| 3514E3-R2 | AAG CCC AAT CCT AGA GGGTA |
| 3514E3-R3 | TAG AAT TCA AAC TAG ATG CTG TAAT |
| 3514E3-R4 | CGG TTC AAT TAC TAC ATA TTT TTCATA |
| 3514E3-R5 | GCC CGG TTC AAT TAC TACA |
| 3514E3-R6 | TCT TCA TTT AAA AGC TGC ATTTTT |
| 3514E3-R7 | GCT CTC TAG CTT CTA TGT ACTCA |
| 3514E3-R8 | AAG CAT TAA AAG ACA TAC CAT ATCGC |
| 3514E3-R9 | GAA GAG TTT TAA TAG CCT CAGCCC |
| 3514E3-R10 | GAC GAA AGC TCA TCA AGATCA |
| 3514E3-R11 | CAG TTT TAT CAT CTT TAT CTA TCA |
| | TTTGAA |
| 3514E3-R12 | AAA TTC TCA ATA ATT TCA AGA CGTCTT |
| 3514E3-R13 | ATC CAC TCT GGC TTA TTGCCA |
| 3514E3-R14 | GGG GAA TAA CAG GAA GAA C |
| 3514E3-R15 | GCT GAA CCA TTG GCC |
| 3514E3-R16 | ATG TTG CAA AGC GCC |
| 3514E3-R17 | CGA TTA TTT CTA TTT ATG ACT CTT |
| | CTA TAA AGA TC |
| 3514E3-R18 | GCA TTA AGA AGA AGC AAC TTT CT |
| 3514E3-R19 | ATT CTT TTT TCG TTT CTC ACA ATA |
| | ATC |
| 3514E3-R20 | GTC AAA AAG AGA GTC TAC TGA TTC |
| 3514E3-R21 | GAA CCT TTG ACA ACC TTT CTT TTA |
| 3514E3-R22 | CGA CTT GAG AGG CCT |
| 3514E3-R23 | CCT GCT TAC CTT TTA ATG CAT |
| 3514E3-R24 | TTT TAC CAA GAA GAT TTT GCC TAA |
| 3514E3-R25 | CAA TAA CAG AAC GAC CAG AAT AA |
| 3517E3-F1 | TCT GCT TCT CAA AAT GTA AGAACA |
| 3517E3-F2 | TAA CCA AAT GCA CAT GTT ATCAAA |
| 3517E3-F3 | TTG CTG ATC AAG CTC AATAT |
| 3517E3-F4 | GCA ACT TAC AGA CGA AAT TAAT |
| 3517E3-F5 | AGA CAG AGG TTC TAT ACA AATTGA |
| 3517E3-F6 | AGG TAA CGG CAC ATA TTCAGA |
| 3517E3-F7 | TAA GAA TGA AGG AAT TGG CAGTT |
| 3517E3-F8 | AAT TTA AAT GAA GTA GAA AAA GTC |
| | TTAGT |
| 3517E3-F9 | GGC TAT TAA TTT TAT TCA GAC AACAGA |
| 3517E3-F10 | TTG TCA CAA GCT TCT AGA AATA |
| 3517E3-F11 | TTT CTG GTA AGA TTA ATG CTC AAAT |
| 3517E3-F12 | GAG CTT CTG ATG ATG CTGCT |
| 3517E3-F13 | GAA AAG CTT TCT AGT GGG TAC |
| 3517E3-F14 | CAT TAA CGC TGC TAA TCT TAG TAA |
| 3517E3-F15 | CAT CAG CTA TTA ATG CTT CAA GA |
| 3517E3-F16 | CAT GGA GGA ATG ATA TAT GAT TAT CAT |
| | G |
| 3517E3-F17 | TTT TTT TTT AAT TTT TGT GCT ATT CTT |
| | TTT AAC |
| 3517E3-F18 | TAA TAA TAA TTA TTT TTA ATG CTA TTG |
| | CTA TTT GC |
| 3517E3-F19 | ATT AAA GGC TTT TGA TTT TAA TCA AAG |
| | A |
| 3517E3-F20 | TTA AGC GCA TGA AAG ATC AAG |
| 3517E3-F21 | GTG GAA GGT GAA CTT AAT ACC |
| 3517E3-F22 | GAT TAT AAA AAG AAG TAC GAA GAT AGA |
| | GAG |
| 3517E3-F23 | TTA TTT TTT TGA TTA AAA ATT TTC AAG |
| | TCG TAA |
| 3517E3-F24 | GCT TCC GGA GGA GTT ATT TAT |
| 3517E3-F25 | TAG GAG ATT GTC TGT CGC |
| 3517E3-R1 | GCA ACA TTA GCT GCA TAA ATAT |
| 3517E3-R2 | TCC CTC ACC AGA GAAAAG |
| 3517E3-R3 | ACA CCC TCT TGA ACC GGTG |
| 3517E3-R4 | TGA GAA GGT GCT GTA GCAGG |
| 3517E3-R5 | TTG TAA CAT TAA CAG GAG AAT TAACTC |
| 3517E3-R6 | TTA GCA AGT GAT GTA TTA GCATCA |
| 3517E3-R7 | TGA TCA CTT ATC ATT CTA ATA GCATTT |
| 3517E3-R8 | CTA TTT TGG AAA GCA CCT AAAT |
| 3517E3-R9 | GCA TAC TCA GTA CTA TTC TTT ATAGAT |
| 3517E3-R10 | TGA GCA TAA GAT GCT TTT AGATTT |
| 3517E3-R11 | TCT GTC ATT GTA GCA TCT TTTA |
| 3517E3-R12 | TTA AAA TAC TAT TAG TTG TTG CTG CTAC |
| 3517E3-R13 | ATT AGC CTG CGC AATCAT |
| 3517E3-R14 | GCA ATG ACA AAA CAT ATT GGG |
| 3517E3-R15 | TTA ATA CAA TTT ATA CCA ATT AAA CTA |
| | GAA TTT T |
| 3517E3-R16 | ATA AAA AAA CAA AAG ATC CTT TAA AGG |
| | ATC |
| 3517E3-R17 | ATAAATTATACTAAAATTATTAAATTTTTGCCGAT |
| 3517E3-R18 | GCC TGC ATT ATG CTT TAT AAC A |
| 3517E3-R19 | CCT ACT CAA AGC AAA CTC C |
| 3517E3-R20 | CGA AAA TAC TTT ATA ACA ATC TTT AAT |
| | TTT AAC A |
| 3517E3-R21 | TCG ACT TAT CTG CTT TTT GTT AAC |
| 3517E3-R22 | CTA TCT TTG CCA TCT TCA TAG TC |
| 3517E3-R23 | GCA ATA AAA ATA GAA GAT TCT TTG TAG |
| | AT |
| 3517E3-R24 | TAA AAT TTC ATT TTC ATA AAC ATC AAG |
| | ATT AAT A |
| 3517E3-R25 | GCC CGA CAT ACC CA |
| 3511E3-F1 | CGTGAAGCTGCAAGAAAA |
| 3511E3-F2 | TGGAAAAGCAATAAAAGCTGCTG |
| 3511E3-F3 | TGTTGTATATGAACATTTATTGGAAAT |
| 3511E3-F4 | GCTTGGTAATTCTGAGATAAGAAA |
| 3511E3-F5 | CCTCAATTTGAAGGTCAAACAAA |
| 3511E3-F6 | ATTTTAAAGAGGGGCTTACAGCT |
| 3511E3-F7 | GCCATGAATGAAGCTTTTAAA |
| 3511E3-F8 | CTCATGTTATGGGATTTAGAAGTGG |
| 3511E3-F9 | CTGACAACATTCTTTCTTTTGTTAA |
| 3511E3-F10 | TGTTAATGTGGGGCTTAAATG |
| 3511E3-F11 | GCTTTTCAATCAGAACCTTATT |
| 3511E3-F12 | GAGGGTGGGATAAAATCTTTTT |
| 3511E3-F13 | TGGTAAAGAAAAATCTTCAAAATTTTAT |
| 3511E3-F14 | CGATAAAATATACATTTCAATTGAAGATAA |
| 3511E3-F15 | GGCTTAAAGAGCTTGCTTTT |
| 3511E3-F16 | AGATTATAATTTCGATGTTCTTGAAAAA |
| 3511E3-F17 | CGGATTCTGAAATTTTTGAAACTTT |
| 3511E3-F18 | GGGGACTAAGGTTACTTTTTT |
| 3511E3-F19 | AGAAGTTGTGGGGGAATCTTCTGTT |
| 3511E3-F20 | CTTTTTCAAAAGGTATTCCGACTT |
| 3511E3-F21 | GGTTTATGTTAATAGAGATGGAAAAAT |
| 3511E3-F22 | GGTTGTAAATGCTCTATCTTCGTT |
| 3511E3-F23 | TGGTAAGTTTAATAAAGGCACGTAT |
| 3511E3-F24 | CCT TGA ACT TGT TTT AAC AAA ATT AC |
| 3511E3-F25 | ACC GAT ATT CAT GAA GAG GAG |
| 3511E3-R1 | TACCCATTTTAGCACTTCCTCCA |
| 3511E3-R2 | TGGCAAAATGGCCTGAAAAA |
| 3511E3-R3 | TTGTTTTCTCAACATTAAGCATTTT |
| 3511E3-R4 | ATCATTGGTGATAACCTTATCTTCT |
| 3511E3-R5 | ACTCCTGCACCAAGAGAT |
| 3511E3-R6 | ATCTTGTGATAACGAAGTTTTGTA |
| 3511E3-R7 | TCCATCAACATCGGCATCTG |
| 3511E3-R8 | AAAAGCTAAAAGCAAAGTTCTAAT |
| 3511E3-R9 | ATATATCCATTTTCAATTAAATCTCTCAT |
| 3511E3-R10 | TATAAAGAGGAGGCATGGCT |
| 3511E3-R11 | TAAAAATAATAAATACGATTGTCATACTTT |
| 3511E3-R12 | TATTGCGATTTTTAGTTTCAATAGAA |
| 3511E3-R13 | CCCAAGCCCTTTATATCTCTGAA |
| 3511E3-R14 | AGCTGCGTTGGATTCATC |
| 3511E3-R15 | CTAGCAGGATCCATAGTTGTTT |
| 3511E3-R16 | ATCATCTATATTCATCAATCTCATTTTT |
| 3511E3-R17 | GAGTAACAAAAATTTTTTCAGCTTCA |
| 3511E3-R18 | TTTCTGGGCTCAACTAAATCT |
| 3511E3-R19 | GATTAATTACATTAAGTGCATTCTGTTC |
| 3511E3-R20 | CCATTAACGCTCCAATTACAC |
| 3511E3-R21 | TCCTAACATTTAATATTTGTTCTTTATTTTC |
| 3511E3-R22 | GCATAATTTAAATAAGAAGTTTTTATTTCATCT |
| 3511E3-R23 | GAAGAGCTCTAGAAACAATAACTGA |
| 3511E3-R24 | TGGTTTAAGACCATCTCTTACGT |
| 3511E3-R25 | CTC ATA CAT AGA ATA AAG TAT TCT CCT |
| | G |

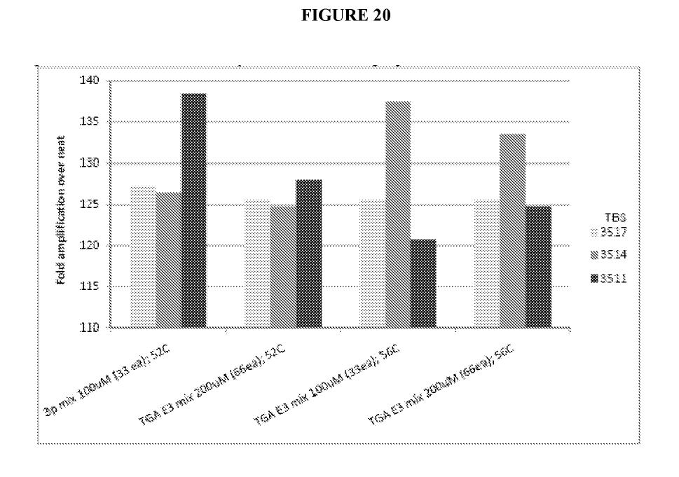

Human DNA was extracted from 1 ml blood to result in 200 μA DNA extract. The equivalent of 50 copies of B. burgdorferi B31 genome was added to each reaction. Amplification reactions were set up, each with a total volume of 225 μl as described supra. Primer extension was conducted for 4 hours at the annealing temperatures indicated in FIG. 20, followed by incubation for 20 minutes at 80° C. and hold at 4° C. Five microliters of each sample were analyzed with using a TBS 5.0 plate. Results are shown in FIG. 20, where “3p mix” refers to “set E” primers.

An expanded set of primers was developed to encompass 7 different regions for detection of Borrelia target DNA, as shown in Table 27.

TABLE 27
Additional primers used for TGA amplification of B. burgdorferi
target regions 3519 (SEQ ID NOS 2362-2411, respectively, in
order of appearance), 3520 (SEQ ID NOS 2362-2411, respectively,
in order of appearance), 3516 (SEQ ID NOS 2412-2461, respectively,
in order of appearance), 3515 (SEQ ID NOS 2462-2511, respectively,
in order of appearance), and 3518 (SEQ ID NOS 2512-2561,
respectively, in order of appearance). These primers referred
to as the “8E3 set” in combination with the previous “E3 set”
(Table 26).

| Primer name | Sequence |
|---|---|
| 3519-20E3-F1 | CCC ACA CTC TCT CTT TCA AA |
| 3519-20E3-F2 | GAT ATT AAC CGG CAT TTA ACC TT |
| 3519-20E3-F3 | TCT AGC TTA CAA TCC CAT TTA TAA GA |
| 3519-20E3-F4 | CCT TCA AAT TTT AAT TTT CCT CTA AAA GTT A |
| 3519-20E3-F5 | CCT TCA AAA GAA GAA TCA AGA TAC AA |
| 3519-20E3-F6 | CAC ACC CCC TTT TGA AGA TA |
| 3519-20E3-F7 | GTA ATA ACC TTA CTA TTC TTG CCA ATA |
| 3519-20E3-F8 | TTC TAC TAT TAA TGT ATC ACA AAT TAC CAC |
| 3519-20E3-F9 | GCA TTT ACA TTG CCC TTC AA |
| 3519-20E3-F10 | CAA CCG CTG TTT AAA TAA ACC TT |
| 3519-20E3-F11 | AAT ATT TTT TTT GTT TTT ACA TCC CCA TAT |
| 3519-20E3-F12 | CAC ACT TAC CAT CAA AAA TTA TAT TAT CAT |
| 3519-20E3-F13 | AAG AAA ATA AAT CTA CAA TTT CAT TAG ACT TTA |
| 3519-20E3-F14 | CAA AGT ATC TTT TAT TTG TGA AAC GG |
| 3519-20E3-F15 | TCT ACT TAT TAT TAA TTA ATA AAA AAC ACT GAC C |
| 3519-20E3-F16 | CTC TAC GAA TTA AAT TTT TAA GAA AGG ATT TTA |
| 3519-20E3-F17 | ATC AAA TCC ACC ATT TTT TTT ATC CA |
| 3519-20E3-F18 | CCA ACC GCC TTA TTT CAC |
| 3519-20E3-F19 | TTT TCA AAT TAT CTT CAA TCT TAA ACT CTT TAG |
| 3519-20E3-F20 | TTT TAG CAA CAA CTT TAA CCA CTT T |
| 3519-20E3-F21 | TGT CAC GCT AGA TGC AG |
| 3519-20E3-F22 | CTT TAC GCC ACT TAA ATC TGC |
| 3519-20E3-F23 | AAT CAG AAA ATA TTA CCC CGT TTG |
| 3519-20E3-F24 | ATA TTA TTT TCT AAA CCT GAA GAA GGA ATA T |
| 3519-20E3-F25 | CAT TAA AAA ATT TGA TGA TAT TAC TTT GCT C |
| 3519-20E3-R1 | GTT TTG CTG TTA AAG TAA GGA AAT TAG |
| 3519-20E3-R2 | GCT GCT AGA AAA AAA TCT CGT T |
| 3519-20E3-R3 | CTG CTA GAA AGC GAA TAA TTC ATA A |
| 3519-20E3-R4 | GAA TTT TTT AAA TTT GTT GCA AAA AAA CTA G |
| 3519-20E3-R5 | GCG GGT AAG AAA GAC GAA |
| 3519-20E3-R6 | GAA AAA CGC TGT ATC AAC ATG A |
| 3519-20E3-R7 | ATT AGA AAT GTA AGT GTA AAA AGT GAA TTA AAA |
| 3519-20E3-R8 | CGC TCT CGT CAA AAT TTA AAA AG |
| 3519-20E3-R9 | CTT GAG AAA AAA TGC ATC TGC |
| 3519-20E3-R10 | GAT ATA TTA AAG CTA TTG TTT AAT AAT ATT ATT AAG GA |
| 3519-20E3-R11 | ATT AAC TTA AAT CTT TGA TTG ACT ATA TTT GAA T |
| 3519-20E3-R12 | AGG TTT TTG AAT ATA TTA ATC AAA ACT ATT GT |
| 3519-20E3-R13 | ATT TTG AAT AAA AAA ATT TCT TAT TCC ATG C |
| 3519-20E3-R14 | CAA AGA AAA TCA TCA GAC AAA AAA GG |
| 3519-20E3-R15 | GAA TTT GAA TTT AAC AAT AAA AAT TAT TTA TGC TT |
| 3519-20E3-R16 | GAA TTT TTT GAA AAA ATT TTT ATT GCC AG |
| 3519-20E3-R17 | CTG TGA AAG AAA AAT TTT TAA AAG TGA AAT |
| 3519-20E3-R18 | CAA TAT AGT GTT ATT TTA TGA GTT TAG GAA AG |
| 3519-20E3-R19 | TTT TGT TGG GGG ATT TTT CAG |
| 3519-20E3-R20 | TTT TGT TGG GGG ATT TTT CAG |
| 3519-20E3-R21 | GCA ATA TAT TTA TTT TTT TAT TTA TTT GTT TTA TTG ATA |
| | TTA |
| 3519-20E3-R22 | GTA TTA TGA TTG CTT TAT TTG TTT ATT ACA TTT C |
| 3519-20E3-R23 | GAT ATT ATT TAT CTT GTA CTT ATC TTT TTA TGT TTT |
| 3519-20E3-R24 | GTC CCA AAA TTG GAA AAT TTT CC |
| 3519-20E3-R25 | TAT TTA AAG AGC TTA AAA TTA AGA GAA AAG ATC |
| 3516E3-F1 | CCT ATC CTT CTG CCA ACG |
| 3516E3-F2 | GGA AAA AAG ATT GTA TAT ACT TGA CAT G |
| 3516E3-F3 | CAA AGT AGA AGA AGA TCC AAG TAT TC |
| 3516E3-F4 | GGC AAG AAT TTT GGG ATA ATA ACA |
| 3516E3-F5 | TGT CTA AAT ACG AAT TCA TAA AAA TTG AAA |
| 3516E3-F6 | GAA TAA GTT GTT ATG AGT TAA TTT TCA AGA |
| 3516E3-F7 | TAA ATA AAA TTA ACA AAA ATG CAA TCT AAA AGA A |
| 3516E3-F8 | CAC AGC ATC AAA ATT GTT AGC |
| 3516E3-F9 | CCG TGC TGG TTC AAG |
| 3516E3-F10 | GAG GGG CTA GTG GG |
| 3516E3-F11 | GGA AGT GGT AGA CAC GC |
| 3516E3-F12 | AAA AAA TAT AAT GGT TAA TAG TGC TGT G |
| 3516E3-F13 | GTA ATT AAA AAA AAT AAA AAA GTT GAC AAA AAT T |
| 3516E3-F14 | CGC TGT AAT AGC AAC AAC AAT AAT A |
| 3516E3-F15 | AAT AAT ATT TTC AAA AAT AAA AAT AAT TAT ATT TGC AA |
| 3516E3-F16 | CTC TAA GCT TCA AAC TAG GTC A |
| 3516E3-F17 | AAC TTT GCT CTC AAT AGT TGT TT |
| 3516E3-F18 | TAT GAA ATC TAA TCT ATT TAT TGT TTC TGA CT |
| 3516E3-F19 | GCA ATA TTT ATG TCA GCA GGA A |
| 3516E3-F20 | GAA GGT TTA TAC CCT TTG GAG |
| 3516E3-F21 | TGG TCA ATA TGG GGT ATT AAC TTT AT |
| 3516E3-F22 | CAA TAA CCT GCT TGA CAA AAT AAA TTA |
| 3516E3-F23 | GGA AAT TAA TGG GAA ATA AAT TAT TTA AAA ACA |
| 3516E3-F24 | AGA CAT AAT ATC TTT TTA CAT TGG GAA A |
| 3516E3-F25 | TTT AGG AAT TTT TTG GGG AGC |
| 3516E3-R1 | TGA GGG TGA GTT CCT GT |
| 3516E3-R2 | TTG TTT TTT AAA CTT ATT AAT ATT TTC TTC TGT AC |
| 3516E3-R3 | GGC AAA CCC CAA AGC |
| 3516E3-R4 | GTG TTC TAA TTT CTC GAT CCC |
| 3516E3-R5 | ACT GTG TCC ATT TAT AGT AAT TCT C |
| 3516E3-R6 | CTA GCC CTT TTT TAT ATA ATT GCA GG |
| 3516E3-R7 | GTA CCA TAC AGG CAT TTC TTT TA |
| 3516E3-R8 | CTA GTA CCG TTC CAA GCT |
| 3516E3-R9 | GTC CAT CTG AAG TTT GAA TAA TCT C |
| 3516E3-R10 | ACG CTG TAA GAT CCT CTC |
| 3516E3-R11 | GTA ATT TTT AAA ACC CAC TGT CTT AAA TA |
| 3516E3-R12 | CGT CTA GCA ATC TTT CAG C |
| 3516E3-R13 | AGA TTC AGG CCA TTC TAA TTC T |
| 3516E3-R14 | TCC AAT TTC GCT GCA TTT C |
| 3516E3-R15 | AAT TCA ATT TCA ACT CCT GTT GAT |
| 3516E3-R16 | TTT TAT CGC TGT GGC CTT |
| 3516E3-R17 | CTT TAC AAC AAG GCC AGA C |
| 3516E3-R18 | CGA TCA CTA AAT ATG TGA TGC C |
| 3516E3-R19 | GTT GTT CTT TGT TAT TTT TTC TAT TAG TTT ATT |
| 3516E3-R20 | CTT CGT GCT TTA CAT ATT TTA AAA CAT T |
| 3516E3-R21 | GAG AAG TCC TAT TAA GAT CGC T |
| 3516E3-R22 | CTG TGA AAA CTC CCG ATT TAT C |
| 3516E3-R23 | CAT TTG TTA TTG GAT GAA ATG CG |
| 3516E3-R24 | GCT TCC AAC CCA AAT TG |
| 3516E3-R25 | CGG TTC CGT AAG TTC CT |
| 3515E3-F1 | GTG TTG CTA TGA ATC CTG TTG |
| 3515E3-F2 | GGT AGA AGA CCC AAG GT |
| 3515E3-F3 | GGA AAG CTG GTA AAA GTA GG |
| 3515E3-F4 | TGG AAA TGA AGA TTA TGC CAA TAT TT |
| 3515E3-F5 | TTT TTA AAA AAT GTA TTG CAA CAA TTG G |
| 3515E3-F6 | TTA TCA TCT GGC GAG ATG AG |
| 3515E3-F7 | GAC GGG AAT TAT GTC ACT G |
| 3515E3-F8 | GGT GGA TAT GCT ATG ATA CTT G |
| 3515E3-F9 | GGG TGG ACA GCT TAT AAG A |
| 3515E3-F10 | CGT TCA CAA TAT TGA GCT TAA TGT |
| 3515E3-F11 | CTT ACC TCT TGA AAA TAT TCC TAT TGG |
| 3515E3-F12 | CTA ATG CTC CAA TTA AAA TTG GC |
| 3515E3-F13 | GGT TGG AGA TGT TTT GGA AAG |
| 3515E3-F14 | AAA GGT ATA TTA TTT CTC CTA AAG GC |
| 3515E3-F15 | GCT AAT ATA GCT TTG CTT GTT TAT AAA G |
| 3515E3-F16 | GTT GCT TCT ATT GAA TAT GAT CCT AA |
| 3515E3-F17 | CGA AGA GAT AAA TTT AGC ATT CCT G |
| 3515E3-F18 | GCA TAA GAG AAA GTA TAG GTT GAT TG |
| 3515E3-F19 | CTG GTA GGA TTA GTA TTA GAA GAA GAG |
| 3515E3-F20 | AAA AGG TAA AAA ATT TAA ATC GGG C |
| 3515E3-F21 | CAA AGG TAA TGA TCC TTT GAA ATC |
| 3515E3-F22 | GCT ATA AGA CGA CTT TAT CTT TTG AT |
| 3515E3-F23 | AGA CTT ATA AGC CAA AAA CTT CTT C |
| 3515E3-F24 | GGT TTT GGA GAA AAA TAA ATA TGG G |
| 3515E3-F25 | CAA AAA GGA AGA TAA AAT AGA TAT TTT TTA GTG |
| 3515E3-R1 | TTT CTT GCG AGT CTT ATA ACC T |
| 3515E3-R2 | TTT ATT TCT TCT TTT AAT AAT AAA TTT ATC TGA ATA TC |
| 3515E3-R3 | TTT TTT AAT AGA TCT TGC CAC TAT ACT C |
| 3515E3-R4 | ACT TTT TGA TAA AGA CTC TTT TCT ATA AAA G |
| 3515E3-R5 | CTT CTC ACT TCC AAA AGA CG |
| 3515E3-R6 | AAG ATC TGG AGT AGG TTT TAA TAA C |
| 3515E3-R7 | GGC TTA CCA TTT CAG GAA TTA T |
| 3515E3-R8 | AAG TTT TGC CAT TGT AAA CAG ATA T |
| 3515E3-R9 | CAA GAT CCT CGG TAA TAT AAA TAG GT |
| 3515E3-R10 | CTC GCC AAG CTT ATG TC |
| 3515E3-R11 | CCT CTA AAA ATC CTT GTA GGT G |
| 3515E3-R12 | CCT CTA AAA ATC CTT GTA GGT G |
| 3515E3-R13 | CTT CCC TTT TTA TCT GAC TTA GC |
| 3515E3-R14 | ATC TTC TAT TTA CCA ACA TAA CTA CTT AC |
| 3515E3-R15 | AGG GTA AAT TTT TGC CCT TTG |
| 3515E3-R16 | CTA TTG GCC TAA CTT TTT TTG G |
| 3515E3-R17 | AGA CTC TCC CCG GAT ATT |
| 3515E3-R18 | AAA GCA CTG CAA TAG CC |
| 3515E3-R19 | TAA AAG CTT AGC TCC TTT ATT AGG |
| 3515E3-R20 | TGA TGC TGC TGA CTT AAC AA |
| 3515E3-R21 | CTC GGA AAG ATT TTT ATT GTG ATA CA |
| 3515E3-R22 | CAT CAA CCA TAA CTG TTT TAA CAA ATA TC |
| 3515E3-R23 | GCC AAA TCT TTT TAC GAC GAC |
| 3515E3-R24 | TAT CAG CTC TAC CCC TAG C |
| 3515E3-R25 | CTT CAA CAA AAA TAT GAC AAT TTC TAT TAA C |
| 3518E3-F1 | TTA ATG AAA AAG AAT ACA TTA AGT GCA ATA T |
| 3518E3-F2 | CTA ATA ATT CAT AAA TAA AAA GGA GGC AC |
| 3518E3-F3 | TTT TCA AAT AAA AAA TTG AAA AAC AAA ATT GT |
| 3518E3-F4 | AAT ATT TAT TCA AGA TAT TGA AGA ATT TGA AAA A |
| 3518E3-F5 | TTT AAA ATC AAA TTA AGA CAA TAT TTT TCA AAT TC |
| 3518E3-F6 | AGC ATA TTT GGC TTT GCT TAT G |
| 3518E3-F7 | AAA TTA AAA CTT TTT TTA TTA AAG TAT ACT TCA TTT AA |
| 3518E3-F8 | GCC TGA GTA TTC ATT ATA TAA GTC C |
| 3518E3-F9 | TAT ATT GGG ATC CAA AAT CTA ATA CAA G |
| 3518E3-F10 | CAA TTT CTC TAA TTC TTC TTG CAA TTA G |
| 3518E3-F11 | GGA GTA TAG TAA GGT ATT ACT TTT GTA TAA A |
| 3518E3-F12 | TTC CTG AGA TAT TCA TAT TTT TAA TTT CTT TT |
| 3518E3-F13 | GCA GGA CTT CCA CTT AGT A |
| 3518E3-F14 | GGT AGG AGC TTC TTT TGA ATA AAC |
| 3518E3-F15 | CAA AAT AGG TAT TTT CAA ATT AAA AAT TTC CAT A |
| 3518E3-F16 | AAT TTA ACA ATT ATT TGC ATT CCA TAA CAT A |
| 3518E3-F17 | GCT TAG AGT CTT TAG ATA CTA GGC |
| 3518E3-F18 | AAA GAT TTC AGA GCT CCC ATA T |
| 3518E3-F19 | TTC TGA AAA TAA AAG AGA TTT TTC ATC TC |
| 3518E3-F20 | TGA CTC ATG ATA ATT TGA AAT TTG TTT G |
| 3518E3-F21 | AAA TTA TCA GGA ATT TTT TCA ATA CTG TC |
| 3518E3-F22 | GCA ATA CAA TTT TTT GTA AAA GCT AAT TG |
| 3518E3-F23 | CCG TAA ATT TTT TGA GTT TCA TTT GAT |
| 3518E3-F24 | AGT TAC TTC TGG ATG GAA TTG T |
| 3518E3-F25 | AAT TTT TAA TTA TTT GAT CAC CAA ATT CAG |
| 3518E3-R1 | ACC GCA TTA GAA TCC GTA AT |
| 3518E3-R2 | ACC TCT TTC ACA GCA AGT T |
| 3518E3-R3 | CAT CTA TAG ATG ACA GCA ACG |
| 3518E3-R4 | TTA CCA ATA GCT TTA GCA GCA |
| 3518E3-R5 | GTA TCC AAA CCA TTA TTT TGG TGT A |
| 3518E3-R6 | GCT AAC AAT GAT CCA TTG TGA TTA T |
| 3518E3-R7 | TAT TAG GGT TGA TAT TGC ATA AGC |
| 3518E3-R8 | CTT CAT TTT TCA ATC CAT CTA ATT TTT G |
| 3518E3-R9 | TTA GCC GCA TCA ATT TTT TCC |
| 3518E3-R10 | TCT TTT AAT TTA TTA GTA AAT GTT TCA GAA CA |
| 3518E3-R11 | CTT CTT TAC CAA GAT CTG TGT G |
| 3518E3-R12 | CTT TTG CAT CAG CAT CAG T |
| 3518E3-R13 | TTT AGT TTT AGT ACC ATT TGT TTT TAA AAT G |
| 3518E3-R14 | GAT TCA AAT AAT TTT CCA AGT TCT TCA G |
| 3518E3-R15 | CTG CTT TTG ACA AGA CCT C |
| 3518E3-R16 | TTT AAC TGA ATT AGC AAG CAT CTC |
| 3518E3-R17 | CCA CAA CAG GGC TTG |
| 3518E3-R18 | GAT CTT AAT TAA GGT TTT TTT GGA CTT |
| 3518E3-R19 | CCA GTT ACT TTT TTA AAA CAA ATT AAT CTT ATA |
| 3518E3-R20 | AGA AAT CTT TCT TGA CTT ATA TTG ACT TT |
| 3518E3-R21 | GAA TTT TAA GAA ATT TTT TGA GAA AAT AAA AAA ATA AAA |
| 3518E3-R22 | TAT TCT TTA AGA GAA GAG CTT AAA GTT |
| 3518E3-R23 | AAA TTC AAT TTA TTA ACG GCT TTT GTA ATA |
| 3518E3-R24 | TCT AGC ACC CAA TTT TGT TTA TAT TTA |
| 3518E3-R25 | GTT TAA GCC TAC TTA AAG TCT TTA AAA TC |

The new 8E3 primer set was tested for its ability to amplify Borrelia burgdorferi DNA and compared to the previously used E3 primer set, which targeted 3511, 3514, and 3517. Amplification samples were set up as described previously, where 30 genome copies B. burgdorferi were added to 200 μl human DNA extracted from 1 ml blood, and total reaction volume was 225 μl. Primer sets were added as shown in Table 28. Samples were mixed (with the exception of polymerase) in 0.6 ml PCR tubes, denatured at 95° C. for 10 minutes, cooled to 60° C., and then Bst polymerase was added. Samples were mixed by vortexing, centrifuged briefly, then incubated at 56° C. for 4 hours. Polymerase was inactivated by incubation at 80° C. for 20 minutes, and samples were held at 4° C. until analysis.

TABLE 28
Primer sets and primer concentrations
used for experiment shown in FIG. 10.

| | Primer | Initial primer | Final |
|---|---|---|---|
| Mix | Set | conc (uM) | conc (uM) |
| 1 | E3 | 1000 | 10 |
| 2 | E3 | 100 | 1 |
| 3 | 8E3 | 1000 | 30 |
| 4 | 8E3 | 1000 | 10 |
| 5 | 8E3 | 100 | 1 |
| 6 | 8E3 | 100 | 0.2 |

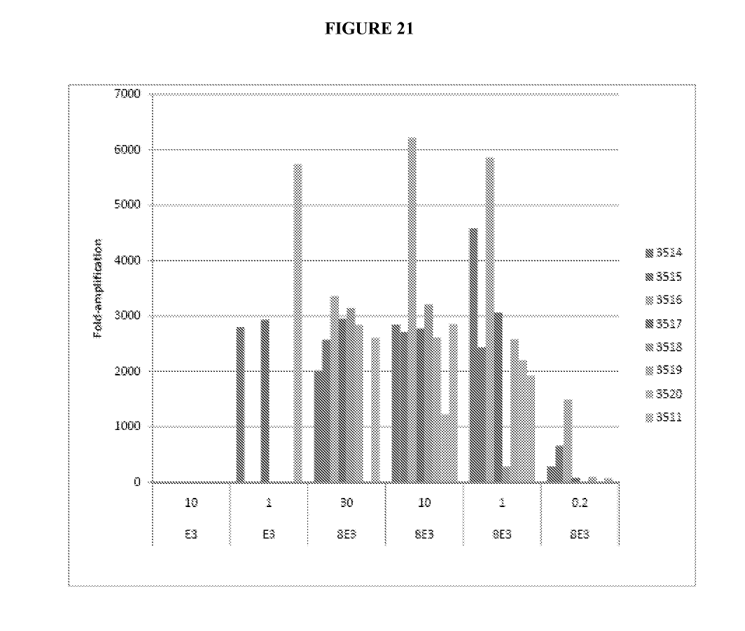

Following TGA amplification, samples were treated with calf intestinal phosphatase (CIP) by adding 2 μA CIP per reaction, followed by vortexing, brief centrifugation, and incubation at 37° C. for 30 minutes, then heat inactivation at 95° C. for 15 minutes and hold at 4° C. Samples were then analyzed using a Borrelia Multilocus Sequence Typing (MLST) genotyping plate using 5 μl of each sample per well, followed by processing on an Eppendorf procycler and analysis on a PlexID unit. Results are shown in FIG. 21. The 8E3 primer set provided a similar level of amplification to the E3 primer set for the different MLST targets. There was little variation in fold amplification noted when the final concentration of primer mix was varied from 30 μM to 1 μM.

The methods were then applied on a series of clinical blood samples from human patients with suspected Lyme Borreliosis. Two sets of DNA extractions were performed. The first set of extractions was analyzed directly on the Borrelia MLST plate described herein, while the other set of extractions was first amplified using the TGA method and then subjected to analysis with the Borrelia MLST plate. The TGA reactions were set up using 1× PCR buffer, 199.3 μl sample, 0.2 mM (each) dNTPs, primers as indicated infra, and 0.05 U/μl Bst polymerase. Results are shown in Table 29. In total, signal was observed for 2 of 29 unamplified samples, but 14 of 29 amplified samples. The TGA treatment increased the sensitivity of detection of Lyme borreliosis in blood. 12 samples that were negative when run untreated were found to be positive for Borrelia burgdorferi after TGA treatment.

TABLE 29
Analysis of human clinical (blood) samples
for Borrelia MLST signals.

| | Neat untreated | Borrelia TGA |
|---|---|---|
| | (Detected/Total | (Detected/Total |
| Sample | Primers) | Primers) |
| JHU 01-020.v1 | 0/8 | 0/8 |
| JHU 01-023.v1 | 0/8 | 0/8 |
| JHU 01-024.v1 | 0/8 | 0/8 |
| JHU 01-025.v1 | 0/8 | 0/8 |
| JHU 01-026.v1 | 0/8 | 8/8 |
| JHU 01-027.v1 | 0/8 | 0/8 |
| JHU 01-028.v1 | 0/8 | 0/8 |
| JHU 01-029.v1 | 0/8 | 0/8 |
| JHU 01-030.v1 | 0/8 | 0/8 |
| JHU 01-031.v1 | 0/8 | 0/8 |
| JHU 01-032.v1 | 0/8 | 0/8 |
| JHU 01-033.v1 | 0/8 | 8/8 |
| JHU 01-034.v1 | 0/8 | 0/8 |
| JHU 01-036.v1 | 0/8 | 1/8 |
| JHU 01-037.v1 | 0/8 | 8/8 |
| JHU 01-039.v1 | 0/8 | 0/8 |
| JHU 01-040.v1 | 0/8 | 7/8 |
| JHU 01-042.v1 | 0/8 | 0/8 |
| JHU 01-044.v1 | 0/8 | 6/8 |
| JHU 01-045.v1 | 0/8 | 6/8 |
| JHU 01-046.v1 | 0/8 | 8/8 |
| JHU 01-047.v1 | 0/8 | 8/8 |
| JHU 01-048.v1 | 0/8 | 0/8 |
| JHU 01-049.v1 | 0/8 | 1/8 |
| JHU 01-050.v1 | 0/8 | 0/8 |
| JHU 01-051.v1 | 1/8 | 8/8 |
| JHU 01-052.v1 | 0/8 | 8/8 |
| JHU 01-053.v1 | 3/8 | 8/8 |
| JHU 01-054.v1 | 0/8 | 3/8 |
| JHU 02-J-1 (negative | 0/8 | 0/8 |
| control blood) | | |
| JHU 02-S-1 (negative | 0/8 | 0/8 |
| control blood) | | |
| JHU 02-506 (negative | 0/8 | 0/8 |
| control blood) | | |
| Samples positive for | 2 of 29 | 14 of 29 |
| B. burgdorferi | | |

Samples were unamplified (“neat”) or subjected to amplification (“TGA”).

### CONCLUDING STATEMENTS

The present invention includes any combination of the various species and subgeneric groupings falling within the generic disclosure. This invention therefore includes the generic description of the invention with a proviso or negative limitation removing any subject matter from the genus, regardless of whether or not the excised material is specifically recited herein.

While in accordance with the patent statutes, description of the various embodiments and examples have been provided, the scope of the invention is not to be limited thereto or thereby. Modifications and alterations of the present invention will be apparent to those skilled in the art without departing from the scope and spirit of the present invention.
The contents of each reference (including, but not limited to, journal articles, U.S. and non-U.S. patents, patent application publications, international patent application publications, gene bank gi or accession numbers, internet web sites, and the like) cited in the present application are incorporated herein by reference in their entirety.

We claim:

1. A method of amplifying a target sequence comprising:
a) contacting a sample with a strand displacing polymerase, a first upstream primer, a second upstream primer, a first downstream primer, and a second downstream primer, wherein said sample is suspected of containing a nucleic acid sequence comprising a target region sequence, wherein said first and second upstream primers are able to hybridize to said nucleic acid sequence upstream of said target region sequence, and wherein said first and second downstream primers are able to hybridize to said nucleic acid sequence downstream of said target region sequence; and
b) treating same sample under conditions such that:
i) a first upstream amplicon is generated comprising said first upstream primer and said target region sequence,
ii) a second upstream amplicon is generated that comprises said second upstream primer, the sequence of said first upstream primer, and said target region sequence, wherein said first upstream amplicon is strand displaced by said strand displacing enzyme during the generation of said second upstream amplicon;
iii) a first downstream amplicon is generated comprising said first downstream primer and said target region sequence, and
iv) a second downstream amplicon is generated that comprises said second downstream primer, the sequence of said first downstream primer, and said target region sequence, wherein said first downstream amplicon is strand displaced by said strand displacing enzyme during the generation of said second downstream amplicon.

2. The method of claim 1, wherein said method further comprises detecting the presence or absence of said first upstream amplicon, said second upstream amplicon, said first downstream amplicon, said second downstream amplicon, or any combination thereof.

3. The method of claim 1, wherein said treating is incubating said sample under isothermal conditions.

4. The method of claim 1, wherein said strand displacing polymerase is selected from the group consisting of Phi 29, Klenow polymerase, and Bst polymerase.

5. The method of claim 1, wherein said strand displacing polymerase is Bst polymerase.

6. The method of claim 1, wherein said sample is selected from the group consisting of a biological sample, an environmental sample, a synthetic sample, and a manufactured sample.

7. The method of claim 1, wherein said sample is a biological sample selected from the group consisting of blood, serum, plasma, tissue, cells, saliva, sputum, urine, cerebrospinal fluid, pleural fluid, milk, tears, stool, sweat, semen, whole cells, cell constituent, cell smear, and extracts thereof.

8. The method of claim 1, wherein said target region sequence is present in a spirochete genome.

9. The method of claim 8, wherein said spirochete is a member of the genus Borrelia.

10. The method of claim 1, wherein said sample is contacted with at least 5 upstream primers and 5 downstream primers.

11. The method of claim 1, wherein said sample is contacted with at least 10 upstream primers and 10 downstream primers.

12. The method of claim 1, wherein the average Tₘ of said primers is in the range of 35-60° C.

13. The method of claim 1, wherein said displaced strand of said first upstream amplicon functions as template for amplification by a downstream primer.

14. The method of claim 1, wherein said displaced strand of said first downstream amplicon functions as template for amplification by an upstream primer.

15. The method of claim 2, wherein said detecting is conducted using a method selected from the group consisting of a PCR method, a mass spectrometry method, and a sequencing method.

16. A kit for use in conducting the method of claim 1, said kit comprising two upstream primers, two downstream primers, and a strand-displacing polymerase.

17. A method of amplifying a target sequence comprising:
a) contacting a sample with a strand displacing polymerase, at least two upstream primers, and at least two downstream primers, wherein said sample is suspected of containing a nucleic acid sequence comprising a target region, wherein said at least two upstream primers hybridize to said nucleic acid sequence upstream of said target region, and wherein said at least two downstream primers hybridize to said nucleic acid sequence downstream of said target region; and
b) treating same sample under conditions such that amplicons are generated from said at least two upstream primer and from said at least two downstream primers.

18. The method of claim 17, wherein said at least two upstream primers comprises at least 5 upstream primers and wherein said at least two downstream primers comprise at least 5 downstream primers.

19. The method of claim 17, wherein said strand displacing polymerase is selected from the group consisting of Phi 29 base, Klenow polymerase, and Bst polymerase.
